SEQUENCE LISTING (83 sequences)

SEQ ID NO: 1 - protein, 7 aa, Artificial Sequence
  anti-ASPH antibody CDRH1
SNSAAWN

SEQ ID NO: 2 - protein, 18 aa, Artificial Sequence
  anti-ASPH antibody CDRH2
RTYYRSKWYNDYAVSVKS

SEQ ID NO: 3 - protein, 13 aa, Artificial Sequence
  anti-ASPH antibody CDRH3
TGYSSSWVVNFDY

SEQ ID NO: 4 - protein, 13 aa, Artificial Sequence
  anti-ASPH antibody CDRL1
SGSSSNIGSNYVY

SEQ ID NO: 5 - protein, 12 aa, Artificial Sequence
  anti-ASPH antibody CDRL2
KLLIYKNNQRPS

SEQ ID NO: 6 - protein, 11 aa, Artificial Sequence
  anti-ASPH antibody CDRL3
AAWDDSLRGYV

SEQ ID NO: 7 - protein, 7 aa, Artificial Sequence
  anti-ASPH antibody CDRH1
SDSAAWN

SEQ ID NO: 8 - protein, 12 aa, Artificial Sequence
  anti-ASPH antibody CDRH3
AQNNIAVAGFDY

SEQ ID NO: 9 - protein, 12 aa, Artificial Sequence
  anti-ASPH antibody CDRL2
TLLIYRNNQRPS

SEQ ID NO: 10 - protein, 12 aa, Artificial Sequence
  anti-ASPH antibody CDRL3
AAWDDSLSGLYV

SEQ ID NO: 11 - protein, 7 aa, Artificial Sequence
  anti-ASPH antibody CDRH1
ADRVAWN

SEQ ID NO: 12 - protein, 18 aa, Artificial Sequence
  anti-ASPH antibody CDRH2
RIFYRSKWMVDYAVSVKS

SEQ ID NO: 13 - protein, 9 aa, Artificial Sequence
  anti-ASPH antibody CDRH3
ATTRGYFDL

SEQ ID NO: 14 - protein, 18 aa, Artificial Sequence
  anti-ASPH antibody CDRH2
RTYYRSKWYNGYAVSVRG

SEQ ID NO: 15 - protein, 13 aa, Artificial Sequence
  anti-ASPH antibody CDRH3
TGYSSSWVVNSNY

SEQ ID NO: 16 - protein, 135 aa, Artificial Sequence
  anti-ASPH CAR antigen-binding fragment VH region
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARTGYSSSWVVNFDYWGQGTLVTVSSGSASAPTGIL

SEQ ID NO: 17 - protein, 112 aa, Artificial Sequence
  anti-ASPH CAR antigen-binding fragment VL region
QPVLTQSPSASGTPGQRVTISCSGSSSNIGSNYVYWYQQLPGTAPKLLIYKNNQRPSGVPDRFSGSKSGTAASLAISGLQSEDEADYYCAAWDDSLRGYVFGTGTKLTVLSG

SEQ ID NO: 18 - protein, 127 aa, Artificial Sequence
  anti-ASPH CAR antigen-binding fragment VH region
QVQLQQSGPGLVKPSQTLSLTCAISGDSVSSDSAAWNWIRQSPSRGLEWLGRTYYRSKWYNDYAVSVKSRISINPDTSKNQFSLQLNSVTPEDTAVYYCARAQNNIAVAGFDYWGLGTLVTVSSGIL

SEQ ID NO: 19 - protein, 115 aa, Artificial Sequence
  anti-ASPH CAR antigen-binding fragment VL region
QPVLTQSPSASGTPGQRVTISCSGSSSNIGSNYVYWYQQLPGTAPTLLIYRNNQRPSGVPDRFSGSKSGTSASLAISGLRSEDEAEYYCAAWDDSLSGLYVFGTGTKVTVLSGIL

SEQ ID NO: 20 - protein, 124 aa, Artificial Sequence
  anti-ASPH CAR antigen-binding fragment VH region
QVQLQQSGAGLVKPSQTLSLTCTISGDSVSADRVAWNWIRQSPLRGLEWLGRIFYRSKWMVDYAVSVKSRISINPDTSKNQFSLQLNSVTPEDTAMYYCARATTRGYFDLWGRGTLVTVSSGIL

SEQ ID NO: 21 - protein, 135 aa, Artificial Sequence
  anti-ASPH CAR antigen-binding fragment VH region
QVQLQQSGPGLVKPSPTLSLTCAISGDSVSSNSAAWNWVRQSLSRGLEWLGRTYYRSKWYNGYAVSVRGRITTNADTSRNQFSLQLNSVTPEDTAVYYCARTGYSSSWVVNSNYWGQGTLVTVSSGSASAPTGIL

SEQ ID NO: 22 - protein, 110 aa, Artificial Sequence
  anti-ASPH CAR antigen-binding fragment VL region
QPALTQSPSASGTPGQRVTISCSGSSSNIGSNYVYWYQQLPGTAPKLLIYKNNQRPSGVPGRFSGSKSGTAASLAISGLRSKDEADYYCAAWDDSLRGYVFGTGTKLTVL

SEQ ID NO: 23 - protein, 264 aa, Artificial Sequence
  anti-ASPH CAR antigen-binding fragment single chain Fv
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARTGYSSSWVVNFDYWGQGTLVTVSSGSASAPTGILGSGGGGSGGGGSGGGGSQPVLTQSPSASGTPGQRVTISCSGSSSNIGSNYVYWYQQLPGTAPKLLIYKNNQRPSGVPDRFSGSKSGTAASLAISGLQSEDEADYYCAAWDDSLRGYVFGTGTKLTVLSG

SEQ ID NO: 24 - protein, 259 aa, Artificial Sequence
  anti-ASPH CAR antigen-binding fragment single chain Fv
QVQLQQSGPGLVKPSQTLSLTCAISGDSVSSDSAAWNWIRQSPSRGLEWLGRTYYRSKWYNDYAVSVKSRISINPDTSKNQFSLQLNSVTPEDTAVYYCARAQNNIAVAGFDYWGLGTLVTVSSGILGSGGGGSGGGGSGGGGSQPVLTQSPSASGTPGQRVTISCSGSSSNIGSNYVYWYQQLPGTAPTLLIYRNNQRPSGVPDRFSGSKSGTSASLAISGLRSEDEAEYYCAAWDDSLSGLYVFGTGTKVTVLSGIL

SEQ ID NO: 25 - protein, 255 aa, Artificial Sequence
  anti-ASPH CAR antigen-binding fragment single chain Fv
QVQLQQSGAGLVKPSQTLSLTCTISGDSVSADRVAWNWIRQSPLRGLEWLGRIFYRSKWMVDYAVSVKSRISINPDTSKNQFSLQLNSVTPEDTAMYYCARATTRGYFDLWGRGTLVTVSSGILGSGGGGSGGGGSGGGGSQPVLTQSPSASGTPGQRVTISCSGSSSNIGSNYVYWYQQLPGTAPKLLIYKNNQRPSGVPDRFSGSKSGTAASLAISGLQSEDEADYYCAAWDDSLRGYVFGTGTKLTVLSGIL

SEQ ID NO: 26 - protein, 262 aa, Artificial Sequence
  anti-ASPH CAR antigen-binding fragment single chain Fv
QVQLQQSGPGLVKPSPTLSLTCAISGDSVSSNSAAWNWVRQSLSRGLEWLGRTYYRSKWYNGYAVSVRGRITTNADTSRNQFSLQLNSVTPEDTAVYYCARTGYSSSWVVNSNYWGQGTLVTVSSGSASAPTGILGSGGGGSGGGGSGGGGSQPALTQSPSASGTPGQRVTISCSGSSSNIGSNYVYWYQQLPGTAPKLLIYKNNQRPSGVPGRFSGSKSGTAASLAISGLRSKDEADYYCAAWDDSLRGYVFGTGTKLTVL

SEQ ID NO: 27 - protein, 22 aa, Artificial Sequence
  GMCSFalpha SP CAR construct component
MLLLVTSLLLCELPHPAFLLIP

SEQ ID NO: 28 - protein, 21 aa, Artificial Sequence
  CD8alpha SP CAR construct component
MALPVTALLLPLALLLHAARP

SEQ ID NO: 29 - protein, 69 aa, Artificial Sequence
  CD8alpha hinge/TM CAR construct component
TTTPAPRPPTPAPTIASQPLSLRPEACRPAAGGAVHTRGLDFACDIYIWAPLAGTCGVLLLSLVITLYC

SEQ ID NO: 30 - protein, 45 aa, Artificial Sequence
  CD8alpha hinge CAR construct component
TTTPAPRPPTPAPTIASQPLSLRPEACRPAAGGAVHTRGLDFACD

SEQ ID NO: 31 - protein, 24 aa, Artificial Sequence
  CD8alpha TM CAR construct component
IYIWAPLAGTCGVLLLSLVITLYC

SEQ ID NO: 32 - protein, 86 aa, Artificial Sequence
  CD28 hinge/TM/ Costim CAR construct component
VKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 33 - protein, 18 aa, Artificial Sequence
  CD28 hinge CAR construct component
VKGKHLCPSPLFPGPSKP

SEQ ID NO: 34 - protein, 27 aa, Artificial Sequence
  CD28 TM CAR construct component
FWVLVVVGGVLACYSLLVTVAFIIFWV

SEQ ID NO: 35 - protein, 41 aa, Artificial Sequence
  CD28 Costim CAR construct component
RSKRSRLLHSDYMNMTPRRPGPTRKHYQPYAPPRDFAAYRS

SEQ ID NO: 36 - protein, 42 aa, Artificial Sequence
  4-1BB costim domain CAR construct component
KRGRKKLLYIFKQPFMRPVQTTQEEDGCSCRFPEEEEGGCEL

SEQ ID NO: 37 - protein, 98 aa, Artificial Sequence
  CD3zeta signaling domain CAR construct component
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATK

SEQ ID NO: 38 - protein, 254 aa, Artificial Sequence
  GFP CAR construct component
SGMESDESGLPAMEIECRITGTLNGVEFELVGGGEGTPKQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRAQSSNSAVDGTAGPGSTGSR

SEQ ID NO: 39 - protein, 18 aa, Artificial Sequence
  T2A CAR construct component
EGRGSLLTCGDVEENPGP

SEQ ID NO: 40 - protein, 796 aa, Artificial Sequence
  BBz CAR construct 622-BBz
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARTGYSSSWVVNFDYWGQGTLVTVSSGSASAPTGILGSGGGGSGGGGSGGGGSQPVLTQSPSASGTPGQRVTISCSGSSSNIGSNYVYWYQQLPGTAPKLLIYKNNQRPSGVPDRFSGSKSGTAASLAISGLQSEDEADYYCAAWDDSLRGYVFGTGTKLTVLSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKRRKRGSGAAAEGRGSLLTCGDVEENPGPSGMESDESGLPAMEIECRITGTLNGVEFELVGGGEGTPKQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRAQSSNSAVDGTAGPGSTGSR

SEQ ID NO: 41 - protein, 513 aa, Artificial Sequence
  BBz CAR construct 622-BBzdeltaGFP
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARTGYSSSWVVNFDYWGQGTLVTVSSGSASAPTGILGSGGGGSGGGGSGGGGSQPVLTQSPSASGTPGQRVTISCSGSSSNIGSNYVYWYQQLPGTAPKLLIYKNNQRPSGVPDRFSGSKSGTAASLAISGLQSEDEADYYCAAWDDSLRGYVFGTGTKLTVLSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 792 aa, Artificial Sequence
  BBz CAR construct 623-BBz
MLLLVTSLLLCELPHPAFLLIPQVQLQQSGPGLVKPSQTLSLTCAISGDSVSSDSAAWNWIRQSPSRGLEWLGRTYYRSKWYNDYAVSVKSRISINPDTSKNQFSLQLNSVTPEDTAVYYCARAQNNIAVAGFDYWGLGTLVTVSSGILGSGGGGSGGGGSGGGGSQPVLTQSPSASGTPGQRVTISCSGSSSNIGSNYVYWYQQLPGTAPTLLIYRNNQRPSGVPDRFSGSKSGTSASLAISGLRSEDEAEYYCAAWDDSLSGLYVFGTGTKVTVLSGILGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKRRKRGSGAAAEGRGSLLTCGDVEENPGPSGMESDESGLPAMEIECRITGTLNGVEFELVGGGEGTPKQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRAQSSNSAVDGTAGPGSTGSR

SEQ ID NO: 43 - protein, 509 aa, Artificial Sequence
  BBz CAR construct 623-BBzdeltaGFP
MLLLVTSLLLCELPHPAFLLIPQVQLQQSGPGLVKPSQTLSLTCAISGDSVSSDSAAWNWIRQSPSRGLEWLGRTYYRSKWYNDYAVSVKSRISINPDTSKNQFSLQLNSVTPEDTAVYYCARAQNNIAVAGFDYWGLGTLVTVSSGILGSGGGGSGGGGSGGGGSQPVLTQSPSASGTPGQRVTISCSGSSSNIGSNYVYWYQQLPGTAPTLLIYRNNQRPSGVPDRFSGSKSGTSASLAISGLRSEDEAEYYCAAWDDSLSGLYVFGTGTKVTVLSGILGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 760 aa, Artificial Sequence
  BBz CAR construct LLm13-BBz
MLLLVTSLLLCELPHPAFLLIPQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSGSGGGGSGGGGSGGGGSSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGSVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKRRKRGSGAAAEGRGSLLTCGDVEENPGPSGMESDESGLPAMEIECRITGTLNGVEFELVGGGEGTPKQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRAQSSNSAVDGTAGPGSTGSR

SEQ ID NO: 45 - protein, 505 aa, Artificial Sequence
  BBz CAR construct LLm13-BBzdeltaGFP
MLLLVTSLLLCELPHPAFLLIPQVQLQQSGAGLVKPSQTLSLTCTISGDSVSADRVAWNWIRQSPLRGLEWLGRIFYRSKWMVDYAVSVKSRISINPDTSKNQFSLQLNSVTPEDTAMYYCARATTRGYFDLWGRGTLVTVSSGILGSGGGGSGGGGSGGGGSQPVLTQSPSASGTPGQRVTISCSGSSSNIGSNYVYWYQQLPGTAPKLLIYKNNQRPSGVPDRFSGSKSGTAASLAISGLQSEDEADYYCAAWDDSLRGYVFGTGTKLTVLSGILGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 770 aa, Artificial Sequence
  BBz CAR construct C4m18-BBz
MLLLVTSLLLCELPHPAFLLIPQVQLQQSGPGLVKPSPTLSLTCAISGDSVSSNSAAWNWVRQSLSRGLEWLGRTYYRSKWYNGYAVSVRGRITTNADTSRNQFSLQLNSVTPEDTAVYYCARTGYSSSWVVNSNYWGQGTLVTVSSGSASAPTGILGSGGGGSGGGGSGGGGSQPALTQSPSASGTPGQRVTISCSGSSSNIGSNYVYWYQQLPGTAPKLLIYKNNQRPSGVPGRFSGSKSGTAASLAISGLRSKDEADYYCAAWDDSLRGYVFGTGTKLTVLGGGGSVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKRRKRGSGAAAEGRGSLLTCGDVEENPGPSGMESDESGLPAMEIECRITGTLNGVEFELVGGGEGTPKQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRAQSSNSAVDGTAGPGSTGSR

SEQ ID NO: 47 - protein, 512 aa, Artificial Sequence
  BBz CAR construct C4m18-BBzdeltaGFP
MLLLVTSLLLCELPHPAFLLIPQVQLQQSGPGLVKPSPTLSLTCAISGDSVSSNSAAWNWVRQSLSRGLEWLGRTYYRSKWYNGYAVSVRGRITTNADTSRNQFSLQLNSVTPEDTAVYYCARTGYSSSWVVNSNYWGQGTLVTVSSGSASAPTGILGSGGGGSGGGGSGGGGSQPALTQSPSASGTPGQRVTISCSGSSSNIGSNYVYWYQQLPGTAPKLLIYKNNQRPSGVPGRFSGSKSGTAASLAISGLRSKDEADYYCAAWDDSLRGYVFGTGTKLTVLGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 770 aa, Artificial Sequence
  28z CAR construct 622-28z
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARTGYSSSWVVNFDYWGQGTLVTVSSGSASAPTGILGSGGGGSGGGGSGGGGSQPVLTQSPSASGTPGQRVTISCSGSSSNIGSNYVYWYQQLPGTAPKLLIYKNNQRPSGVPDRFSGSKSGTAASLAISGLQSEDEADYYCAAWDDSLRGYVFGTGTKLTVLSGGGGSVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKRRKRGSGAAAEGRGSLLTCGDVEENPGPSGMESDESGLPAMEIECRITGTLNGVEFELVGGGEGTPKQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRAQSSNSAVDGTAGPGSTGSR

SEQ ID NO: 49 - protein, 766 aa, Artificial Sequence
  28z CAR construct 623-28z
MALPVTALLLPLALLLHAARPQVQLQQSGPGLVKPSQTLSLTCAISGDSVSSDSAAWNWIRQSPSRGLEWLGRTYYRSKWYNDYAVSVKSRISINPDTSKNQFSLQLNSVTPEDTAVYYCARAQNNIAVAGFDYWGLGTLVTVSSGILGSGGGGSGGGGSGGGGSQPVLTQSPSASGTPGQRVTISCSGSSSNIGSNYVYWYQQLPGTAPTLLIYRNNQRPSGVPDRFSGSKSGTSASLAISGLRSEDEAEYYCAAWDDSLSGLYVFGTGTKVTVLSGILGGGGSVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKRRKRGSGAAAEGRGSLLTCGDVEENPGPSGMESDESGLPAMEIECRITGTLNGVEFELVGGGEGTPKQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRAQSSNSAVDGTAGPGSTGSR

SEQ ID NO: 50 - protein, 760 aa, Artificial Sequence; identical to SEQ ID NO: 44
  28z CAR construct LLm13-28z

SEQ ID NO: 51 - protein, 770 aa, Artificial Sequence; identical to SEQ ID NO: 46
  28z CAR construct C4m18-28z

SEQ ID NO: 52 - protein, 5 aa, Artificial Sequence
  linker peptide
DGGGS

SEQ ID NO: 53 - protein, 5 aa, Artificial Sequence
  linker peptide
TGEKP

SEQ ID NO: 54 - protein, 4 aa, Artificial Sequence
  linker peptide
GGRR

SEQ ID NO: 55 - protein, 25 aa, Artificial Sequence
  linker peptide
  MISC_FEATURE (6)..(10): May be absent
  MISC_FEATURE (11)..(15): May be absent
  MISC_FEATURE (16)..(20): May be absent
  MISC_FEATURE (21)..(25): May be absent
GGGGSGGGGSGGGGSGGGGSGGGGS

SEQ ID NO: 56 - protein, 14 aa, Artificial Sequence
  linker peptide
EGKSSGSGSESKVD

SEQ ID NO: 57 - protein, 18 aa, Artificial Sequence
  linker peptide
KESGSVSSEQLAQFRSLD

SEQ ID NO: 58 - protein, 8 aa, Artificial Sequence
  linker peptide
GGRRGGGS

SEQ ID NO: 59 - protein, 9 aa, Artificial Sequence
  linker peptide
LRQRDGERP

SEQ ID NO: 60 - protein, 12 aa, Artificial Sequence
  linker peptide
LRQKDGGGSERP

SEQ ID NO: 61 - protein, 16 aa, Artificial Sequence
  linker peptide
LRQKDGGGSGGGSERP

SEQ ID NO: 62 - protein, 18 aa, Artificial Sequence
  linker peptide
GSTSGSGKPGSGEGSTKG

SEQ ID NO: 63 - protein, 22 aa, Artificial Sequence
  exemplary 2A self-cleaving polypeptide site
GSGATNFSLLKQAGDVEENPGP

SEQ ID NO: 64 - protein, 19 aa, Artificial Sequence
  exemplary 2A self-cleaving polypeptide site
ATNFSLLKQAGDVEENPGP

SEQ ID NO: 65 - protein, 14 aa, Artificial Sequence
  exemplary 2A self-cleaving polypeptide site
LLKQAGDVEENPGP

SEQ ID NO: 66 - protein, 21 aa, Artificial Sequence
  exemplary 2A self-cleaving polypeptide site
GSGEGRGSLLTCGDVEENPGP

SEQ ID NO: 67 - protein, 13 aa, Artificial Sequence
  exemplary 2A self-cleaving polypeptide site
LLTCGDVEENPGP

SEQ ID NO: 68 - protein, 23 aa, Artificial Sequence
  exemplary 2A self-cleaving polypeptide site
GSGQCTNYALLKLAGDVESNPGP

SEQ ID NO: 69 - protein, 20 aa, Artificial Sequence
  exemplary 2A self-cleaving polypeptide site
QCTNYALLKLAGDVESNPGP

SEQ ID NO: 70 - protein, 14 aa, Artificial Sequence
  exemplary 2A self-cleaving polypeptide site
LLKLAGDVESNPGP

SEQ ID NO: 71 - protein, 25 aa, Artificial Sequence
  exemplary 2A self-cleaving polypeptide site
GSGVKQTLNFDLLKLAGDVESNPGP

SEQ ID NO: 72 - protein, 22 aa, Artificial Sequence
  exemplary 2A self-cleaving polypeptide site
VKQTLNFDLLKLAGDVESNPGP

SEQ ID NO: 73 - protein, 14 aa, Artificial Sequence
  exemplary 2A self-cleaving polypeptide site
LLKLAGDVESNPGP

SEQ ID NO: 74 - protein, 19 aa, Artificial Sequence
  exemplary 2A self-cleaving polypeptide site
LLNFDLLKLAGDVESNPGP

SEQ ID NO: 75 - protein, 19 aa, Artificial Sequence
  exemplary 2A self-cleaving polypeptide site
TLNFDLLKLAGDVESNPGP

SEQ ID NO: 76 - protein, 14 aa, Artificial Sequence
  exemplary 2A self-cleaving polypeptide site
LLKLAGDVESNPGP

SEQ ID NO: 77 - protein, 17 aa, Artificial Sequence
  exemplary 2A self-cleaving polypeptide site
NFDLLKLAGDVESNPGP

SEQ ID NO: 78 - protein, 20 aa, Artificial Sequence
  exemplary 2A self-cleaving polypeptide site
QLLNFDLLKLAGDVESNPGP

SEQ ID NO: 79 - protein, 24 aa, Artificial Sequence
  exemplary 2A self-cleaving polypeptide site
APVKQTLNFDLLKLAGDVESNPGP

SEQ ID NO: 80 - protein, 40 aa, Artificial Sequence
  exemplary 2A self-cleaving polypeptide site
VTELLYRMKRAETYCPRPLLAIHPTEARHKQKIVAPVKQT

SEQ ID NO: 81 - protein, 18 aa, Artificial Sequence
  exemplary 2A self-cleaving polypeptide site
LNFDLLKLAGDVESNPGP

SEQ ID NO: 82 - protein, 40 aa, Artificial Sequence
  exemplary 2A self-cleaving polypeptide site
LLAIHPTEARHKQKIVAPVKQTLNFDLLKLAGDVESNPGP

SEQ ID NO: 83 - protein, 33 aa, Artificial Sequence
  exemplary 2A self-cleaving polypeptide site
EARHKQKIVAPVKQTLNFDLLKLAGDVESNPGP

(19) United States
(12) Patent Application Publication
(10) Pub. No.: US 2019/0345261 A1
(43) Pub. Date: Nov. 14, 2019

(54) ASPARTATE BETA-HYDROXYLASE CHIMERIC ANTIGEN RECEPTORS AND USES THEREOF

(71) Applicant: SENSEI BIOTHERAPEUTICS, INC., Gaithersburg, MD (US)

(72) Inventors: Michael S. Lebowitz, Pikesville, MD (US); Hossein A. Ghanbari, Potomac, MD (US); Thomas Thisted, New Market, MD (US); Zhi-Gang Jiang, Gaithersburg, MD (US)

(21) Appl. No.: 16/409,250

(22) Filed: May 10, 2019

Related U.S. Application Data
(60) Provisional application No. 62/811,223, filed on Feb. 27, 2019.
(60) Provisional application No. 62/669,752, filed on May 10, 2018.

(51) Int. Cl. C07K 16/40; C12N 5/0783; A61P 35/00; A61K 35/17; A61K 45/06; A61K 39/395; A61K 38/17; C07K 14/705; C07K 14/725; C07K 14/73

(52) U.S. Cl. CPC C07K 16/40; C07K 2317/92; A61P 35/00; A61K 35/17; A61K 45/06; A61K 39/3955; A61K 38/1774; A61K 38/177; C07K 14/70521; C07K 14/70578; C07K 14/7051; C07K 14/70514; C07K 14/70517; C07K 2319/30; C07K 2319/03; C07K 2319/02; C07K 2319/33; C07K 2317/76; C07K 2317/565; C07K 2317/622; C07K 2317/33; C07K 2317/53; C12N 2510/00; C07K 2317/732; C07K 2317/734; C12N 5/0636

(57) ABSTRACT

Provided herein are anti-ASPH chimeric antigen receptors (CARs), genetically modified immune effector cells, and use of these compositions to effectively treat ASPH expressing cancers.

### CROSS-REFERENCE TO RELATED APPLICATIONS

This application claims priority to U.S. Provisional Patent Application No. 62/811,223, filed on Feb. 27, 2019 and U.S. Provisional Patent Application No. 62/669,752, filed on May 10, 2018. The disclosures of each of these applications is incorporated herein by reference in its entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: SEBI_009_001US_SeqList_ST25.txt, date recorded: May 10, 2019, file size 110,953 bytes).

### FIELD

The present invention relates to compositions and methods for treating ASPH-expressing cancer. More particularly, the invention relates to anti-ASPH chimeric antigen receptors (CARs), genetically modified immune effector cells, and use of these compositions to effectively treat ASPH expressing cancers.

### BACKGROUND

Aspartate β-hydroxylase (ASPH) is a type II transmembrane protein predominantly expressed during embryogenesis, where it promotes cell migration for organ development. ASPH has very low expression in healthy adult tissue, and is localized to the intracellular compartment of the endoplasmic reticulum. However, re-expression and translocation to the tumor cell surface has been detected in more than 20 different types of cancers including lung, liver, colon, pancreas, prostate, ovary, bile duct, and breast cancers, with expression levels inversely correlated with disease prognosis (Yeung et al., (2007) Human Antibodies, 16, 163-176). Additional immunotherapeutic approaches targeting ASPH are needed to treat cancer.

### SUMMARY

Provided herein is a chimeric antigen receptor (CAR) comprising: (a) an extracellular domain comprising an antigen-binding domain that specifically binds to human aspartate β-hydroxylase (ASPH); (b) a transmembrane region; and (c) an intracellular domain.

In some embodiments, the antigen-binding domain of an anti-ASPH CAR is an antigen-binding fragment of an anti-ASPH antibody. In some embodiments, the antigen-binding fragment comprises a heavy chain variable (VH) region comprising the CDRH1, CDRH2 and CDRH3 and a light chain variable (VL) region comprising the CDRL1, CDRL2 and CDRL3 of an anti-ASPH antibody. In some embodiments, the antigen-binding fragment comprises:

(a) the CDRH1 of SEQ ID NO: 1, the CDRH2 of SEQ ID NO: 2, and the CDRH3 of SEQ ID NO: 3; and the CDRL1 of SEQ ID NO: 4, the CDRL2 of SEQ ID NO: 5, the CDRL3 of SEQ ID NO: 6;

(b) the CDRH1 of SEQ ID NO: 7, the CDRH2 of SEQ ID NO: 2, and the CDRH3 of SEQ ID NO: 8; and the CDRL1 of SEQ ID NO: 4, the CDRL2 of SEQ ID NO: 9, the CDRL3 of SEQ ID NO: 10;

(c) the CDRH1 of SEQ ID NO: 11, the CDRH2 of SEQ ID NO: 12, and the CDRH3 of SEQ ID NO: 13; and the CDRL1 of SEQ ID NO: 4, the CDRL2 of SEQ ID NO: 5, the CDRL3 of SEQ ID NO: 6; or

(d) the CDRH1 of SEQ ID NO: 1, the CDRH2 of SEQ ID NO: 14, and the CDRH3 of SEQ ID NO: 15; and the CDRL1 of SEQ ID NO: 4, the CDRL2 of SEQ ID NO: 5, the CDRL3 of SEQ ID NO: 6.

Further provided herein is an anti-ASPH CAR comprising an antigen-binding fragment that comprises:

(a) the VH region comprises the amino acid sequence of SEQ ID NO: 16 and the VL region comprises the amino acid sequence of SEQ ID NO: 17;

(b) the VH region comprises the amino acid sequence of SEQ ID NO: 18 and the VL region comprises the amino acid sequence of SEQ ID NO: 19;

(c) the VH region comprises the amino acid sequence of SEQ ID NO: 20 and the VL region comprises the amino acid sequence of SEQ ID NO: 17; or

(d) the VH region comprises the amino acid sequence of SEQ ID NO: 21 and the VL region comprises the amino acid sequence of SEQ ID NO: 22.

In some embodiments, the antigen-binding fragment of an anti-ASPH CAR is a single chain Fv (scFv). In some embodiments, the scFv comprises an amino acid sequence selected from SEQ ID NOs: 23-26.

In some embodiments, the antigen-binding domain of an anti-ASPH CAR is a Fibronectin type III domain, a Tn3 protein, a designed ankyrin repeat protein, an affibody, a camelid nanobody, a shark antibody domain, an anticalin, an anti-ASPH aptamer, an EGF-like domain, a human Notch receptor derivative or a human Notch ligand derivative.

Further provided herein is an anti-ASPH CAR comprising an antigen-binding domain that cross-competes for binding to ASPH (e.g., human ASPH) with an anti-ASPH antibody comprising:

(a) a VH region comprising the amino acid sequence of SEQ ID NO: 16 and a VL region comprising the amino acid sequence of SEQ ID NO: 17;

(b) a VH region comprising the amino acid sequence of SEQ ID NO: 18 and a VL region comprising the amino acid sequence of SEQ ID NO: 19;

(c) a VH region comprising the amino acid sequence of SEQ ID NO: 20 and a VL region comprising the amino acid sequence of SEQ ID NO: 17; or

(d) a VH region comprising the amino acid sequence of SEQ ID NO: 21 and a VL region comprising the amino acid sequence of SEQ ID NO: 22.

Also provided herein is an anti-ASPH CAR comprising an extracellular domain that further comprises a signal peptide. In some embodiments, the signal peptide comprises a sequence encoding a human CD2, CD3δ, CD3ε, CD3γ, CD3ζ, CD4, CD8α, CD19, CD28, CD37, CD45, 4-1BB, GM-CSFR, IL-2, CD33, Human IgKVIII, Human IgG2 H, Chymotrypsinogen, trypsinogen-2, HSA, Insulin or tPA signal peptide.

Also provided herein is an anti-ASPH CAR comprising an extracellular domain that further comprises an additional antigen-binding domain that specifically binds to an antigen other than ASPH. In some embodiments, the additional antigen-binding domain specifically binds to CD19, CD20, CD22, CD5, CD123, CD33, CD70, CD38, CD133, CD138, BCMA (B cell maturation antigen), Mesothelin, GPC3, EpCam, Her2, Muc1, PSCA, CEA, ROR1, GAP, Pan-ErbB, GD2, EphA2, EGFRVIII, IL13Rα2, PSMA, VEGFR2, mucin 16, Lewis-Y or immunoglobulin kappa light chain.

Also provided herein is an anti-ASPH CAR comprising an extracellular domain that further comprises a hinge region. In some embodiments, the hinge region comprises a sequence derived from a human CD8α, IgG4, CD28, and/or CD4 sequence.

Further provided herein is an anti-ASPH CAR, wherein the transmembrane region comprises a sequence encoding a human CD2, CD3δ, CD3ε, CD3γ, CD3ζ, CD4, CD8α, CD19, CD28, CD37, CD45, 4-1BB or GM-CSFR transmembrane domain.

Also contemplated herein is an anti-ASPH CAR comprising an intracellular domain comprising a signaling domain. In some embodiments, the signaling domain comprises one or more of an intracellular signaling portion of human CD3 zeta, CD28, CD137, TCR zeta, FcR gamma, FcR beta, CD3 gamma, CD3 delta or CD3 epsilon.

In some embodiments, an anti-ASPH CAR comprises an intracellular domain that comprises one, two or three costimulatory domains selected from a human 4-1BB, CD28, CD2, CD27, CD30, CD40, CD40LG (CD40L), DAP-12, OX40, inducible T cell costimulator (ICOS), MyD88, KLRC2 (NKG2C), TNFRS18 (GITR), TNFRSF14 (HVEM), or ITGB2 (LFA-1) costimulatory domain.

In some embodiments, an anti-ASPH CAR comprises an intracellular domain that comprises a signaling domain adjacent to a costimulatory domain.

Further provided herein is a CAR comprising an amino acid sequence selected from SEQ ID NOs: 40-51.

Also contemplated herein is a nucleic acid molecule comprising a nucleotide sequence encoding a CAR as disclosed herein. In some embodiments, such a nucleic acid molecule, further comprises a suicide gene-encoding nucleotide sequence upstream or downstream of the nucleotide sequence encoding the CAR. In some embodiments, the suicide gene-encoding nucleotide sequence encodes an inducible human caspase. In some embodiments, the inducible human caspase is inducible human caspase-9 (iCasp9).

In some embodiments, a nucleic acid molecule provided herein further encodes a cytokine. In some embodiments, the cytokine is IL-15, IL-7, IL-12 or IL-21. In some embodiments, a nucleic acid molecule provided herein further encodes a costimulatory molecule. In some embodiments, the costimulatory molecule is CD40-L or 4-1BB-L. In some embodiments, a nucleic acid molecule provided herein further encodes a degrading enzyme.

Further provided herein is a cell comprising a CAR as disclosed herein. In some aspects, the cell expresses the CAR on the cell surface. In some embodiments, the cell is an immune effector cell. For example, the immune effector cell may be, without limitation, a T-cell, a Natural Killer (NK) cell, a Natural Killer (NK)-like cell, a hematopoietic progenitor cell, a peripheral blood (PB) derived T cell or an umbilical cord blood (UCB) derived T-cell.

In some embodiments, a cell comprising a CAR further expresses an inhibitor of an immune checkpoint molecule. In some embodiments, the immune checkpoint molecule is PD1, PD-L1, PD-L2, CTLA4, TIM3, CEACAM-1, CEACAM-3, CEACAM-5, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine or TGFR.

In some embodiments, a cell comprising a CAR further expresses a PD-1 dominant negative receptor.

In some embodiments, a cell comprising a CAR further expresses an apoptosis-inducing agent. In some embodiments, the apoptosis-inducing agent is a TRAILR2 agonist.

Further disclosed herein is a composition comprising a cell comprising a CAR provided herein.

Also provided herein is a method for expressing a chimeric antigen receptor (CAR) on the surface of a cell, comprising:

(a) obtaining a cell population;

(b) contacting the cell population with a composition comprising a CAR disclosed herein or a nucleic acid molecule encoding the CAR, under conditions sufficient to transfer the CAR across a cell membrane of at least one cell in the cell population, thereby generating a modified cell population;

(c) culturing the modified cell population under conditions suitable for integration of the CAR; and

(d) expanding and/or selecting at least one cell from the modified cell population that express the CAR on the cell surface.

Further provided herein is a method for treating cancer in a subject, comprising administering to the subject an effective amount of cells comprising a CAR as disclosed herein. Also provided herein is a method for ameliorating at least one symptom of cancer in a subject, comprising administering to the subject an effective amount of cells comprising a CAR as disclosed herein. In some embodiments, the cell is an allogeneic cell. In some embodiments, the cell is an autologous cell. In some embodiments, the cancer is ASPH-expressing cancer. In some embodiments, the cancer is a solid tumor or a hematological malignancy. In some embodiments, the cancer is prostate, liver, bile duct, brain, head-and-neck, breast, colon, ovarian, cervical, pancreatic or lung cancer. In some embodiments, the method further comprises administering a chemotherapeutic agent, radiation and/or an allogeneic stem cell transplant to the subject. In some embodiments, the cell comprising an anti-ASPH CAR is engineered to be immune to the chemotherapeutic agent. In some embodiments, the method further comprises administering an inhibitor of an immune checkpoint molecule to the subject. In some embodiments, the immune checkpoint molecule is PD1, PD-L1, PD-L2, CTLA4, TIM3, CEACAM-1, CEACAM-3, CEACAM-5, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MEW class I, MHC class II, GALS, adenosine, or TGFR.

Also provided herein is a cell comprising an anti-ASPH CAR as disclosed herein or a composition as disclosed herein, for use as a medicament.

### BRIEF DESCRIPTION OF THE DRAWINGS

FIG. 1 shows illustrative schematics of the anti-ASPH CAR constructs described herein.

FIG. 2A-FIG. 2E show expression of 28z and BBz CAR constructs on the surface of HT-1080 cells. FIG. 2A shows expression of 622-28z and 622-BBz constructs. FIG. 2B shows expression of 623-28z and 623-BBz constructs. FIG. 2C shows expression of LLm13-28z and LLm13-BBz constructs. FIG. 2D shows expression of C4m18-28z and C4m18-BBz constructs.

FIG. 2E shows expression of anti-FITC-28z and -BBz control constructs.

FIG. 3A-FIG. 3B show analysis of T cell populations before (FIG. 3A) and after (FIG. 3B) T cell isolation.

FIG. 4 shows expression of CAR constructs on the cell surface of transduced primary human T cells.

FIG. 5A-FIG. 5C show cell surface expression of BBz vs. BBzΔGFP CAR constructs. FIG. 5A compares the original FITC construct to FITCAGFP. FIG. 5B compares 622-BBz to 622-BBzΔGFP. FIG. 5C shows 622-BBzΔGFP, 623-BBzΔGFP, LLm13-BBzΔGFP and FITC-BBzΔGFP. In each panel, uninfected T-cells were stained in parallel as controls (marked with a “C”).

FIG. 6A-FIG. 6D show the effect of employing the SFFV promoter to drive CAR expression. FIG. 6A shows direct comparison of the 3 successive generations of the 622-BBz CAR constructs: the original 622-BBz (co-expressing GFP, labelled “Original”), 622-BBzΔGFP expressed from the EF1α promoter (labelled “AGFP”), and the 622-BBzΔGFP CAR expressed from the SFFV promoter (labelled “SFFV”). FIG. 6B shows a comparison of the 623-BBzΔGFP construct expressed from the EF1α promoter or the SFFV promoter. FIG. 6C shows a comparison of the LLm13-BBzΔGFP construct expressed from the EF1α promoter or the SFFV promoter.

FIG. 6D shows expression of the control FITC construct from the SFFV promoter. In each panel, uninfected T-cells were stained in parallel as controls (marked with a “C”).

FIG. 7 shows LDH activity levels from a co-culture experiment with ASPH-expressing H460 target cells and ASPH CAR-T cells.

FIG. 8 shows IFNγ levels from a co-culture experiment with ASPH-expressing H460 target cells and ASPH CAR-T cells.

FIG. 9A-FIG. 9C show the effects of ASPH CAR-T cells in co-culture experiments with ASPH-expressing H460 target cells. FIG. 9A is a graph showing CAR expression levels on transduced and expanded T-cells. FIG. 9B shows the cell killing activity of CAR-T cells. FIG. 9C is a bar graph showing levels of IFNγ released in the co-culture experiments.

FIG. 10A-FIG. 10B show the effects of ASPH CAR-T cells in co-culture experiments with ASPH-expressing MOLM-14 and Raji target cells. FIG. 10A shows levels of ASPH expression in MOLM-14, Raji and H460 cells as determined by flow cytometry. FIG. 10B shows the flow analysis and the 3 gates (identical for all 5 data sets) delineating T-cells, MOLM-14 and Raji populations, respectively (the t=0 samples for the LLm13 co-culture were not analyzed).

FIG. 11A-FIG. 11E show the effects of ASPH CAR-T cells in co-culture experiments with ASPH-expressing H460 target cells and Normal Human Lung Fibroblasts (NHLF). FIG. 11A is a graph showing CAR expression levels on transduced and expanded T-cells. FIG. 11B shows the cell killing activity of 622-BBz-SFFV CAR-T cells. FIG. 11C is a bar graph showing levels of IFNγ released in the co-culture experiments. FIG. 11D show images comparing ASPH expression on H460 cells and NHLF cells. FIG. 11E is a bar graph comparing cell killing activity of T-cells expressing the 622-BBz-SFFV CAR on H460 cells (on the left at each time point) and NHLF cells (on the right at each time point).

### DETAILED DESCRIPTION

### Overview

The present disclosure generally relates to compositions and methods for preventing or treating cancers that express ASPH or preventing, treating, or ameliorating at least one symptom associated with an ASPH expressing cancer. In particular embodiments, the invention relates to adoptive cell therapy of cancers that express ASPH using genetically modified immune effector cells. Genetic approaches offer a potential means to enhance immune recognition and elimination of cancer cells. One promising strategy is to genetically engineer immune effector cells to express chimeric antigen receptors (CAR) that redirect cytotoxicity toward cancer cells.

The compositions and methods of adoptive cell therapy contemplated herein provide genetically modified immune effector cells that demonstrate antigen dependent cytotoxicity to cells expressing human (HAA) aspartate β-hydroxylase (ASPH) (NCBI Gene ID: 444), also known as aspartyl/asparaginyl beta-hydroxylase (AAH), and aspartyl (asparaginyl) β-hydroxylase (HAAH).

Illustrative examples of polynucleotide sequences encoding ASPH include, but are not limited to: NM_001164750.1, and NM_004318.4. Illustrative examples of polypeptide sequences encoding ASPH include, but are not limited to: NP_001158222.1, and NP_004309.2.

In one embodiment, a CAR comprising an extracellular domain comprising an antigen-binding domain that specifically binds to ASPH, a transmembrane domain, and an intracellular domain is provided.

In one embodiment, an immune effector cell is genetically modified to express an anti-ASPH CAR.

In various embodiments, genetically modified immune effector cells are administered to a subject with cancer cells expressing ASPH including, but not limited to, solid tumors and hematological malignancies.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir andCC Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below. Additional definitions are set forth throughout this disclosure.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

A numerical range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

### Chimeric Antigen Receptors

In various embodiments, genetically engineered receptors that redirect cytotoxicity of immune effector cells toward cancer cells expressing human aspartyl (asparaginyl) β-hydroxylase (ASPH) are provided herein. These genetically engineered receptors are referred to herein as chimeric antigen receptors (CARs). CARs are artificially constructed hybrid proteins or polypeptides that combine binding specificity for a desired antigen (e.g., ASPH) with a T cell receptor-activating intracellular domain to generate a chimeric protein that exhibits a specific anti-ASPH cellular immune activity and activate the T cell upon interaction with the target antigen (e.g., ASPH).

In some embodiments, CARs comprise an extracellular domain (comprising a binding domain or antigen-specific binding domain) that binds to ASPH, a transmembrane domain, and an intracellular domain. In some embodiments, a CAR comprises, in amino-terminal to carboxyl-terminal order (a) an extracellular domain that binds to ASPH, (b) a transmembrane domain, and (c) an intracellular domain. Engagement of the anti-ASPH antigen binding domain of the CAR with ASPH on the surface of a target cell delivers an activation stimulus to the CAR-expressing cell. In some embodiments, engagement of the anti-ASPH antigen binding domain of the CAR with ASPH on the surface of a target cell results in clustering of the CAR and the subsequent activation of the CAR-expressing cell.

The main characteristic of CARs is their ability to exploiting the cell specific targeting abilities of monoclonal antibodies, soluble ligands or cell specific co-receptors by redirecting immune effector cell specificity, thereby triggering proliferation, cytokine production, phagocytosis, and/or production of molecules that can mediate cell death of the target antigen expressing cell in a major histocompatibility (WIC) independent manner. The non-MI-IC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

### Binding Domain

In particular embodiments, CARs comprise an extracellular domain that comprises an antigen-binding domain that specifically binds to ASPH (e.g., human ASPH). For example, the ASPH may be a human ASPH polypeptide expressed on a target cell, e.g., a cancer cell. In some embodiments, a CAR antigen-binding domain is an anti-ASPH antibody or antigen-binding fragment thereof. As used herein, the terms, “binding domain,” “antigen-binding domain,” “extracellular domain,” “extracellular binding domain,” “antigen-specific binding domain,” and “extracellular antigen specific binding domain,” are used interchangeably and provide a CAR with the ability to specifically bind to the target antigen of interest, e.g., ASPH. The binding domain may be derived either from a natural, synthetic, semi-synthetic, or recombinant source.

The terms “specific binding affinity” or “specifically binds” or “specifically bound” or “specific binding” or “specifically targets” as used herein, describe binding of an anti-ASPH antibody or antigen binding fragment thereof (or a CAR comprising the same) to ASPH at greater binding affinity than background binding. A binding domain (or a CAR comprising a binding domain or a fusion protein containing a binding domain) “specifically binds” to an ASPH polypeptide if it binds to or associates with ASPH with an affinity or Kₐ (i.e., an equilibrium association constant of a particular binding interaction with units of 1/M) of, for example, greater than or equal to about 10⁵ M⁻¹. In certain embodiments, a binding domain (or a fusion protein thereof) binds to a target with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains (or single chain fusion proteins thereof) refers to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater.

Alternatively, affinity may be defined as an equilibrium dissociation constant (K_{d}) of a particular binding interaction with units of M (e.g., 10⁻⁵ M to 10⁻¹³ M, or less). Affinities of binding domain polypeptides and CAR proteins according to the present disclosure can be readily determined using conventional techniques, e.g., by competitive ELISA (enzyme-linked immunosorbent assay), or by binding association, or displacement assays using labeled ligands, or using a surface-plasmon resonance device such as the BIACORE® T100, which is available from Biacore, Inc., Piscataway, N.J., or optical biosensor technology such as the EPIC system or EnSpire that are available from Corning and Perkin Elmer respectively (see also, e.g., Scatchard et al. (1949) Ann. N.Y. Acad. Sci. 51:660; and U.S. Pat. Nos. 5,283,173; 5,468,614, or the equivalent).

In some embodiments, a CAR antigen-binding domain may cross-compete for binding to ASPH (e.g., human ASPH) with an anti-ASPH antibody comprising a set of the VH and VL amino acid sequences provided in Table 2 (e.g., antibody 622, 623, LLm13 or C4m18). In some aspects, “cross-competes” means the ability of an antibody, antibody fragment or other antigen-binding domain to interfere with the binding of other antibodies, antibody fragments or antigen-binding domains to a specific antigen (e.g., human ASPH) in a standard competitive binding assay. The ability or extent to which an antibody, antibody fragment or other antigen-binding domain is able to interfere with the binding of another antibody, antibody fragment or antigen-binding domain to a specific antigen, and, therefore whether it can be said to cross-compete according to the invention, can be determined using standard competition binding assays. One suitable assay involves the use of the BIACORE® technology (e.g., by using the BIACORE® 3000 instrument (Biacore, Uppsala, Sweden)), which can measure the extent of interactions using surface plasmon resonance technology. Another assay for measuring cross-competing uses an ELISA-based approach. A high throughput process for “epitope binning” antibodies based upon their cross-competition is described in WO 2003/48731. Cross-competition is present if the antigen-binding domain under investigation reduces the binding of one of the antibodies described in Table 2 to human ASPH by 60% or more, specifically by 70% or more and more specifically by 80% or more and if one of the antibodies described in Table 2 reduces the binding of said antigen-binding domain to human ASPH by 60% or more, specifically by 70% or more and more specifically by 80% or more.

In particular embodiments, the extracellular binding domain of a CAR comprises an antibody or antigen binding fragment thereof. An “antibody” refers to a binding agent that is a polypeptide comprising at least a light chain or heavy chain immunoglobulin variable region which specifically recognizes and binds an epitope of an antigen, such as a peptide, lipid, polysaccharide, or nucleic acid containing an antigenic determinant, such as those recognized by an immune cell. The term also includes genetically engineered forms such as chimeric antibodies (for example, humanized murine antibodies), heteroconjugate antibodies (such as, bispecific antibodies) and antigen binding fragments thereof. See also, Pierce Catalog and Handbook, 1994-1995 (Pierce Chemical Co., Rockford, Ill.); Kuby, J., Immunology, 3_{rd} Ed., W. H. Freeman & Co., New York, 1997.

An “antigen (Ag)” refers to a compound, composition, or substance that can stimulate the production of antibodies or a T cell response in an animal, including compositions (such as one that includes a cancer-specific protein) that are injected or absorbed into an animal. An antigen reacts with the products of specific humoral or cellular immunity, including those induced by heterologous antigens, such as the disclosed antigens. In particular embodiments, the target antigen is an epitope of an ASPH polypeptide.

An “epitope” or “antigenic determinant” refers to the region of an antigen to which a binding agent binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs (which generally confer antigen binding) from another species, such as a mouse. In some embodiments, a CAR comprises antigen-specific binding domain that is a chimeric antibody or antigen binding fragment thereof.

In some embodiments, the antibody is a human antibody (such as a human monoclonal antibody) or fragment thereof that specifically binds to a human ASPH polypeptide. Human antibodies can be constructed by combining Fv clone variable domain sequence(s) selected from human-derived phage display or yeast display libraries with known human constant domain sequences(s) as described above. Alternatively, human monoclonal antibodies may be made by the hybridoma method. Human myeloma and mouse-human heteromyeloma cell lines for the production of human monoclonal antibodies have been described, for example, by Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991). In addition, transgenic animals (e.g., mice) can be used to produce a full repertoire of human antibodies in the absence of endogenous immunoglobulin production. See, e.g., Jakobovits et al., PNAS USA, 90: 2551 (1993); Jakobovits et al., Nature, 362: 255 (1993); Bruggermann et al., Year in Immunol., 7: 33 (1993). Gene shuffling can also be used to derive human antibodies from non-human, e.g., rodent antibodies, where the human antibody has similar affinities and specificities to the starting non-human antibody. (See WO 93/06213). Unlike traditional humanization of non-human antibodies by CDR grafting, this technique provides completely human antibodies, which have no FR or CDR residues of non-human origin.

In one embodiment, a CAR comprises a “humanized” antibody. A humanized antibody is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. Humanized or other monoclonal antibodies can have additional conservative amino acid substitutions, which have substantially no effect on antigen binding or other immunoglobulin functions. Humanized antibodies can be constructed by means of genetic engineering (see for example, U.S. Pat. No. 5,585,089).

Antigen binding fragments include Camel Ig, Ig NAR, Fab fragments, Fab′ fragments, F(ab′)2 fragments, bispecific Fab dimers (Fab2), trispecific Fab trimers (Fab3), Fv, single chain Fv proteins (“scFv”), bis-scFv, (scFv)₂, minibodies, diabodies, triabodies, tetrabodies, disulfide stabilized Fv proteins (“dsFv”), and single-domain antibody (sdAb, Nanobody) and portions of full length antibodies responsible for antigen binding. An “isolated antibody or antigen binding fragment thereof” is one which has been identified and separated and/or recovered from a component of its natural environment.

As would be understood by the skilled person and as described elsewhere herein, a complete antibody comprises two heavy chains and two light chains. Each heavy chain consists of a variable region and a first, second, and third constant region, while each light chain consists of a variable region and a constant region. Mammalian heavy chains are classified as α, δ, ε, γ, and μ. Mammalian light chains are classified as λ or κ. Immunoglobulins comprising the α, δ, ε, γ, and μ heavy chains are classified as immunoglobulin (Ig)A, IgD, IgE, IgG, and IgM. The complete antibody forms a “Y” shape. The stem of the Y consists of the second and third constant regions (and for IgE and IgM, the fourth constant region) of two heavy chains bound together and disulfide bonds (inter-chain) are formed in the hinge. Heavy chains γ, α and δ have a constant region composed of three tandem (in a line) Ig domains, and a hinge region for added flexibility; heavy chains μ and ε have a constant region composed of four immunoglobulin domains. The second and third constant regions are referred to as “CH2 domain” and “CH3 domain”, respectively. Each arm of the Y includes the variable region and first constant region of a single heavy chain bound to the variable and constant regions of a single light chain. The variable regions of the light and heavy chains are responsible for antigen binding.

Light and heavy chain variable regions contain a “framework” region interrupted by three hypervariable regions, also called “complementarity-determining regions” or “CDRs.” The CDRs can be defined or identified by conventional methods, such as by sequence according to Kabat et al. (Wu, T T and Kabat, E. A., J Exp Med. 132(2):211-50, (1970); Borden, P. and Kabat E. A., PNAS, 84: 2440-2443 (1987); (see, Kabat et al., Sequences of Proteins of Immunological Interest, U.S. Department of Health and Human Services, 1991, which is hereby incorporated by reference), or by structure according to Chothia et al (Chothia, C. and Lesk, A. M., J Mol. Biol., 196(4): 901-917 (1987), Chothia, C. et al, Nature, 342: 877-883 (1989)).

The sequences of the framework regions of different light or heavy chains are relatively conserved within a species, such as humans. The framework region of an antibody, that is the combined framework regions of the constituent light and heavy chains, serves to position and align the CDRs in three-dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, the CDRs located in the variable domain of the heavy chain of the antibody are referred to as CDRH1, CDRH2, and CDRH3, whereas the CDRs located in the variable domain of the light chain of the antibody are referred to as CDRL1, CDRL2, and CDRL3. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). Illustrative examples of light chain CDRs that are suitable for constructing anti-ASPH CARs contemplated in particular embodiments include, but are not limited to, the CDR sequences set forth in SEQ ID NOs: 4, 5, 6, 9, 10. Illustrative examples of heavy chain CDRs that are suitable for constructing anti-ASPH CARs contemplated in particular embodiments include, but are not limited to the CDR sequences set forth in SEQ ID NOs: 1, 2, 3, 7, 8, 11, 12, 13, 14, 15.

References to “VL” or “VL” refer to the variable region of an immunoglobulin light chain, including that of an antibody, Fv, scFv, dsFv, Fab, or other antibody fragment as contemplated herein. Illustrative examples of light chain variable regions that are suitable for constructing anti-ASPH CARs contemplated in particular embodiments include, but are not limited to, the light chain variable region sequences set forth in SEQ ID NOs:17, 19, and 22.

References to “VH” or “VH” refer to the variable region of an immunoglobulin heavy chain, including that of an antibody, Fv, scFv, dsFv, Fab, or other antibody fragment as contemplated herein. Illustrative examples of heavy chain variable regions that are suitable for constructing anti-ASPH CARs contemplated in particular embodiments include, but are not limited to, the heavy chain variable region sequences set forth in SEQ ID NOs: 16, 18, 20, and 21.

In particular embodiments, an anti-ASPH antibody or antigen binding fragment thereof, includes but is not limited to a Camel Ig (a camelid antibody (VHH)), Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), single-domain antibody (sdAb, Nanobody) and a shark antibody domain.

“Camel Ig” or “camelid VHH” as used herein refers to the smallest known antigen-binding unit of a heavy chain antibody (Koch-Nolte, et al, FASEB J., 21: 3490-3498 (2007)). A “heavy chain antibody” or a “camelid antibody” refers to an antibody that contains two VH domains and no light chains (Riechmann L. et al, J. Immunol. Methods 231:25-38 (1999); WO94/04678; WO94/25591; U.S. Pat. No. 6,005,079). In some embodiments, an antigen-binding domain is a camelid nanobody.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three hypervariable regions (HVRs) of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known. Bispecific Fab dimers (Fab2) have two Fab′ fragments, each binding a different antigen. Trispecific Fab trimers (Fab3) have three Fab′ fragments, each binding a different antigen.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., PNAS USA 90: 6444-6448 (1993). Triabodies and tetrabodies are also described in Hudson et al., Nat. Med. 9:129-134 (2003).

“Single domain antibody” or “sdAb” or “nanobody” refers to an antibody fragment that consists of the variable region of an antibody heavy chain (VH domain) or the variable region of an antibody light chain (VL domain) (Holt, L., et al, Trends in Biotechnology, 21(11): 484-490).

“Single-chain Fv” or “scFv” antibody fragments comprise the VH and VL domains of antibody, wherein these domains are present in a single polypeptide chain and in either orientation (e.g., VL-VH or VH-VL). Generally, the scFv polypeptide further comprises a polypeptide linker between the VH and VL domains which enables the scFv to form the desired structure for antigen binding. For a review of scFv, see, e.g., Pluckthün, in The Pharmacology of Monoclonal Antibodies, vol. 113, Rosenburg and Moore eds., (Springer-Verlag, New York, 1994), pp. 269-315.

In some embodiments, the anti-ASPH antigen binding fragment is an scFv. In particular embodiments, the scFv is a murine, human or humanized scFv. Single chain antibodies may be cloned form the V region genes of a hybridoma specific for a desired target. The production of such hybridomas has become routine. A technique which can be used for cloning the variable region heavy chain (V_{H}) and variable region light chain (V_{L}) has been described, for example, in Orlandi et al., PNAS, 1989; 86: 3833-3837.

In some embodiments, a CAR antigen-binding domain comprises a heavy chain variable (VH) region comprising the CDRH1, CDRH2 and CDRH3 and a light chain variable (VL) region comprising the CDRL1, CDRL2 and CDRL3 of an anti-ASPH antibody. In some embodiments, an anti-ASPH antibody or antigen binding fragment thereof comprises a variable light chain sequence comprising an LCDR1 sequence set forth in SEQ ID NO: 4, an LCDR2 sequence set forth in one of SEQ ID NOs: 5 and 9, and an LCDR3 sequence set forth in one of SEQ ID NOs: 6 and 10. In some embodiments, an anti-ASPH antibody or antigen binding fragment thereof comprises a variable heavy chain sequence comprising an HCDR1 sequence set forth in one of SEQ ID NOs: 1, 7, and 11, an HCDR2 sequence set forth in one of SEQ ID NOs: 2, 12, and 14, and an HCDR3 sequence set forth in one of SEQ ID NOs: 3, 8, and 13. In some embodiments, the anti-ASPH antibody or antigen binding fragment thereof comprises a variable light chain sequence as set forth in any one of SEQ ID NOs: 17, 19, and 22 and/or a variable heavy chain sequence as set forth in any one of SEQ ID NOs: 16, 18, 20 and 21.

In particular embodiments, an ASPH-binding domain comprises an alternative (e.g., non-immunoglobulin) scaffold. In some embodiments, an antigen-binding domain that specifically binds to human ASPH comprises or consists of a Fibronectin type III domain, a Tn3 protein, a designed ankyrin repeat protein (DARpin®), an affibody, an ANTICALIN®, an anti-ASPH aptamer, an EGF-like domain, a human Notch receptor derivative or a human Notch ligand derivative.

Protein scaffolds of the disclosure may provide enhanced biophysical properties, such as stability under reducing conditions and solubility at high concentrations; they may be expressed and folded in prokaryotic systems, such as E. coli, in eukaryotic systems, such as yeast, and in in vitro transcription/translation systems, such as the rabbit reticulocyte lysate system.

In some embodiments, the protein scaffolds of the disclosure offer advantages over conventional therapeutics, such as ability to administer locally, orally, or cross the blood-brain barrier, ability to express in E. coli allowing for increased expression of protein as a function of resources versus mammalian cell expression, ability to be engineered into bispecific or tandem molecules that bind to multiple targets or multiple epitopes of the same target, ability to be conjugated to drugs, polymers, and probes, ability to be formulated to high concentrations, and the ability of such molecules to effectively penetrate diseased (e.g., cancerous) tissues and tumors.

In some embodiments, ASPH-binding domains may comprise protein scaffolds derived from a fibronectin type III (FN3) repeat protein. In some embodiments, the protein scaffold comprises a consensus sequence of multiple FN3 domains from human Tenascin-C (hereinafter “Tenascin”). In one embodiment, the protein scaffold is a consensus sequence of 15 FN3 domains.

In some embodiments, ASPH-binding domains may comprise an antibody mimetic. The term “antibody mimetic” can describe an organic compound that specifically binds a target sequence and has a structure distinct from a naturally-occurring antibody. Antibody mimetics may comprise a protein, a nucleic acid, or a small molecule. The target sequence to which an antibody mimetic specifically binds may be ASPH. Antibody mimetics may provide superior properties over antibodies including, but not limited to, superior solubility, tissue penetration, stability towards heat and enzymes (e.g., resistance to enzymatic degradation), and lower production costs. Exemplary antibody mimetics include, but are not limited to, an affibody, an afflilin, an affimer, an affitin, an alphabody, an anticalin, and an avimer (also known as avidity multimer), a DARpin® (Designed Ankyrin Repeat Protein), a Fynomer, a Kunitz domain peptide, and a monobody.

Affibody molecules of the disclosure comprise a protein scaffold comprising or consisting of one or more alpha helix without any disulfide bridges. In some embodiments, affibody molecules of the disclosure comprise or consist of three alpha helices. For example, an affibody molecule of the disclosure may comprise an immunoglobulin binding domain. An affibody molecule of the disclosure may comprise the Z domain of protein A.

Affilin molecules of the disclosure may comprise a protein scaffold produced by modification of exposed amino acids of, for example, either gamma-B crystallin or ubiquitin. Affilin molecules functionally mimic an antibody's affinity to antigen, but do not structurally mimic an antibody. In any protein scaffold used to make an affilin, those amino acids that are accessible to solvent or possible binding partners in a properly-folded protein molecule are considered exposed amino acids. Any one or more of these exposed amino acids may be modified to specifically bind to a target sequence or antigen (e.g. ASPH).

Affimer molecules of the disclosure may comprise a protein scaffold comprising a highly stable protein engineered to display peptide loops that provide a high affinity binding site for a specific target sequence. Exemplary affimer molecules of the disclosure comprise a protein scaffold based upon a cystatin protein or tertiary structure thereof. Exemplary affimer molecules of the disclosure may share a common tertiary structure of comprising an alpha-helix lying on top of an anti-parallel beta-sheet.

Affitin molecules of the disclosure may comprise an artificial protein scaffold, the structure of which may be derived, for example, from a DNA binding protein (e.g., the DNA binding protein Sac7d). Affitins of the disclosure selectively bind a target sequence, which may be the entirety or part of an antigen (e.g., ASPH). Exemplary affitins of the disclosure are manufactured by randomizing one or more amino acid sequences on the binding surface of a DNA binding protein and subjecting the resultant protein to ribosome display and selection. Target sequences of affitins of the disclosure may be found, for example, in the genome or on the surface of a peptide, protein, virus, or bacteria. In certain embodiments of the disclosure, an affitin molecule may be used as a specific inhibitor of an enzyme. Affitin molecules of the disclosure may include heat-resistant proteins or derivatives thereof.

Alphabody molecules of the disclosure may also be referred to as Cell-Penetrating Alphabodies (CPAB). Alphabody molecules of the disclosure may comprise small proteins (typically of less than 10 kDa) that bind to a variety of target sequences (including antigens). Alphabody molecules are capable of reaching and binding to intracellular target sequences. Structurally, alphabody molecules of the disclosure comprise an artificial sequence forming single chain alpha helix (similar to naturally occurring coiled-coil structures). Alphabody molecules of the disclosure may comprise a protein scaffold comprising one or more amino acids that are modified to specifically bind target proteins. Regardless of the binding specificity of the molecule, alphabody molecules of the disclosure maintain correct folding and thermostability.

Anticalin molecules of the disclosure may comprise artificial proteins that bind to target sequences or sites in either proteins or small molecules. Anticalin molecules of the disclosure may comprise an artificial protein derived from a human lipocalin. Anticalin molecules of the disclosure may be used in place of, for example, monoclonal antibodies or fragments thereof. Anticalin molecules may demonstrate superior tissue penetration and thermostability than monoclonal antibodies or fragments thereof. Exemplary anticalin molecules of the disclosure may comprise about 180 amino acids, having a mass of approximately 20 kDa.

Structurally, anticalin molecules of the disclosure may comprise a barrel structure comprising antiparallel beta-strands pairwise connected by loops and an attached alpha helix. In some embodiments, anticalin molecules of the disclosure comprise a barrel structure comprising eight antiparallel beta-strands pairwise connected by loops and an attached alpha helix.

Avimer molecules of the disclosure may comprise an artificial protein that specifically binds to a target sequence (which may also be an antigen). Avimers of the disclosure may recognize multiple binding sites within the same target or within distinct targets. When an avimer of the disclosure recognize more than one target, the avimer mimics function of a bi-specific antibody. The artificial protein avimer may comprise two or more peptide sequences of approximately 30-35 amino acids each. These peptides may be connected via one or more linker peptides. Amino acid sequences of one or more of the peptides of the avimer may be derived from an A domain of a membrane receptor. Avimers have a rigid structure that may optionally comprise disulfide bonds and/or calcium. Avimers of the disclosure may demonstrate greater heat stability compared to an antibody.

DARPins (Designed Ankyrin Repeat Proteins) of the disclosure may comprise genetically-engineered, recombinant, or chimeric proteins having high specificity and high affinity for a target sequence. In certain embodiments, DARPins of the disclosure are derived from ankyrin proteins and, optionally, comprise at least three repeat motifs (also referred to as repetitive structural units) of the ankyrin protein. Ankyrin proteins mediate high-affinity protein-protein interactions. DARPins of the disclosure comprise a large target interaction surface.

Fynomers of the disclosure may comprise small binding proteins (about 7 kDa) derived from the human Fyn SH3 domain and engineered to bind to target sequences and molecules with equal affinity and equal specificity as an antibody.

Kunitz domain peptides of the disclosure may comprise a protein scaffold comprising a Kunitz domain. Kunitz domains comprise an active site for inhibiting protease activity. Structurally, Kunitz domains of the disclosure comprise a disulfide-rich alpha+beta fold. This structure is exemplified by the bovine pancreatic trypsin inhibitor. Kunitz domain peptides recognize specific protein structures and serve as competitive protease inhibitors. Kunitz domains of the disclosure may comprise Ecallantide (derived from a human lipoprotein-associated coagulation inhibitor (LACI)).

Monobodies of the disclosure are small proteins (comprising about 94 amino acids and having a mass of about 10 kDa) comparable in size to a single chain antibody. These genetically engineered proteins specifically bind target sequences including antigens. Monobodies of the disclosure may specifically target one or more distinct proteins or target sequences. In some embodiments, monobodies of the disclosure comprise a protein scaffold mimicking the structure of human fibronectin, and more preferably, mimicking the structure of the tenth extracellular type III domain of fibronectin. The tenth extracellular type III domain of fibronectin, as well as a monobody mimetic thereof, contains seven beta sheets forming a barrel and three exposed loops on each side corresponding to the three complementarity determining regions (CDRs) of an antibody. In contrast to the structure of the variable domain of an antibody, a monobody lacks any binding site for metal ions as well as a central disulfide bond. Multispecific monobodies may be optimized by modifying the loops BC and FG. Monobodies of the disclosure may comprise an adnectin.

In some embodiments, the extracellular domain of the CAR is a bi-specific binding domain comprising a first binding site specific for ASPH and a second binding site specific for a biomarker of interest, which may be a tumor-associated antigen. Exemplary biomarkers of interest include cell-surface markers associated with various cancers including, but not limited to, BCMA (B-cell maturation antigen), CEA, CD5, CD19, CD20, CD22, CD33, CD38, CD70, CD123, CD133, CD138, EGFR-VII, EpCam, EphA2, Pan-ErbB, GAP, GD2, GPC3, Her2, IL-13Rα2, immunoglobulin kappa light chain, Lewis-Y, Mesothelin, Muc1, mucin 16, PSCA, PSMA, ROR1, and VEGFR2.

Antigen binding sequences for such biomarkers and CAR constructs comprising such antigen binding sequence are known in the art. For example, CEA-specific CARs (Katz et al., Clin Cancer Res (2015) 21(14):3149-3159), CD19-specific CARs (Axicabtagene ciloleucel (Yescarta®) and Tisagenlecleucel (Kymriah®), EGFRvIII-specific CARs (Morgan et al., Hum Gene Ther (2012) 23(10):1043-1053), EGF-R-specific CARs (Kobold et al., J Natl Cancer Inst (2014) 107(1):364), ErbB2-specific CARs (Wilkie et al., J Clin Immunol (2012) 32(5):1059-1070), GD2-specific CARs (Louis et al., Blood (2011) 118(23):6050-6056; Caruana et al., Nat Med (2015) 21(5):524-529), HER2-specific CARs (Ahmed et al., J Clin Oncol (2015) 33(15)1688-1696; Nakazawa et al., Mol Ther (2011) 19(12):2133-2143; Ahmed et al., Mol Ther (2009) 17(10):1779-1787; Luo et al., Cell Res (2016) 26(7):850-853; Morgan et al., Mol Ther (2010) 18(4):843-851; Grada et al., Mol Ther Nucleic Acids (2013) 9(2):32), IL13Rα2-specific CARs (Brown et al., Clin Cacner Res (2015) 21(18):4062-4072), MSLN-specific CARs (Moon et al, Clin Cancer Res (2011) 17(14):4719-30), and VEGF-R-specific CARs (Chinnasamy et al., Cancer Res (2016) 22(2):436-447).

In some embodiments, the binding domain of the CAR is specific for a tag molecule. In some embodiments, the binding domain of the CAR recognizes a tag molecule fused or conjugated to an anti-ASPH antibody or antigen-binding fragment thereof. In such embodiments, the antigen-specificity of the CAR is dependent on the antigen-specificity of the labeled antibody, such that a single CAR construct can be used to target multiple different antigens by substituting one antibody for another (See e.g., U.S. Pat. Nos. 9,233,125 and 9,624,279; US Patent Application Publication Nos. 20150238631 and 20180104354). Tag molecules suitable for use according to this embodiment include detectable tags, such as FITC or fluorescent proteins including GFP. For example, an anti-ASPH antibody or binding fragments thereof can be labeled with a FITC molecule and can be used in combination with a FITC-specific CAR. Anti-ASPH antibody or binding fragments thereof are described herein and are known in the art (See e.g., Wittrup et al.: U.S. Pat. No. 7,413,737; Yeung et al., 2007; both incorporated by reference herein in their entireties). Further FITC-specific antigen binding constructs are known in the art (Vaughan et al., Nat. Biotechnol. (1996) 14, 309-314) and can be used to construct FITC-specific CARs according to the description provided herein.

### Linkers

In certain embodiments, anti-ASPH CARs comprise linker residues between the various domains, e.g., added for appropriate spacing and conformation of the molecule. In particular embodiments, CARs comprise one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

Illustrative examples of linkers include glycine polymers (G)ₙ; glycine-serine polymers (G₁₋₅S₁₋₅)ₙ, where n is an integer of at least one, two, three, four, or five; glycine-alanine polymers; alanine-serine polymers; and other flexible linkers known in the art. Glycine and glycine-serine polymers are relatively unstructured, and therefore may be able to serve as a neutral tether between domains of fusion proteins such as the CARs described herein. Glycine accesses significantly more phi-psi space than even alanine, and is much less restricted than residues with longer side chains (see Scheraga, Rev. Computational Chem. 11173-142 (1992)). The ordinarily skilled artisan will recognize that design of a CAR in particular embodiments can include linkers that are all or partially flexible, such that the linker can include a flexible linker as well as one or more portions that confer less flexible structure to provide for a desired CAR structure.

Other exemplary linkers include, but are not limited to the following amino acid sequences: GGG; DGGGS (SEQ ID NO: 52); TGEKP (SEQ ID NO: 53) (see, e.g., Liu et al., PNAS 5525-5530 (1997)); GGRR (SEQ ID NO: 54) (Pomerantz et al. 1995, supra); (GGGGS)ₙ wherein=1, 2, 3, 4 or 5 (SEQ ID NO: 55) (Kim et al., PNAS 93, 1156-1160 (1996.); EGKSSGSGSESKVD (SEQ ID NO: 56) (Chaudhary et al., 1990, Proc. Natl. Acad. Sci. U.S.A. 87:1066-1070); KESGSVSSEQLAQFRSLD (SEQ ID NO: 57) (Bird et al., 1988, Science 242:423-426), GGRRGGGS (SEQ ID NO: 58); LRQRDGERP (SEQ ID NO: 59); LRQKDGGGSERP (SEQ ID NO: 60); LRQKDGGGSGGGSERP (SEQ ID NO: 61); or GSTSGSGKPGSGEGSTKG (SEQ ID NO: 62) (Cooper et al., Blood, 101(4): 1637-1644 (2003)). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods.

### Signal Peptide

In some embodiments, the extracellular domain of an anti-ASPH CAR comprises a signal peptide. In some embodiments, the signal peptide comprises a sequence encoding a human CD2, CD3δ, CD3ε, CD3γ, CD3ζ, CD4, CD8α, CD19, CD28, CD37, CD45, 4-1BB, GM-CSFR, IL-2, CD33, Human IgKVIII, Human IgG2 H, Chymotrypsinogen, trypsinogen-2, HSA, Insulin or tPA signal peptide.

### Spacer Domain

In particular embodiments, the extracellular domain of an anti-ASPH CAR comprises one or more “spacer domains,” which refers to the region that moves the antigen binding domain away from the effector cell surface to enable proper cell/cell contact, antigen binding and activation (Patel et al., Gene Therapy, 1999; 6: 412-419). In some embodiments, a CAR comprises a spacer domain between an antigen-binding domain and a transmembrane (TM) domain. The spacer domain may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region. In one embodiment, the spacer domain comprises the CH2 and CH3 of IgG1, IgG4, or IgD.

### Hinge Domain

In some embodiments, the extracellular domain of an anti-ASPH CAR comprises one or more “hinge domains,” which play a role in positioning the antigen binding domain away from the effector cell surface to enable proper cell/cell contact, antigen binding and activation. An anti-ASPH CAR generally comprises one or more hinge domains between the antigen-binding domain and the transmembrane (TM) domain. The hinge domain may be derived either from a natural, synthetic, semi-synthetic, or recombinant source. The hinge domain can include the amino acid sequence of a naturally occurring immunoglobulin hinge region or an altered immunoglobulin hinge region. Illustrative hinge domains suitable for use in the CARs described herein include the hinge region derived from CD8α and CD28, which may be wild-type hinge regions from these molecules or may be altered. In some embodiments, the hinge region comprises a sequence derived from a human CD8α, IgG4, and/or CD4 sequence.

### Transmembrane Domain

The “transmembrane (TM) domain” or “transmembrane (TM) region” is the portion of an anti-ASPH CAR that fuses the extracellular binding portion and intracellular signaling domain and anchors the CAR to the plasma membrane of the immune effector cell. The TM domain may be derived either from a natural, synthetic, semi-synthetic, or recombinant source. The TM domain may be derived from (i.e., comprise at least the transmembrane region(s) of) the alpha or beta chain of the T-cell receptor, CD2, CD3δ, CD3ε, CD3γ, CD3ζ, CD4, CD5, CD8α, CD9, CD16, CD19, CD22, CD27, CD28, CD33, CD37, CD45, CD64, CD80, CD86, CD134, CD137, CD152, CD154, 4-1BB, GM-CSFR or PD1. Persons of skill are aware of numerous transmembrane regions and the structural elements (such as lipophilic amino acid regions) that produce transmembrane domains in numerous membrane proteins and therefore can substitute any convenient sequence.

In an embodiment of the invention, the transmembrane domain comprises a CD8α transmembrane domain or a CD28 transmembrane domain. In some embodiments, the CD8 and CD28 are derived from the human CD8α or CD28 sequences. The CD8α or CD28 may comprise less than the whole CD8α or CD28, respectively. In this regard, in some embodiments, the CAR comprises a CD8α transmembrane domain comprising, consisting of, or consisting essentially of SEQ ID NO: 29 or 31 and/or a CD28 transmembrane domain comprising, consisting of, or consisting essentially of SEQ ID NO: 32 or 34.

### Intracellular Domain

In particular embodiments, anti-ASPH CARs comprise an intracellular domain. An “intracellular domain,” refers to the part of a CAR that participates in transducing the message of effective anti-ASPH CAR binding to a human ASPH polypeptide into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. The intracellular domain may comprise one or more signaling domains.

The term “effector function” refers to a specialized function of an immune effector cell. Effector function of the T cell, for example, may be cytolytic activity or help or activity including the secretion of a cytokine. Thus, the term “signaling domain” refers to the portion of a protein which transduces the effector function signal and that directs the cell to perform a specialized function. While usually the entire signaling domain can be employed, in many cases it is not necessary to use the entire domain. To the extent that a truncated portion of a signaling domain is used, such truncated portion may be used in place of the entire domain as long as it transduces the effector function signal. The term signaling domain is meant to include any truncated portion of the signaling domain sufficient to transducing effector function signal. In some embodiments, the signaling domain of a CAR comprises one or more of an intracellular signaling portion of human CD3 zeta, CD28, CD137, TCR zeta, FcR gamma, FcR beta, CD3 gamma, CD3 delta or CD3 epsilon.

In some cases, signals generated through the TCR alone may be insufficient for full activation of the effector cell (e.g., T cell) and that a secondary or co-stimulatory signal may also be required. Thus, effector cell (e.g., T cell) activation can be said to be mediated by two distinct classes of intracellular signaling domains: signaling domains that initiate antigen-dependent primary activation through the TCR (e.g., a TCR/CD3 complex) and co-stimulatory domains that act in an antigen-independent manner to provide a secondary or co-stimulatory signal. In some embodiments, a CAR comprises an intracellular domain that comprises one or more “co-stimulatory domains” and a “signaling domain.”

Signaling domains regulate primary activation of the TCR complex either in a stimulatory way, or in an inhibitory way. Signaling domains that act in a stimulatory manner may contain signaling motifs which are known as immunoreceptor tyrosine-based activation motifs or ITAMs. Illustrative examples of ITAM containing signaling domains that are useful in particular embodiments include those derived from FcRγ, FcRβ, CD3γ, CD3δ, CD3ε, CD3ζ, CD22, CD79a, CD79b, and CD66d. In particular embodiments, an anti-ASPH CAR comprises a CD3ζ signaling domain and one or more co-stimulatory domains. The intracellular signaling and co-stimulatory domains may be linked in any order in tandem to the carboxyl terminus of the transmembrane domain. In some embodiments, the intracellular domain comprises a CD3ζ signaling domain amino acid sequence comprising, consisting of, or consisting essentially of, SEQ ID NO: 37.

In particular embodiments, CARs comprise one or more co-stimulatory domains to enhance the efficacy and expansion of T cells expressing CAR receptors. As used herein, the term “co-stimulatory domain”, refers to an intracellular domain of a co-stimulatory molecule. Co-stimulatory molecules are cell surface molecules other than antigen receptors or Fc receptors that provide a second signal required for efficient activation and function of T lymphocytes upon binding to antigen. Illustrative examples of such co-stimulatory molecules include TLR1, TLR2, TLR3, TLR4, TLR5, TLR6, TLR7, TLR8, TLR9, TLR10, CARD11, CD2, CD7, CD27, CD28, CD30, CD40, CD40LG (CD40L), CD54 (ICAM), CD83, CD134 (OX40), CD137 (4-1BB), CD278 (ICOS), DAP10, DAP-12, ITGB2 (LFA-1), LAT, MyD88, NKD2C (KLRC2), SLP76, TNFRS18 (GITR), TNFRSF14 (HVEM), TRIM, and ZAP70. In one embodiment, a CAR comprises one or more co-stimulatory domains selected from CD28 and CD137, and a CD3ζ signaling domain.

In some embodiments, the intracellular domain comprises one or more co-stimulatory domains selected from CD28 and 4-1BB. Signaling via CD28 is required for IL2 production and proliferation, but does not play a primary role in sustaining T cell function and activity. 4-1BB (a tumor necrosis factor-receptor family member expressed following CD28 activation) and OX-40 are involved in driving long-term survival of T cells, and accumulation of T cells. The ligands for these receptors typically are expressed on professional antigen presenting cells such as dendritic cells and activated macrophages, but not on tumor cells.

In some embodiments, the intracellular domain of a CAR comprises a CD28 co-stimulatory domain and a CD3ζ signaling domain. In some embodiments, the intracellular domain of a CAR comprises a 4-1BB co-stimulatory domain and a CD3ζ signaling domain. In some embodiments, the CD28, 4-1BB, and CD3ζ domains are human. In some embodiments, expressing a CAR that incorporates CD28 and/or 4-1BB signaling domains in CD4⁺ T cells enhances the activity and anti-tumor potency of those cells compared to those expressing a CAR that contains only the CD3ζ signaling domain. In some embodiments, the anti-ASPH CARs contain both CD28 and 4-1BB co-stimulatory domains.

In some embodiments, the intracellular domain comprises a CD28 amino acid sequence comprising, consisting of, or consisting essentially of, SEQ ID NO: 35. In some embodiments, the intracellular domain comprises a 4-1BB amino acid sequence comprising, consisting of, or consisting essentially of, SEQ ID NO: 36. In some embodiments, the intracellular domain comprises a CD28 co-stimulatory domain and a CD3ζ signaling domain, wherein the CD28 amino acid sequence comprises, consists of, or consists essentially of, SEQ ID NO: 35 and wherein the CD3 amino acid sequence comprises, consists of, or consists essentially of, SEQ ID NO: 37. In some embodiments, the intracellular domain comprises a 4-1BB co-stimulatory domain and a CD3ζ signaling domain, wherein the 4-1BB amino acid sequence comprises, consists of, or consists essentially of, SEQ ID NO: 36, and wherein the CD3 amino acid sequence comprises, consists of, or consists essentially of, SEQ ID NO: 37.

### Exemplary CAR Constructs

In some embodiments, the anti-ASPH CAR constructs provided herein comprise an anti-ASPH binding domain, a hinge domain, a transmembrane domain, and an intracellular domain. In some embodiments, the intracellular domain comprises a signaling domain. In some embodiments, the intracellular domain comprises a signaling domain and a costimulatory domain. In some embodiments, the anti-ASPH CAR constructs provided herein comprise, in amino-terminal to carboxyl-terminal order, an scFv that specifically binds to human ASPH, a spacer, a transmembrane domain, a costimulatory domain and a signaling domain.

In some embodiments, the anti-ASPH binding domain comprises an immunoglobulin VH chain, comprising HCDR1, HCDR2, and HCDR3, and an immunoglobulin VL chain comprising LCDR1, LCDR2, and LCDR3, wherein the HCDR1 comprises an amino acid sequence of SEQ ID NO: 1; the HCDR2 comprises an amino acid sequence of SEQ ID NO: 2; and the HCDR3 comprises an amino acid sequence of SEQ ID NO: 3; and wherein the LCDR1 comprises an amino acid sequence of SEQ ID NO: 4; the LCDR2 comprises an amino acid sequence of SEQ ID NO: 5; the LCDR3 comprises an amino acid sequence of SEQ ID NO: 6.

In some embodiments, the anti-ASPH binding domain comprises an immunoglobulin VH chain, comprising HCDR1, HCDR2, and HCDR3, and an immunoglobulin VL chain comprising LCDR1, LCDR2, and LCDR3, wherein the HCDR1 comprises an amino acid sequence of SEQ ID NO: 7; the HCDR2 comprises an amino acid sequence of SEQ ID NO: 2; and the HCDR3 comprises an amino acid sequence of SEQ ID NO: 8; and wherein the LCDR1 comprises an amino acid sequence of SEQ ID NO: 4; the LCDR2 comprises an amino acid sequence of SEQ ID NO: 9; the LCDR3 comprises an amino acid sequence of SEQ ID NO: 10.

In some embodiments, the anti-ASPH binding domain comprises an immunoglobulin VH chain, comprising HCDR1, HCDR2, and HCDR3, and an immunoglobulin VL chain comprising LCDR1, LCDR2, and LCDR3, wherein the HCDR1 comprises an amino acid sequence of SEQ ID NO: 11; the HCDR2 comprises an amino acid sequence of SEQ ID NO: 12; and the HCDR3 comprises an amino acid sequence of SEQ ID NO: 13; and wherein the LCDR1 comprises an amino acid sequence of SEQ ID NO: 4; the LCDR2 comprises an amino acid sequence of SEQ ID NO: 5; the LCDR3 comprises an amino acid sequence of SEQ ID NO: 6.

In some embodiments, the anti-ASPH binding domain comprises an immunoglobulin VH chain, comprising HCDR1, HCDR2, and HCDR3, and an immunoglobulin VL chain comprising LCDR1, LCDR2, and LCDR3, wherein the HCDR1 comprises an amino acid sequence of SEQ ID NO: 1; the HCDR2 comprises an amino acid sequence of SEQ ID NO: 14; and the HCDR3 comprises an amino acid sequence of SEQ ID NO: 15; and wherein the LCDR1 comprises an amino acid sequence of SEQ ID NO: 4; the LCDR2 comprises an amino acid sequence of SEQ ID NO: 5; the LCDR3 comprises an amino acid sequence of SEQ ID NO: 6.

Exemplary CDR sequences of the anti-ASPH binding domains described herein are provided below in Table 1.

TABLE 1
Exemplary anti-ASPH binding domain CDR sequences

| Construct | CDR | Amino Acid Sequence | SEQ ID: |
|---|---|---|---|
| 622 | HCDR1 | SNSAAWN | 1 |
| | HCDR2 | RTYYRSKWYNDYAVSVKS | 2 |
| | HCDR3 | TGYSSSWVVNFDY | 3 |
| | LCDR1 | SGSSSNIGSNYVY | 4 |
| | LCDR2 | KLLIYKNNQRPS | 5 |
| | LCDR3 | AAWDDSLRGYV | 6 |
| 623 | HCDR1 | SDSAAWN | 7 |
| | HCDR2 | RTYYRSKWYNDYAVSVKS | 2 |
| | HCDR3 | AQNNIAVAGFDY | 8 |
| | LCDR1 | SGSSSNIGSNYVY | 4 |
| | LCDR2 | TLLIYRNNQRPS | 9 |
| | LCDR3 | AAWDDSLSGLYV | 10 |
| LLm13 | HCDR1 | ADRVAWN | 11 |
| | HCDR2 | RIFYRSKWMVDYAVSVKS | 12 |
| | HCDR3 | ATTRGYFDL | 13 |
| | LCDR1 | SGSSSNIGSNYVY | 4 |
| | LCDR2 | KLLIYKNNQRPS | 5 |
| | LCDR3 | AAWDDSLRGYV | 6 |
| C4m18 | HCDR1 | SNSAAWN | 1 |
| | HCDR2 | RTYYRSKWYNGYAVSVRG | 14 |
| | HCDR3 | TGYSSSWVVNSNY | 15 |
| | LCDR1 | SGSSSNIGSNYVY | 4 |
| | LCDR2 | KLLIYKNNQRPS | 5 |
| | LCDR3 | AAWDDSLRGYV | 6 |

In some embodiments, the anti-ASPH binding domain comprises an immunoglobulin VH chain comprising an amino acid sequence that is at least 90%, 95%, 96%, 97%, 98%, or 99% identical to SEQ ID NO: 16 and an immunoglobulin VL chain comprising an amino acid sequence that is at least 90%, 95%, 96%, 97%, 98%, or 99% identical to SEQ ID NO: 17. In some embodiments, the anti-ASPH binding domain comprises an immunoglobulin VH chain comprising an amino acid sequence that is 100% identical to SEQ ID NO: 16 and an immunoglobulin VL chain comprising an amino acid sequence that is 100% identical to SEQ ID NO: 17. In some embodiments, the anti-ASPH binding domain comprises an immunoglobulin VH chain consisting of, or consisting essentially of, the amino acid sequence of SEQ ID NO: 16 and an immunoglobulin VL chain consisting of, or consisting essentially of, the amino acid sequence of SEQ ID NO: 17.

In some embodiments, the anti-ASPH binding domain comprises an immunoglobulin VH chain comprising an amino acid sequence that is at least 90%, 95%, 96%, 97%, 98%, or 99% identical to SEQ ID NO: 18 and an immunoglobulin VL chain comprising an amino acid sequence that is at least 90%, 95%, 96%, 97%, 98%, or 99% identical to SEQ ID NO: 19. In some embodiments, the anti-ASPH binding domain comprises an immunoglobulin VH chain comprising an amino acid sequence that is 100% identical to SEQ ID NO: 18 and an immunoglobulin VL chain comprising an amino acid sequence that is 100% identical to SEQ ID NO: 19. In some embodiments, the anti-ASPH binding domain comprises an immunoglobulin VH chain consisting of, or consisting essentially of, the amino acid sequence of SEQ ID NO: 18 and an immunoglobulin VL chain consisting of, or consisting essentially of, the amino acid sequence of SEQ ID NO: 19.

In some embodiments, the anti-ASPH binding domain comprises an immunoglobulin VH chain comprising an amino acid sequence that is at least 90%, 95%, 96%, 97%, 98%, or 99% identical to SEQ ID NO: 20 and an immunoglobulin VL chain comprising an amino acid sequence that is at least 90%, 95%, 96%, 97%, 98%, or 99% identical to SEQ ID NO: 17. In some embodiments, the anti-ASPH binding domain comprises an immunoglobulin VH chain comprising an amino acid sequence that is 100% identical to SEQ ID NO: 20 and an immunoglobulin VL chain comprising an amino acid sequence that is 100% identical to SEQ ID NO: 17. In some embodiments, the anti-ASPH binding domain comprises an immunoglobulin VH chain consisting of, or consisting essentially of, the amino acid sequence of SEQ ID NO: 20 and an immunoglobulin VL chain consisting of, or consisting essentially of, the amino acid sequence of SEQ ID NO: 17.

In some embodiments, the anti-ASPH binding domain comprises an immunoglobulin VH chain comprising an amino acid sequence that is at least 90%, 95%, 96%, 97%, 98%, or 99% identical to SEQ ID NO: 21 and an immunoglobulin VL chain comprising an amino acid sequence that is at least 90%, 95%, 96%, 97%, 98%, or 99% identical to SEQ ID NO: 22. In some embodiments, the anti-ASPH binding domain comprises an immunoglobulin VH chain comprising an amino acid sequence that is 100% identical to SEQ ID NO: 21 and an immunoglobulin VL chain comprising an amino acid sequence that is 100% identical to SEQ ID NO: 22. In some embodiments, the anti-ASPH binding domain comprises an immunoglobulin VH chain consisting of, or consisting essentially of, the amino acid sequence of SEQ ID NO: 21 and an immunoglobulin VL chain consisting of, or consisting essentially of, the amino acid sequence of SEQ ID NO: 22.

Exemplary VH and VL sequences of the anti-ASPH binding domains described herein are provided below in Table 2.

TABLE 2
Exemplary anti-ASPH binding domain VH and VL sequences

| | Variable | | SEQ |
|---|---|---|---|
| Construct | domain | Amino Acid Sequence | ID: |
| 622 | VH | QVQLQQSGPGLVKPSQTLSLTCAISGDSVSSNSAAWNWIRQSPSRGLEW | 16 |
| | | LGRTYYRSKWYNDYAVSVKSRITINPDTSKNQFSLQLNSVTPEDTAVYY | |
| | | CARTGYSSSWVVNFDYWGQGTLVTVSSGSASAPTGIL | |
| | VL | QPVLTQSPSASGTPGQRVTISCSGSSSNIGSNYVYWYQQLPGTAPKLLI | 17 |
| | | YKNNQRPSGVPDRFSGSKSGTAASLAISGLQSEDEADYYCAAWDDSLRG | |
| | | YVFGTGTKLTVLSG | |
| 623 | VH | QVQLQQSGPGLVKPSQTLSLTCAISGDSVSSDSAAWNWIRQSPSRGLEW | 18 |
| | | LGRTYYRSKWYNDYAVSVKSRISINPDTSKNQFSLQLNSVTPEDTAVYY | |
| | | CARAQNNIAVAGFDYWGLGTLVTVSSGIL | |
| | VL | QPVLTQSPSASGTPGQRVTISCSGSSSNIGSNYVYWYQQLPGTAPTLLI | 19 |
| | | YRNNQRPSGVPDRFSGSKSGTSASLAISGLRSEDEAEYYCAAWDDSLSG | |
| | | LYVFGTGTKVTVLSGIL | |
| LLm13 | VH | QVQLQQSGAGLVKPSQTLSLTCTISGDSVSADRVAWNWIRQSPLRGLEW | 20 |
| | | LGRIFYRSKWMVDYAVSVKSRISINPDTSKNQFSLQLNSVTPEDTAMYY | |
| | | CARATTRGYFDLWGRGTLVTVSSGIL | |
| | VL | QPVLTQSPSASGTPGQRVTISCSGSSSNIGSNYVYWYQQLPGTAPKLLI | 17 |
| | | YKNNQRPSGVPDRFSGSKSGTAASLAISGLQSEDEADYYCAAWDDSLRG | |
| | | YVFGTGTKLTVLSGIL | |
| C4m18 | VH | QVQLQQSGPGLVKPSPTLSLTCAISGDSVSSNSAAWNWVRQSLSRGLEW | 21 |
| | | LGRTYYRSKWYNGYAVSVRGRITTNADTSRNQFSLQLNSVTPEDTAVYY | |
| | | CARTGYSSSWVVNSNYWGQGTLVTVSSGSASAPTGIL | |
| | VL | QPALTQSPSASGTPGQRVTISCSGSSSNIGSNYVYWYQQLPGTAPKLLI | 22 |
| | | YKNNQRPSGVPGRFSGSKSGTAASLAISGLRSKDEADYYCAAWDDSLRG | |
| | | YVFGTGTKLTVL | |

In some embodiments, the anti-ASPH binding domain is an anti-ASPH scFv comprising an amino acid sequence that is at least 90%, 95%, 96%, 97%, 98%, or 99% identical to SEQ ID NO: 23. In some embodiments, the anti-ASPH binding domain is an anti-ASPH scFv comprising an amino acid sequence that is 100% identical to SEQ ID NO: 23. In some embodiments, the anti-ASPH binding domain is an anti-ASPH scFv consisting of, or consisting essentially of, the amino acid sequence of SEQ ID NO: 23.

In some embodiments, the anti-ASPH binding domain is an anti-ASPH scFv comprising an amino acid sequence that is at least 90%, 95%, 96%, 97%, 98%, or 99% identical to SEQ ID NO: 24. In some embodiments, the anti-ASPH binding domain is an anti-ASPH scFv comprising an amino acid sequence that is 100% identical to SEQ ID NO: 24. In some embodiments, the anti-ASPH binding domain is an anti-ASPH scFv consisting of, or consisting essentially of, the amino acid sequence of SEQ ID NO: 24.

In some embodiments, the anti-ASPH binding domain is an anti-ASPH scFv comprising an amino acid sequence that is at least 90%, 95%, 96%, 97%, 98%, or 99% identical to SEQ ID NO: 25. In some embodiments, the anti-ASPH binding domain is an anti-ASPH scFv comprising an amino acid sequence that is 100% identical to SEQ ID NO: 25. In some embodiments, the anti-ASPH binding domain is an anti-ASPH scFv consisting of, or consisting essentially of, the amino acid sequence of SEQ ID NO: 25.

In some embodiments, the anti-ASPH binding domain is an anti-ASPH scFv comprising an amino acid sequence that is at least 90%, 95%, 96%, 97%, 98%, or 99% identical to SEQ ID NO: 26. In some embodiments, the anti-ASPH binding domain is an anti-ASPH scFv comprising an amino acid sequence that is 100% identical to SEQ ID NO: 26. In some embodiments, the anti-ASPH binding domain is an anti-ASPH scFv consisting of, or consisting essentially of, the amino acid sequence of SEQ ID NO: 26.

Exemplary scFv sequences of the anti-ASPH binding domains described herein are provided below in Table 3.

TABLE 3
Exemplary anti-ASPH scFv sequences

| | | SEQ |
|---|---|---|
| Construct | scFv Amino Acid Sequence | ID: |
| 622 | QVQLQQSGPGLVKPSQTLSLTCAISGDSVSSNSAAWNWIRQSPSRGLEWLGRTYYRSK | 23 |
| | WYNDYAVSVKSRITINPDTSKNQFSLQLNSVTPEDTAVYYCARTGYSSSWVVNEDYWG | |
| | QGTLVTVSSGSASAPTGILGSGGGGSGGGGSGGGGSQPVLTQSPSASGTPGQRVTISC | |
| | SGSSSNIGSNYVYWYQQLPGTAPKLLIYKNNQRPSGVPDRFSGSKSGTAASLAISGLQ | |
| | SEDEADYYCAAWDDSLRGYVFGTGTKLTVLSG | |
| 623 | QVQLQQSGPGLVKPSQTLSLTCAISGDSVSSDSAAWNWIRQSPSRGLEWLGRTYYRSK | 24 |
| | WYNDYAVSVKSRISINPDTSKNQFSLQLNSVTPEDTAVYYCARAQNNIAVAGFDYWGL | |
| | GTLVTVSSGILGSGGGGSGGGGSGGGGSQPVLTQSPSASGTPGQRVTISCSGSSSNIG | |
| | SNYVYWYQQLPGTAPTLLIYRNNQRPSGVPDRFSGSKSGTSASLAISGLRSEDEAEYY | |
| | CAAWDDSLSGLYVFGTGTKVTVLSGIL | |
| LLm13 | QVQLQQSGAGLVKPSQTLSLTCTISGDSVSADRVAWNWIRQSPLRGLEWLGRIFYRSK | 25 |
| | WMVDYAVSVKSRISINPDTSKNQFSLQLNSVTPEDTAMYYCARATTRGYFDLWGRGTL | |
| | VTVSSGILGSGGGGSGGGGSGGGGSQPVLTQSPSASGTPGQRVTISCSGSSSNIGSNY | |
| | VYWYQQLPGTAPKLLIYKNNQRPSGVPDRFSGSKSGTAASLAISGLQSEDEADYYCAA | |
| | WDDSLRGYVFGTGTKLTVLSGIL | |
| C4m18 | QVQLQQSGPGLVKPSPTLSLTCAISGDSVSSNSAAWNWVRQSLSRGLEWLGRTYYRSK | 26 |
| | WYNGYAVSVRGRITTNADTSRNQFSLQLNSVTPEDTAVYYCARTGYSSSWVVNSNYWG | |
| | QGTLVTVSSGSASAPTGILGSGGGGSGGGGSGGGGSQPALTQSPSASGTPGQRVTISC | |
| | SGSSSNIGSNYVYWYQQLPGTAPKLLIYKNNQRPSGVPGRFSGSKSGTAASLAISGLR | |
| | SKDEADYYCAAWDDSLRGYVFGTGTKLTVL | |

In some embodiments, the anti-ASPH CAR constructs provided herein comprise an anti-ASPH binding domain, a CD8α hinge domain, a CD8α transmembrane domain, and an intracellular domain comprising a CD3ζ signaling domain. In some embodiments, anti-ASPH binding domain is an scFv domain comprising an amino acid sequence that is at least 90%, 95%, 96%, 97%, 98%, or 99% identical to one of SEQ ID NOs: 23-26. In some embodiments, anti-ASPH binding domain is an scFv domain comprising an amino acid sequence that is 100% identical to one of SEQ ID NOs: 23-26. In some embodiments, the anti-ASPH binding domain is an anti-ASPH scFv consisting of, or consisting essentially of, the amino acid sequence of one of SEQ ID NOs: 23-26.

In some embodiments, the anti-ASPH CAR constructs provided herein comprise an anti-ASPH binding domain, a CD8α hinge domain, a CD8α transmembrane domain, and an intracellular domain comprising a CD3ζ signaling domain and a 4-1BB costimulatory domain. In some embodiments, anti-ASPH binding domain is an scFv domain comprising an amino acid sequence that is at least 90%, 95%, 96%, 97%, 98%, or 99% identical to one of SEQ ID NOs: 23-26. In some embodiments, anti-ASPH binding domain is an scFv domain comprising an amino acid sequence that is 100% identical to one of SEQ ID NOs: 23-26. In some embodiments, the anti-ASPH binding domain is an anti-ASPH scFv consisting of, or consisting essentially of, the amino acid sequence of one of SEQ ID NOs: 23-26.

In some embodiments, the anti-ASPH CAR constructs provided herein comprise an anti-ASPH binding domain, a CD8α hinge domain, a CD8α transmembrane domain, and an intracellular domain comprising a CD3ζ signaling domain and a 4-1BB costimulatory domain. In some embodiments, the anti-ASPH CAR construct comprises an amino acid sequence that is at least 90%, 95%, 96%, 97%, 98%, or 99% identical to one of SEQ ID NOs: 40-47. In some embodiments, the anti-ASPH CAR construct comprises an amino acid sequence that is 100% identical to one of SEQ ID NOs: 40-47. In some embodiments, the anti-ASPH CAR construct consists of, or consists essentially of, the amino acid sequence of one of SEQ ID NOs: 40-47.

In some embodiments, the anti-ASPH CAR constructs provided herein comprise an anti-ASPH binding domain, a CD28 hinge domain, a CD28 transmembrane domain, and an intracellular domain comprising a CD3ζ signaling domain. In some embodiments, anti-ASPH binding domain is an scFv domain comprising an amino acid sequence that is at least 90%, 95%, 96%, 97%, 98%, or 99% identical to one of SEQ ID NOs: 23-26. In some embodiments, anti-ASPH binding domain is an scFv domain comprising an amino acid sequence that is 100% identical to one of SEQ ID NOs: 23-26. In some embodiments, the anti-ASPH binding domain is an anti-ASPH scFv consisting of, or consisting essentially of, the amino acid sequence of one of SEQ ID NOs: 23-26.

In some embodiments, the anti-ASPH CAR constructs provided herein comprise an anti-ASPH binding domain, a CD28 hinge domain, a CD28 transmembrane domain, and an intracellular domain comprising a CD3ζ signaling domain and a CD28 costimulatory domain. In some embodiments, anti-ASPH binding domain is an scFv domain comprising an amino acid sequence that is at least 90%, 95%, 96%, 97%, 98%, or 99% identical to one of SEQ ID NOs: 23-26. In some embodiments, anti-ASPH binding domain is an scFv domain comprising an amino acid sequence that is 100% identical to one of SEQ ID NOs: 23-26. In some embodiments, the anti-ASPH binding domain is an anti-ASPH scFv consisting of, or consisting essentially of, the amino acid sequence of one of SEQ ID NOs: 23-26.

In some embodiments, the anti-ASPH CAR constructs provided herein comprise an anti-ASPH binding domain, a CD28 hinge domain, a CD28 transmembrane domain, and an intracellular domain comprising a CD3ζ signaling domain and a CD28 costimulatory domain. In some embodiments, the anti-ASPH CAR construct comprises an amino acid sequence that is at least 90%, 95%, 96%, 97%, 98%, or 99% identical to one of SEQ ID NOs: 48-51. In some embodiments, the anti-ASPH CAR construct comprises an amino acid sequence that is 100% identical to one of SEQ ID NOs: 48-51. In some embodiments, the anti-ASPH CAR construct consists of, or consists essentially of, the amino acid sequence of one of SEQ ID NOs: 48-51.

TABLE 4
Exemplary CAR construct components

| Component | Amino Acid Sequence | SEQ ID: |
|---|---|---|
| GMCSFα SP | MLLLVTSLLLCELPHPAFLLIP | 27 |
| CD8α SP | MALPVTALLLPLALLLHAARP | 28 |
| CD8α hinge/TM | TTTPAPRPPTPAPTIASQPLSLRPEACRPAAGGAVHTRGLDFACDI | 29 |
| | YIWAPLAGTCGVLLLSLVITLYC | |
| CD8α hinge | TTTPAPRPPTPAPTIASQPLSLRPEACRPAAGGAVHTRGLDFACD | 30 |
| CD8α TM | IYIWAPLAGTCGVLLLSLVITLYC | 31 |
| CD28 hinge/TM/ | VKGKHLCPSPLFPGPSKPFWVLVVVGGVLACYSLLVTVAFIIFWVR | 32 |
| Costim | SKRSRLLHSDYMNMTPRRPGPTRKHYQPYAPPRDFAAYRS | |
| CD28 hinge | VKGKHLCPSPLFPGPSKP | 33 |
| CD28 TM | FWVLVVVGGVLACYSLLVTVAFIIFWV | 34 |
| CD28 Costim | RSKRSRLLHSDYMNMTPRRPGPTRKHYQPYAPPRDFAAYRS | 35 |
| 4-1BB costim | KRGRKKLLYIFKQPFMRPVQTTQEEDGCSCRFPEEEEGGCEL | 36 |
| domain | | |
| CD3ζ signaling | RVKFSRSADAPAYQQGQNQLYNELNLGRREEYDVLDKRRGRDPEMG | 37 |
| domain | GKPRRKNPQEGLYNELQKDKMAEAYSEIGMKGERRRGKGHDGLYQG | |
| | LSTATK | |
| GFP | SGMESDESGLPAMEIECRITGTLNGVEFELVGGGEGTPKQGRMTNK | 38 |
| | MKSTKGALTFSPYLLSHVMGYGFYHEGTYPSGYENPFLHAINNGGY | |
| | TNTRIEKYEDGGVLHVSFSYRYEAGRVIGDFKVVGTGFPEDSVIFT | |
| | DKIIRSNATVEHLHPMGDNVLVGSFARTFSLRDGGYYSFVVDSHMH | |
| | FKSAIHPSILQNGGPMFAFRRVEELHSNTELGIVEYQHAFKTPTAF | |
| | ARSRAQSSNSAVDGTAGPGSTGSR | |
| T2A | EGRGSLLTCGDVEENPGP | 39 |

Exemplary amino acid sequences for CAR constructs comprising a 4-1BB costimulatory sequence and a CD3 signaling sequence are provided below in Table 5. The general design of the constructs shown in Table 5 is: GMCSFα signal peptide (SP)—anti-ASPH scFv—CD8α hinge and TM—4-1BB costimulatory domain—CD3ζ signaling domain. BBz constructs comprise a GFP amino acid sequence linked via the T2A sequence to the C terminal end of the CAR construct. BBzΔGFP constructs do not comprise the C-terminal GFP sequence.

TABLE 5
Exemplary BBz CAR constructs

| Construct | Amino Acid Sequence | SEQ ID: |
|---|---|---|
| 622-BBZ | MLLLVTSLLLCELPHPAFLLIPQVQLQQSGPGLVKPSQTLSLTCAI | 40 |
| | SGDSVSSNSAAWNWIRQSPSRGLEWLGRTYYRSKWYNDYAVSVKSR | |
| | ITINPDTSKNQFSLQLNSVTPEDTAVYYCARTGYSSSWVVNFDYWG | |
| | QGTLVTVSSGSASAPTGILGSGGGGSGGGGSGGGGSQPVLTQSPSA | |
| | SGTPGQRVTISCSGSSSNIGSNYVYWYQQLPGTAPKLLIYKNNQRP | |
| | SGVPDRFSGSKSGTAASLAISGLQSEDEADYYCAAWDDSLRGYVFG | |
| | TGTKLTVLSGGGGSTTTPAPRPPTPAPTIASQPLSLRPEACRPAAG | |
| | GAVHTRGLDFACDIYIWAPLAGTCGVLLLSLVITLYCKRGRKKLLY | |
| | IFKQPFMRPVQTTQEEDGCSCRFPEEEEGGCELRVKFSRSADAPAY | |
| | QQGQNQLYNELNLGRREEYDVLDKRRGRDPEMGGKPRRKNPQEGLY | |
| | NELQKDKMAEAYSEIGMKGERRRGKGHDGLYQGLSTATKDTYDALH | |
| | MQALPPRKRRKRGSGAAAEGRGSLLTCGDVEENPGPSGMESDESGL | |
| | PAMEIECRITGTLNGVEFELVGGGEGTPKQGRMTNKMKSTKGALTF | |
| | SPYLLSHVMGYGFYHFGTYPSGYENPFLHAINNGGYTNTRIEKYED | |
| | GGVLHVSFSYRYEAGRVIGDFKVVGTGFPEDSVIFTDKIIRSNATV | |
| | EHLHPMGDNVLVGSFARTFSLRDGGYYSFVVDSHMHFKSAIHPSIL | |
| | QNGGPMFAFRRVEELHSNTELGIVEYQHAFKTPIAFARSRAQSSNS | |
| | AVDGTAGPGSTGSR | |
| 622-BBzΔGFP | MLLLVTSLLLCELPHPAFLLIPQVQLQQSGPGLVKPSQTLSLTCAI | 41 |
| | SGDSVSSNSAAWNWIRQSPSRGLEWLGRTYYRSKWYNDYAVSVKSR | |
| | ITINPDTSKNQFSLQLNSVTPEDTAVYYCARTGYSSSWVVNFDYWG | |
| | QGTLVTVSSGSASAPTGILGSGGGGSGGGGSGGGGSQPVLTQSPSA | |
| | SGTPGQRVTISCSGSSSNIGSNYVYWYQQLPGTAPKLLIYKNNQRP | |
| | SGVPDRFSGSKSGTAASLAISGLQSEDEADYYCAAWDDSLRGYVFG | |
| | TGTKLTVLSGGGGSTTTPAPRPPTPAPTIASQPLSLRPEACRPAAG | |
| | GAVHTRGLDFACDIYIWAPLAGTCGVLLLSLVITLYCKRGRKKLLY | |
| | IFKQPFMRPVQTTQEEDGCSCRFPEEEEGGCELRVKFSRSADAPAY | |
| | QQGQNQLYNELNLGRREEYDVLDKRRGRDPEMGGKPRRKNPQEGLY | |
| | NELQKDKMAEAYSEIGMKGERRRGKGHDGLYQGLSTATKDTYDALH | |
| | MQALPPR | |
| 623-BBz | MLLLVTSLLLCELPHPAFLLIPQVQLQQSGPGLVKPSQTLSLTCAI | 42 |
| | SGDSVSSDSAAWNWIRQSPSRGLEWLGRTYYRSKWYNDYAVSVKSR | |
| | ISINPDTSKNQFSLQLNSVTPEDTAVYYCARAQNNIAVAGFDYWGL | |
| | GTLVTVSSGILGSGGGGSGGGGSGGGGSQPVLTQSPSASGTPGQRV | |
| | TISCSGSSSNIGSNYVYWYQQLPGTAPTLLIYRNNQRPSGVPDRFS | |
| | GSKSGTSASLAISGLRSEDEAEYYCAAWDDSLSGLYVFGTGTKVTV | |
| | LSGILGGGGSTTTPAPRPPTPAPTIASQPLSLRPEACRPAAGGAVH | |
| | TRGLDFACDIYIWAPLAGTCGVLLLSLVITLYCKRGRKKLLYIFKQ | |
| | PFMRPVQTTQEEDGCSCRFPEEEEGGCELRVKFSRSADAPAYQQGQ | |
| | NQLYNELNLGRREEYDVLDKRRGRDPEMGGKPRRKNPQEGLYNELQ | |
| | KDKMAEAYSEIGMKGERRRGKGHDGLYQGLSTATKDTYDALHMQAL | |
| | PPRKRRKRGSGAAAEGRGSLLTCGDVEENPGPSGMESDESGLPAME | |
| | IECRITGTLNGVEFELVGGGEGTPKQGRMTNKMKSTKGALTFSPYL | |
| | LSHVMGYGFYHFGTYPSGYENPFLHAINNGGYTNTRIEKYEDGGVL | |
| | HVSFSYRYEAGRVIGDFKVVGTGFPEDSVIFTDKIIRSNATVEHLH | |
| | PMGDNVLVGSFARTFSLRDGGYYSFVVDSHMHFKSAIHPSILQNGG | |
| | PMFAFRRVEELHSNTELGIVEYQHAFKTPIAFARSRAQSSNSAVDG | |
| | TAGPGSTGSR | |
| 623-BBzΔGFP | MLLLVTSLLLCELPHPAFLLIPQVQLQQSGPGLVKPSQTLSLTCAI | 43 |
| | SGDSVSSDSAAWNWIRQSPSRGLEWLGRTYYRSKWYNDYAVSVKSR | |
| | ISINPDTSKNQFSLQLNSVTPEDTAVYYCARAQNNIAVAGFDYWGL | |
| | GTLVTVSSGILGSGGGGSGGGGSGGGGSQPVLTQSPSASGTPGQRV | |
| | TISCSGSSSNIGSNYVYWYQQLPGTAPTLLIYRNNORPSGVPDRFS | |
| | GSKSGTSASLAISGLRSEDEAEYYCAAWDDSLSGLYVFGTGTKVTV | |
| | LSGILGGGGSTTTPAPRPPTPAPTIASQPLSLRPEACRPAAGGAVH | |
| | TRGLDFACDIYIWAPLAGTCGVLLLSLVITLYCKRGRKKLLYIFKQ | |
| | PFMRPVQTTQEEDGCSCRFPEEEEGGCELRVKFSRSADAPAYQQGQ | |
| | NQLYNELNLGRREEYDVLDKRRGRDPEMGGKPRRKNPQEGLYNELQ | |
| | KDKMAEAYSEIGMKGERRRGKGHDGLYQGLSTATKDTYDALHMQAL | |
| | PPR | |
| LLm13-BBz | MLLLVTSLLLCELPHPAFLLIPQVQLVESGGNLVQPGGSLRLSCAA | 44 |
| | SGETFGSFSMSWVRQAPGGGLEWVAGLSARSSLTHYADSVKGRFTI | |
| | SRDNAKNSVYLQMNSLRVEDTAVYYCARRSYDSSGYWGHFYSYMDV | |
| | WGQGTLVTVSGSGGGGSGGGGSGGGGSSVLTQPSSVSAAPGQKVTI | |
| | SCSGSTSNIGNNYVSWYQQHPGKAPKLMIYDVSKRPSGVPDRFSGS | |
| | KSGNSASLDISGLQSEDEADYYCAAWDDSLSEFLFGTGTKLTVLGG | |
| | GGSVKGKHLCPSPLFPGPSKPFWVLVVVGGVLACYSLLVTVAFIIF | |
| | WVRSKRSRLLHSDYMNMTPRRPGPTRKHYQPYAPPRDFAAYRSRVK | |
| | FSRSADAPAYQQGQNQLYNELNLGRREEYDVLDKRRGRDPEMGGKP | |
| | RRKNPQEGLYNELQKDKMAEAYSEIGMKGERRRGKGHDGLYQGLST | |
| | ATKDTYDALHMQALPPRKRRKRGSGAAAEGRGSLLTCGDVEENPGP | |
| | SGMESDESGLPAMEIECRITGTLNGVEFELVGGGEGTPKQGRMTNK | |
| | MKSTKGALTFSPYLLSHVMGYGFYHEGTYPSGYENPFLHAINNGGY | |
| | TNTRIEKYEDGGVLHVSFSYRYEAGRVIGDFKVVGTGFPEDSVIFT | |
| | DKIIRSNATVEHLHPMGDNVLVGSFARTFSLRDGGYYSFVVDSHMH | |
| | FKSAIHPSILQNGGPMFAFRRVEELHSNTELGIVEYQHAFKTPTAF | |
| | ARSRAQSSNSAVDGTAGPGSTGSR | |
| LLm13-BBzΔGFP | MLLLVTSLLLCELPHPAFLLIPQVQLQQSGAGLVKPSQTLSLTCTI | 45 |
| | SGDSVSADRVAWNWIRQSPLRGLEWLGRIFYRSKWMVDYAVSVKSR | |
| | ISINPDTSKNQFSLQLNSVTPEDTAMYYCARATTRGYFDLWGRGTL | |
| | VTVSSGILGSGGGGSGGGGSGGGGSQPVLTQSPSASGTPGQRVTIS | |
| | CSGSSSNIGSNYVYWYQQLPGTAPKLLIYKNNQRPSGVPDRFSGSK | |
| | SGTAASLAISGLQSEDEADYYCAAWDDSLRGYVFGTGTKLTVLSGI | |
| | LGGGGSTTTPAPRPPTPAPTIASQPLSLRPEACRPAAGGAVHTRGL | |
| | DFACDIYIWAPLAGTCGVLLLSLVITLYCKRGRKKLLYIFKQPFMR | |
| | PVQTTQEEDGCSCRFPEEEEGGCELRVKFSRSADAPAYQQGQNQLY | |
| | NELNLGRREEYDVLDKRRGRDPEMGGKPRRKNPQEGLYNELQKDKM | |
| | AEAYSEIGMKGERRRGKGHDGLYQGLSTATKDTYDALHMQALPPR | |
| C4m18-BBz | MLLLVTSLLLCELPHPAFLLIPQVQLQQSGPGLVKPSPTLSLTCAI | 46 |
| | SGDSVSSNSAAWNWVRQSLSRGLEWLGRTYYRSKWYNGYAVSVRGR | |
| | ITTNADTSRNQFSLQLNSVTPEDTAVYYCARTGYSSSWVVNSNYWG | |
| | QGTLVTVSSGSASAPTGILGSGGGGSGGGGSGGGGSQPALTQSPSA | |
| | SGTPGQRVTISCSGSSSNIGSNYVYWYQQLPGTAPKLLIYKNNQRP | |
| | SGVPGRESGSKSGTAASLAISGLRSKDEADYYCAAWDDSLRGYVFG | |
| | TGTKLTVLGGGGSVKGKHLCPSPLFPGPSKPFWVLVVVGGVLACYS | |
| | LLVTVAFIIFWVRSKRSRLLHSDYMNMTPRRPGPTRKHYQPYAPPR | |
| | DFAAYRSRVKFSRSADAPAYQQGQNQLYNELNLGRREEYDVLDKRR | |
| | GRDPEMGGKPRRKNPQEGLYNELQKDKMAEAYSEIGMKGERRRGKG | |
| | HDGLYQGLSTATKDTYDALHMQALPPRKRRKRGSGAAAEGRGSLLT | |
| | CGDVEENPGPSGMESDESGLPAMEIECRITGTLNGVEFELVGGGEG | |
| | TPKQGRMTNKMKSTKGALTFSPYLLSHVMGYGFYHFGTYPSGYENP | |
| | FLHAINNGGYTNTRIEKYEDGGVLHVSFSYRYEAGRVIGDFKVVGT | |
| | GFPEDSVIFTDKIIRSNATVEHLHPMGDNVLVGSFARTFSLRDGGY | |
| | YSFVVDSHMHEKSAIHPSILQNGGPMFAFRRVEELHSNTELGIVEY | |
| | QHAFKTPIAFARSRAQSSNSAVDGTAGPGSTGSR | |
| C4m18-BBzΔGFP | MLLLVTSLLLCELPHPAFLLIPQVQLQQSGPGLVKPSPTLSLTCAI | 47 |
| | SGDSVSSNSAAWNWVRQSLSRGLEWLGRTYYRSKWYNGYAVSVRGR | |
| | ITTNADTSRNQFSLQLNSVTPEDTAVYYCARTGYSSSWVVNSNYWG | |
| | QGTLVTVSSGSASAPTGILGSGGGGSGGGGSGGGGSQPALTQSPSA | |
| | SGTPGQRVTISCSGSSSNIGSNYVYWYQQLPGTAPKLLIYKNNQRP | |
| | SGVPGRESGSKSGTAASLAISGLRSKDEADYYCAAWDDSLRGYVFG | |
| | TGTKLTVLGGGGSTTTPAPRPPTPAPTIASQPLSLRPEACRPAAGG | |
| | AVHTRGLDFACDIYIWAPLAGTCGVLLLSLVITLYCKRGRKKLLYI | |
| | FKQPFMRPVQTTQEEDGCSCRFPEEEEGGCELRVKFSRSADAPAYQ | |
| | QGQNQLYNELNLGRREEYDVLDKRRGRDPEMGGKPRRKNPQEGLYN | |
| | ELQKDKMAEAYSEIGMKGERRRGKGHDGLYQGLSTATKDTYDALHM | |
| | QALPPR | |

Exemplary amino acid sequences for CAR constructs comprising a CD28 costimulatory sequence and a CD3ζ signaling sequence are provided below in Table 6. The general design of the constructs shown in Table 6 is: CD8α signal peptide (SP)—anti-ASPH scFv—CD28 hinge-TM-costimulatory domain—CD3ζ signaling domain. 28z constructs comprise a GFP amino acid sequence linked via the T2A sequence to the C terminal end of the CAR construct. 28zΔGFP constructs that do not comprise the C-terminal GFP sequence are also contemplated.

TABLE 6
Exemplary 28z CAR constructs

| Construct | Amino Acid Sequence | SEQ ID: |
|---|---|---|
| 622-28z | MALPVTALLLPLALLLHAARPQVQLQQSGPGLVKPSQTLSLTCAIS | 48 |
| | GDSVSSNSAAWNWIRQSPSRGLEWLGRTYYRSKWYNDYAVSVKSRI | |
| | TINPDTSKNQFSLQLNSVTPEDTAVYYCARTGYSSSWVVNFDYWGQ | |
| | GTLVTVSSGSASAPTGILGSGGGGSGGGGSGGGGSQPVLTQSPSAS | |
| | GTPGQRVTISCSGSSSNIGSNYVYWYQQLPGTAPKLLIYKNNQRPS | |
| | GVPDRFSGSKSGTAASLAISGLQSEDEADYYCAAWDDSLRGYVFGT | |
| | GTKLTVLSGGGGSVKGKHLCPSPLFPGPSKPFWVLVVVGGVLACYS | |
| | LLVTVAFIIFWVRSKRSRLLHSDYMNMTPRRPGPTRKHYQPYAPPR | |
| | DFAAYRSRVKFSRSADAPAYQQGQNQLYNELNLGRREEYDVLDKRR | |
| | GRDPEMGGKPRRKNPQEGLYNELQKDKMAEAYSEIGMKGERRRGKG | |
| | HDGLYQGLSTATKDTYDALHMQALPPRKRRKRGSGAAAEGRGSLLT | |
| | CGDVEENPGPSGMESDESGLPAMEIECRITGTLNGVEFELVGGGEG | |
| | TPKQGRMTNKMKSTKGALTFSPYLLSHVMGYGFYHFGTYPSGYENP | |
| | FLHAINNGGYTNTRIEKYEDGGVLHVSFSYRYEAGRVIGDFKVVGT | |
| | GFPEDSVIFTDKIIRSNATVEHLHPMGDNVLVGSFARTFSLRDGGY | |
| | YSFVVDSHMHEKSAIHPSILQNGGPMFAFRRVEELHSNTELGIVEY | |
| | QHAFKTPIAFARSRAQSSNSAVDGTAGPGSTGSR | |
| 623-28z | MALPVTALLLPLALLLHAARPQVQLQQSGPGLVKPSQTLSLTCAIS | 49 |
| | GDSVSSDSAAWNWIRQSPSRGLEWLGRTYYRSKWYNDYAVSVKSRI | |
| | SINPDTSKNQFSLQLNSVTPEDTAVYYCARAQNNIAVAGFDYWGLG | |
| | TLVTVSSGILGSGGGGSGGGGSGGGGSQPVLTQSPSASGTPGQRVT | |
| | ISCSGSSSNIGSNYVYWYQQLPGTAPTLLIYRNNQRPSGVPDRFSG | |
| | SKSGTSASLAISGLRSEDEAEYYCAAWDDSLSGLYVFGTGTKVTVL | |
| | SGILGGGGSVKGKHLCPSPLFPGPSKPFWVLVVVGGVLACYSLLVT | |
| | VAFIIFWVRSKRSRLLHSDYMNMTPRRPGPTRKHYQPYAPPRDFAA | |
| | YRSRVKFSRSADAPAYQQGQNQLYNELNLGRREEYDVLDKRRGRDP | |
| | EMGGKPRRKNPQEGLYNELQKDKMAEAYSEIGMKGERRRGKGHDGL | |
| | YQGLSTATKDTYDALHMQALPPRKRRKRGSGAAAEGRGSLLTCGDV | |
| | EENPGPSGMESDESGLPAMEIECRITGTLNGVEFELVGGGEGTPKQ | |
| | GRMTNKMKSTKGALTFSPYLLSHVMGYGFYHFGTYPSGYENPFLHA | |
| | INNGGYTNTRIEKYEDGGVLHVSFSYRYEAGRVIGDFKVVGTGFPE | |
| | DSVIFTDKIIRSNATVEHLHPMGDNVLVGSFARTFSLRDGGYYSFV | |
| | VDSHMHFKSAIHPSILQNGGPMFAFRRVEELHSNTELGIVEYQHAF | |
| | KTPIAFARSRAQSSNSAVDGTAGPGSTGSR | |
| LLm13-28z | MLLLVTSLLLCELPHPAFLLIPQVQLVESGGNLVQPGGSLRLSCAA | 50 |
| | SGETFGSFSMSWVRQAPGGGLEWVAGLSARSSLTHYADSVKGRFTI | |
| | SRDNAKNSVYLQMNSLRVEDTAVYYCARRSYDSSGYWGHFYSYMDV | |
| | WGQGTLVTVSGSGGGGSGGGGSGGGGSSVLTQPSSVSAAPGQKVTI | |
| | SCSGSTSNIGNNYVSWYQQHPGKAPKLMIYDVSKRPSGVPDRFSGS | |
| | KSGNSASLDISGLQSEDEADYYCAAWDDSLSEFLFGTGTKLTVLGG | |
| | GGSVKGKHLCPSPLFPGPSKPFWVLVVVGGVLACYSLLVTVAFIIF | |
| | WVRSKRSRLLHSDYMNMTPRRPGPTRKHYQPYAPPRDFAAYRSRVK | |
| | FSRSADAPAYQQGQNQLYNELNLGRREEYDVLDKRRGRDPEMGGKP | |
| | RRKNPQEGLYNELQKDKMAEAYSEIGMKGERRRGKGHDGLYQGLST | |
| | ATKDTYDALHMQALPPRKRRKRGSGAAAEGRGSLLTCGDVEENPGP | |
| | SGMESDESGLPAMEIECRITGTLNGVEFELVGGGEGTPKQGRMTNK | |
| | MKSTKGALTFSPYLLSHVMGYGFYHEGTYPSGYENPFLHAINNGGY | |
| | TNTRIEKYEDGGVLHVSFSYRYEAGRVIGDFKVVGTGFPEDSVIFT | |
| | DKIIRSNATVEHLHPMGDNVLVGSFARTFSLRDGGYYSFVVDSHMH | |
| | FKSAIHPSILQNGGPMFAFRRVEELHSNTELGIVEYQHAFKTPTAF | |
| | ARSRAQSSNSAVDGTAGPGSTGSR | |
| C4m18-28z | MLLLVTSLLLCELPHPAFLLIPQVQLQQSGPGLVKPSPTLSLTCAI | 51 |
| | SGDSVSSNSAAWNWVRQSLSRGLEWLGRTYYRSKWYNGYAVSVRGR | |
| | ITTNADTSRNQFSLQLNSVTPEDTAVYYCARTGYSSSWVVNSNYWG | |
| | QGTLVTVSSGSASAPTGILGSGGGGSGGGGSGGGGSQPALTQSPSA | |
| | SGTPGQRVTISCSGSSSNIGSNYVYWYQQLPGTAPKLLIYKNNQRP | |
| | SGVPGRESGSKSGTAASLAISGLRSKDEADYYCAAWDDSLRGYVFG | |
| | TGTKLTVLGGGGSVKGKHLCPSPLFPGPSKPFWVLVVVGGVLACYS | |
| | LLVTVAFIIFWVRSKRSRLLHSDYMNMTPRRPGPTRKHYQPYAPPR | |
| | DFAAYRSRVKFSRSADAPAYQQGQNQLYNELNLGRREEYDVLDKRR | |
| | GRDPEMGGKPRRKNPQEGLYNELQKDKMAEAYSEIGMKGERRRGKG | |
| | HDGLYQGLSTATKDTYDALHMQALPPRKRRKRGSGAAAEGRGSLLT | |
| | CGDVEENPGPSGMESDESGLPAMEIECRITGTLNGVEFELVGGGEG | |
| | TPKQGRMTNKMKSTKGALTFSPYLLSHVMGYGFYHFGTYPSGYENP | |
| | FLHAINNGGYTNTRIEKYEDGGVLHVSFSYRYEAGRVIGDFKVVGT | |
| | GFPEDSVIFTDKIIRSNATVEHLHPMGDNVLVGSFARTFSLRDGGY | |
| | YSFVVDSHMHEKSAIHPSILQNGGPMFAFRRVEELHSNTELGIVEY | |
| | QHAFKTPIAFARSRAQSSNSAVDGTAGPGSTGSR | |

### Polypeptides

In some embodiments, the present disclosure provides anti-ASPH CAR polypeptides and fragments thereof. In particular embodiments, the CAR is an anti-ASPH CAR comprising an amino acid sequence as set forth in any one of SEQ ID NOs: 40-51.

“Polypeptide,” “peptide” and “protein” are used interchangeably, unless specified to the contrary, and according to conventional meaning, i.e., as a sequence of amino acids. Polypeptides are not limited to a specific length, e.g., they may comprise a full-length polypeptide or a polypeptide fragment, and may include one or more post-translational modifications of the polypeptide, for example, glycosylations, acetylations, phosphorylations and the like, as well as other modifications known in the art, both naturally occurring and non-naturally occurring. In various embodiments, the CAR polypeptides comprise a signal (or leader) sequence at the N-terminal end of the protein, which co-translationally or post-translationally directs transfer of the protein. Illustrative examples of suitable signal sequences useful in CARs contemplated in particular embodiments include, but are not limited to the IgG1 heavy chain signal polypeptide, a CD8α signal polypeptide (SEQ ID NO: 28), or a human GM-CSFR-α signal polypeptide (SEQ ID NO: 27). Polypeptides can be prepared using any of a variety of well-known recombinant and/or synthetic techniques.

### Polypeptide Variants

Polypeptides contemplated herein, encompass the CARs of the present disclosure, as well as functional variants thereof. The term “functional variant” as used herein refers to a CAR, polypeptide, or protein having substantial or significant sequence identity or similarity to a parent CAR, which functional variant retains the biological activity of the CAR of the parent CAR. Functional variants encompass, for example, CAR variants that retain the ability to recognize target cells to a similar extent, the same extent, or to a higher extent, as the parent CAR. Such variants may be naturally occurring or may be synthetically generated, for example, by modifying one or more of the above polypeptide sequences. For example, in particular embodiments, it may be desirable to improve the binding affinity and/or other biological properties of the CARs by introducing one or more substitutions, deletions, additions and/or insertions into a binding domain, hinge, TM domain, co-stimulatory domain or signaling domain of a CAR polypeptide. In particular embodiments, CAR polypeptides include polypeptides having at least about 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, or 99% amino acid identity to any of the CAR polypeptides described herein (e.g., any of SEQ ID NOs: 40-51), typically where the variant maintains at least one biological activity of the reference sequence.

As noted above, in particular embodiments, polypeptides may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

A functional variant can, for example, comprise the amino acid sequence of the parent CAR with at least one conservative amino acid substitution. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides contemplated in particular embodiments and still obtain a functional molecule that encodes a variant or derivative polypeptide with desirable characteristics. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc. Guidance in determining which amino acid residues can be substituted, inserted, or deleted without abolishing biological activity can be found using computer programs well known in the art, such as DNASTAR, DNA Strider, Geneious, Mac Vector, or Vector NTI software.

Alternatively or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

Polypeptide variants further include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

In one embodiment, where expression of two or more polypeptides is desired, the polynucleotide sequences encoding them can be separated by and IRES sequence as discussed elsewhere herein. In another embodiment, two or more polypeptides can be expressed as a fusion protein that comprises one or more self-cleaving polypeptide sequences, e.g., a 2A sequence shown in Table 7 (SEQ ID NOs: 39, 63-83).

### Fusion Polypeptides

Polypeptides contemplated in particular embodiments include fusion polypeptides (e.g., a CAR fusion protein). In some embodiments, fusion polypeptides and polynucleotides encoding fusion polypeptides are provided, e.g., CARs. Fusion polypeptides and fusion proteins refer to a polypeptide having at least two, three, four, five, six, seven, eight, nine, or ten or more polypeptide segments. Fusion polypeptides are typically linked C-terminus to N-terminus, although they can also be linked C-terminus to C-terminus, N-terminus to N-terminus, or N-terminus to C-terminus. The polypeptides of the fusion protein can be in any order or a specified order. Fusion polypeptides or fusion proteins can also include conservatively modified variants, polymorphic variants, alleles, mutants, subsequences, and interspecies homologs, so long as the desired transcriptional activity of the fusion polypeptide is preserved. Fusion polypeptides may be produced by chemical synthetic methods or by chemical linkage between the two moieties or may generally be prepared using other standard techniques. Ligated DNA sequences comprising the fusion polypeptide are operably linked to suitable transcriptional or translational control elements as discussed elsewhere herein.

In one embodiment, a fusion partner comprises a sequence that assists in expressing the protein (an expression enhancer) at higher yields than the native recombinant protein. Other fusion partners may be selected so as to increase the solubility of the protein or to enable the protein to be targeted to desired intracellular compartments or to facilitate transport of the fusion protein through the cell membrane.

In some embodiments, fusion polypeptides may further comprise a polypeptide cleavage signal between each of the polypeptide domains described herein. In addition, a polypeptide cleavage site can be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa, and enterokinase.

In some embodiments, the self-cleaving polypeptide site comprises a 2A or 2A-like site, sequence or domain (Donnelly et al., 2001. J. Gen. Virol. 82:1027-1041). Exemplary 2A sites are provided below in Table 7.

TABLE 7
Exemplary 2A sequences

| | |
|---|---|
| SEQ ID NO: 63 | GSGATNFSLLKQAGDVEENPGP |
| SEQ ID NO: 64 | ATNFSLLKQAGDVEENPGP |
| SEQ ID NO: 65 | LLKQAGDVEENPGP |
| SEQ ID NO: 66 | GSGEGRGSLLTCGDVEENPGP |
| SEQ ID NO: 39 | EGRGSLLTCGDVEENPGP |
| SEQ ID NO: 67 | LLTCGDVEENPGP |
| SEQ ID NO: 68 | GSGQCTNYALLKLAGDVESNPGP |
| SEQ ID NO: 69 | QCTNYALLKLAGDVESNPGP |
| SEQ ID NO: 70 | LLKLAGDVESNPGP |
| SEQ ID NO: 71 | GSGVKQTLNFDLLKLAGDVESNPGP |
| SEQ ID NO: 72 | VKQTLNFDLLKLAGDVESNPGP |
| SEQ ID NO: 73 | LLKLAGDVESNPGP |
| SEQ ID NO: 74 | LLNFDLLKLAGDVESNPGP |
| SEQ ID NO: 75 | TLNFDLLKLAGDVESNPGP |
| SEQ ID NO: 76 | LLKLAGDVESNPGP |
| SEQ ID NO: 77 | NFDLLKLAGDVESNPGP |
| SEQ ID NO: 78 | QLLNFDLLKLAGDVESNPGP |
| SEQ ID NO: 79 | APVKQTLNFDLLKLAGDVESNPGP |
| SEQ ID NO: 80 | VTELLYRMKRAETYCPRPLLAIHPTEARHKQKIVAPVKQT |
| SEQ ID NO: 81 | LNFDLLKLAGDVESNPGP |
| SEQ ID NO: 82 | LLAIHPTEARHKQKIVAPVKQTLNFDLLKLAGDVESNPGP |
| SEQ ID NO: 83 | EARHKQKIVAPVKQTLNFDLLKLAGDVESNPGP |

### Polynucleotides

In some embodiments, the present disclosure provides polynucleotides or nucleic acid molecules encoding one or more CAR polypeptides. As used herein, the terms “nucleotide” or “nucleic acid” refer to deoxyribonucleic acid (DNA), ribonucleic acid (RNA) and DNA/RNA hybrids. Polynucleotides may be single-stranded or double-stranded and either recombinant, synthetic, or isolated. Polynucleotides include, but are not limited to: pre-messenger RNA (pre-mRNA), messenger RNA (mRNA), RNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc.

In particular embodiments, polynucleotides may be codon-optimized. As used herein, the term “codon-optimized” refers to substituting codons in a polynucleotide encoding a polypeptide in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, (x) systematic variation of codon sets for each amino acid, (xi) isolated removal of spurious translation initiation sites and/or (xii) elimination of fortuitous polyadenylation sites otherwise leading to truncated RNA transcripts.

Terms used to describe sequence relationships between two or more polynucleotides or polypeptides include “reference sequence,” “comparison window,” “sequence identity,” “percentage of sequence identity,” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc, 1994-1998, Chapter 15.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted, or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence.

Moreover, it will be appreciated by those of ordinary skill in the art that, as a result of the degeneracy of the genetic code, there are many nucleotide sequences that encode a polypeptide, or fragment of variant thereof, as described herein. Some of these polynucleotides bear minimal homology to the nucleotide sequence of any native gene. Nonetheless, polynucleotides that vary due to differences in codon usage are specifically contemplated in particular embodiments, for example polynucleotides that are optimized for human and/or primate codon selection. Further, alleles of the genes comprising the polynucleotide sequences provided herein may also be used. Alleles are endogenous genes that are altered as a result of one or more mutations, such as deletions, additions and/or substitutions of nucleotides.

The polynucleotides contemplated herein, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed in particular embodiments, with the total length preferably being limited by the ease of preparation and use in the intended recombinant DNA protocol.

Provided herein is a nucleic acid molecule comprising a nucleotide sequence encoding an anti-ASPH CAR described herein and a suicide gene-encoding nucleotide sequence upstream or downstream of the nucleotide sequence encoding the CAR. In some embodiments, the suicide gene-encoding nucleotide sequence encodes an inducible human caspase (e.g., inducible human caspase-9 (iCasp9)).

Further provided herein is a nucleic acid molecule comprising a nucleotide sequence encoding an anti-ASPH CAR described herein and a nucleotide sequence encoding one, two or three cytokines. In some embodiments, the cytokine is one or more of human IL-15, IL-7, IL-12 or IL-21.

Also provided herein is a nucleic acid molecule comprising a nucleotide sequence encoding an anti-ASPH CAR described herein and a nucleotide sequence encoding one, two or three costimulatory molecules. In some embodiments, the costimulatory molecule is one or both of CD40-L or 4-1BB-L.

Further provided herein is a nucleic acid molecule comprising a nucleotide sequence encoding an anti-ASPH CAR described herein and a nucleotide sequence encoding one, two or three degrading enzymes.

Polynucleotides can be prepared, manipulated and/or expressed using any of a variety of well-established techniques known and available in the art.

### Vectors

In order to express a CAR described herein, an expression cassette encoding the CAR can be inserted into appropriate vector. The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA.

The term “expression cassette” as used herein refers to genetic sequences within a vector which can express a RNA, and subsequently a protein. The nucleic acid cassette contains the gene of interest, e.g., a CAR. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. In some embodiments, the nucleic acid cassette contains the sequence of a CAR used to increase the cytotoxicity of cancer cells that express ASPH. The cassette can be removed and inserted into a plasmid or viral vector as a single unit.

Exemplary vectors include, without limitation, plasmids, phagemids, cosmids, transposons, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4N5-DEST™, pLenti6/V5-DEST™, and pLenti6.2/V5-GW/lacZ (Invitrogen) for lentivirus-mediated gene transfer and expression in mammalian cells. In particular embodiments, the coding sequences of the CARs disclosed herein can be ligated into such expression vectors for the expression of the CARs in mammalian cells. In some embodiments, non-viral vectors are used to deliver one or more polynucleotides contemplated herein to a T cell.

In particular embodiments, the vector is a non-integrating vector, including but not limited to, an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally. The vector is engineered to harbor the sequence coding for the origin of DNA replication or “ori” from a lymphotrophic herpes virus or a gamma herpesvirus, an adenovirus, SV40, a bovine papilloma virus, or a yeast, specifically a replication origin of a lymphotrophic herpes virus or a gamma herpesvirus corresponding to oriP of EBV. In a particular aspect, the lymphotrophic herpes virus may be Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gamma herpesvirus. Typically, the host cell comprises the viral replication transactivator protein that activates the replication.

In particular embodiments, a polynucleotide is introduced into a target or host cell using a transposon vector system. In certain embodiments, the transposon vector system comprises a vector comprising transposable elements and a polynucleotide contemplated herein; and a transposase. In one embodiment, the transposon vector system is a single transposase vector system, see, e.g., WO 2008/027384. Exemplary transposases include, but are not limited to: piggyBac, Sleeping Beauty, Mos1, Tc1/mariner, Tol2, mini-Tol2, Tc3, MuA, Himar I, Frog Prince, and derivatives thereof. The piggyBac transposon and transposase are described, for example, in U.S. Pat. No. 6,962,810, which is incorporated herein by reference in its entirety. The Sleeping Beauty transposon and transposase are described, for example, in Izsvak et al., J. Mol. Biol. 302: 93-102 (2000), which is incorporated herein by reference in its entirety. The Tol2 transposon which was first isolated from the medaka fish Oryzias latipes and belongs to the hAT family of transposons is described in Kawakami et al. (2000). Mini-Tol2 is a variant of Tol2 and is described in Balciunas et al. (2006). The Tol2 and Mini-Tol2 transposons facilitate integration of a transgene into the genome of an organism when co-acting with the Tol2 transposase. The Frog Prince transposon and transposase are described, for example, in Miskey et al., Nucleic Acids Res. 31:6873-6881 (2003).

The “control elements” or “regulatory sequences” present in an expression vector are those non-translated regions of the vector (e.g., origin of replication, selection cassettes, promoters, enhancers, translation initiation signals (Shine Dalgarno sequence or Kozak sequence) introns, a polyadenylation sequence, 5′ and 3′ untranslated regions) which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, vectors include, but are not limited to expression vectors and viral vectors, will include exogenous, endogenous, or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

Illustrative ubiquitous promoters suitable for use in particular embodiments include, but are not limited to, a cytomegalovirus (CMV) immediate early promoter, a viral simian virus 40 (SV40) (e.g., early or late) promoter, a spleen focus forming virus (SFFV) promoter, a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1α) promoter, early growth response 1 (EGR1) promoter, a ferritin H (FerH) promoter, a ferritin L (FerL) promoter, a Glyceraldehyde 3-phosphate dehydrogenase (GAPDH) promoter, a eukaryotic translation initiation factor 4A1 (EIF4A1) promoter, a heat shock 70 kDa protein 5 (HSPA5) promoter, a heat shock protein 90 kDa beta, member 1 (HSP90B1) promoter, a heat shock protein 70 kDa (HSP70) promoter, a β-kinesin (β-KIN) promoter, the human ROSA 26 locus (Irions et al., Nature Biotechnology 25, 1477-1482 (2007)), a Ubiquitin C (UBC) promoter, a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, d1587rev primer-binding site substituted (MND) promoter (Challita et al., J Virol. 69(2):748-55 (1995)).

In some embodiments, the vectors described herein comprise an EF1α promoter operably linked to a polynucleotide encoding an anti-ASPH CAR. In some embodiments, the vectors described herein comprise an SFFV promoter operably linked to a polynucleotide encoding an anti-ASPH CAR.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest. Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Conditional expression can also be achieved by using a site-specific DNA recombinase. According to certain embodiments the vector comprises at least one (typically two) site(s) for recombination mediated by a site-specific recombinase. As used herein, the terms “recombinase” or “site specific recombinase” include excisive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use in particular embodiments include, but are not limited to: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. In particular embodiments, vectors include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides. In one embodiment, the IRES used in polynucleotides contemplated herein is an EMCV IRES.

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal polyA sequence (e.g., AATAAA, ATTAAA, AGTAAA). In particular embodiments, the poly(A) sequence is an SV40 polyA sequence, a bovine growth hormone polyA sequence (BGHpA), a rabbit β-globin polyA sequence (rβgpA), variants thereof, or another suitable heterologous or endogenous polyA sequence known in the art.

Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, polycation or lipid:nucleic acid conjugates, naked DNA, artificial virions, DEAE-dextran-mediated transfer, gene gun, and heat-shock.

Illustrative examples of polynucleotide delivery systems suitable for use in particular embodiments contemplated in particular embodiments include, but are not limited to, those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al. (2003) Gene Therapy. 10:180-187; and Balazs et al. (2011) Journal of Drug Delivery. 2011:1-12. Antibody-targeted, bacterially derived, non-living nanocell-based delivery is also contemplated in particular embodiments.

Viral vectors comprising polynucleotides contemplated in particular embodiments can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., mobilized peripheral blood, lymphocytes, bone marrow aspirates, tissue biopsy, etc.) or universal donor hematopoietic stem cells, followed by reimplantation of the cells into a patient.

In one embodiment, a viral vector comprising a polynucleotide encoding an anti-ASPH CAR is administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Illustrative examples of viral vector systems suitable for use in particular embodiments contemplated herein include, but are not limited to adeno-associated virus (AAV), retrovirus, herpes simplex virus, adenovirus, and vaccinia virus vectors.

### Genetically Modified Cells

In various embodiments, the present disclosure provides cells genetically modified to express the anti-ASPH CARs described herein. As used herein, the term “genetically engineered” or “genetically modified” refers to the addition of extra genetic material in the form of DNA or RNA into the total genetic material in a cell. As used herein, the term “gene therapy” refers to the introduction of extra genetic material in the form of DNA or RNA into the total genetic material in a cell that restores, corrects, or modifies expression of a gene, or for the purpose of expressing a therapeutic polypeptide, e.g., a CAR.

In particular embodiments, the present disclosure provides genetically modified cells and populations thereof comprising an anti-ASPH CAR. In some embodiments, the genetically modified cells comprise an anti-ASPH CAR and one or more additional exogenous transgenes.

In particular embodiments, the specificity of a primary immune effector cell is redirected to cells expressing ASPH, e.g., cancer cells, by genetically modifying the primary immune effector cell with a CAR contemplated herein. In various embodiments, a viral vector is used to genetically modify an immune effector cell with a particular polynucleotide encoding a CAR comprising an anti-ASPH antigen binding domain that binds an ASPH polypeptide; a hinge domain; a transmembrane (TM) domain, a short oligo- or polypeptide linker, that links the TM domain to the intracellular signaling domain of the CAR; and one or more intracellular co-stimulatory domains; and a signaling domain.

### Immune Effector Cells

In particular embodiments, the present disclosure provides genetically modified cells (e.g., immune effector cells) and populations thereof comprising an anti-ASPH CAR. In such embodiments, the anti-ASPH CARs contemplated herein are introduced and expressed in immune effector cells so as to redirect the specificity of the immune cell to a target antigen of interest, e.g., a ASPH polypeptide. In some embodiments, the genetically modified cell expresses an anti-ASPH CAR on the cell surface. In some embodiments, the genetically modified immune effector cells comprise an anti-ASPH CAR and one or more additional exogenous transgenes.

An “immune effector cell,” is any cell of the immune system that has one or more effector functions (e.g., cytotoxic cell killing activity, secretion of cytokines, induction of ADCC and/or CDC). Exemplary immune effector cells include T lymphocytes, in particular cytotoxic T cells (CTLs; CD8+ T cells), TILs, and helper T cells (HTLs; CD4+ T cells), natural killer (NK) cells, and natural killer T (NKT) cells. Immune effector cells also include progenitors of effector cells wherein such progenitor cells can be induced to differentiate into immune effector cells in vivo or in vitro. In some embodiments, an immune effector cell is a Natural Killer (NK)-like cell, a hematopoietic progenitor cell, a peripheral blood (PB) derived T cell or an umbilical cord blood (UCB) derived T cell.

In particular embodiments, anti-ASPH CAR-modified immune effector cells comprise T cells. The terms “T cell” or “T lymphocyte” are art-recognized and are intended to include thymocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), CD4⁺CD8⁺ T cell, CD4⁻CD8⁻ T cell, or any other subset of T cells. Other illustrative populations of T cells suitable for use in particular embodiments include naive T cells and memory T cells.

In some embodiments, the T cells are derived from a mammalian subject. In some embodiments, the T cells are derived from a primate subject, such as a human subject. T cells can be obtained from a number of sources including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation. In one embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. As would be appreciated by those of ordinary skill in the art, a washing step may be accomplished by methods known to those in the art, such as by using a semiautomated flowthrough centrifuge. For example, the Cobe 2991 cell processor, the Baxter CytoMate, or the like. After washing, the cells may be resuspended in a variety of biocompatible buffers or other saline solution with or without buffer. In certain embodiments, the undesirable components of the apheresis sample may be removed in the cell directly resuspended culture media.

In some embodiments, a population of cells comprising T cells, e.g., peripheral blood mononuclear cells (PBMCs), is genetically modified according to the present disclosure. In some embodiments, the population of PBMCs is not subjected to positive or negative selection prior to activation, expansion, and/or genetic modification. In other embodiments, T cells are isolated or purified from PBMCs prior to activation, expansion, and/or genetic modification. In such embodiments, the population of PBMCs can be treated to lyse the red blood cells and deplete the monocytes, for example, by centrifugation through a PERCOLL™ gradient. In some embodiments, cytotoxic and/or helper T lymphocytes are isolated from PBMCs. In some embodiments, the isolated T cells can be sorted into naïve, memory, and effector T cell subpopulations either before or after activation, expansion, and/or genetic modification. In certain embodiments, specific subpopulation of T cells, expressing one or more of the following markers: CD3, CD4, CD8, CD28, CD45RA, CD45RO, CD62, CD127, and HLA-DR can be further isolated by positive or negative selection techniques.

In some embodiments, immune effector cells include progenitors of immune effectors cells such as hematopoietic stem cells (HSCs) contained within the CD34⁺ population of cells derived from cord blood, bone marrow, or mobilized peripheral blood, and which differentiate into mature immune effector cells upon administration in a subject, or which can be induced in vitro to differentiate into mature immune effector cells.

In some embodiments, a cell expresses an anti-ASPH CAR disclosed herein and additionally expresses one, two or three inhibitors of an immune checkpoint molecule. In some cases, the immune checkpoint molecule is PD1, PD-L1, PD-L2, CTLA4, TIM3, CEACAM-1, CEACAM-3, CEACAM-5, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MEW class I, MEW class II, GALS, adenosine or TGFR.

In some embodiments, a cell expresses an anti-ASPH CAR disclosed herein and additionally expresses a PD-1 dominant negative receptor. In some embodiments, a cell expresses an anti-ASPH CAR disclosed herein and additionally expresses an apoptosis-inducing agent. In some embodiments, the apoptosis-inducing agent is a TRAILR2 agonist.

In some embodiments, a cell expresses an anti-ASPH CAR disclosed herein and additionally expresses one, two or three cytokines. In some embodiments, the cytokine is one or more of human IL-15, IL-7, IL-12 or IL-21. Also provided herein are CAR T cells redirected for universal cytokine killing (TRUCKs). See, Schubert et al., Int. J. Cancer, 142:1738-1747 (2017). In some embodiments, a cell expresses an anti-ASPH CAR disclosed herein and a CAR-inducible IL-12 cytokine cassette under the transcriptional control of a nuclear factor of activated T cells (NFAT) promoter within the CAR construct. Id.

In some embodiments, a cell expresses an anti-ASPH CAR disclosed herein and additionally expresses one, two or three costimulatory molecules. In some embodiments, the costimulatory molecule is one or both of CD40-L or 4-1BB-L.

In some embodiments, a cell expresses an anti-ASPH CAR disclosed herein and additionally expresses one, two or three degrading enzymes.

In some embodiments, a cell expresses an anti-ASPH CAR disclosed herein and does not express endogenous T cell receptor (TCR) and/or Human Leukocyte Antigen (HLA) molecules, in order to prevent graft-versus-host disease (GvHD) or rejection. See, Schubert et al., Int. J. Cancer, 142:1738-1747 (2017). In some embodiments, endogenous TCR and/or HLA molecule expression can be eliminated by gene-editing systems (for example, CRISPR, TALEN or ZFN).

### Genetic Modifications

In some embodiments, the genetically modified cells described herein comprise an anti-ASPH CAR and further comprise one or more additional exogenous transgenes. In some embodiments, the one or more additional exogenous transgenes encode a detectable tag, a safety-switch system, or a chimeric switch receptor.

### Detectable Tags

In some embodiments, the genetically modified cells described herein comprise an anti-ASPH CAR and further comprise an exogenous transgene encoding a detectable tag. Examples of detectable tags include but are not limited to, FLAG tags, poly-histidine tags (e.g. 6×His), SNAP tags, Halo tags, cMyc tags, glutathione-S-transferase tags, avidin, enzymes, fluorescent proteins, luminescent proteins, chemiluminescent proteins, bioluminescent proteins, and phosphorescent proteins.

In some embodiments the fluorescent protein is selected from the group consisting of blue/UV proteins (such as BFP, TagBFP, mTagBFP2, Azurite, EBFP2, mKalama1, Sirius, Sapphire, and T-Sapphire); cyan proteins (such as CFP, eCFP, Cerulean, SCFP3A, mTurquoise, mTurquoise2, monomeric Midoriishi-Cyan, TagCFP, and mTFP1); green proteins (such as: GFP, eGFP, meGFP (A208K mutation), Emerald, Superfolder GFP, Monomeric Azami Green, TagGFP2, mUKG, mWasabi, Clover, and mNeonGreen); yellow proteins (such as YFP, eYFP, Citrine, Venus, SYFP2, and TagYFP); orange proteins (such as Monomeric Kusabira-Orange, mKOK, mKO2, mOrange, and mOrange2); red proteins (such as RFP, mRaspberry, mCherry, mStrawberry, mTangerine, tdTomato, TagRFP, TagRFP-T, mApple, mRuby, and mRuby2); far-red proteins (such as mPlum, HcRed-Tandem, mKate2, mNeptune, and NirFP); near-infrared proteins (such as TagRFP657, IFP1.4, and iRFP); long stokes shift proteins (such as mKeima Red, LSS-mKate1, LSS-mKate2, and mBeRFP); photoactivatible proteins (such as PA-GFP, PAmCherry1, and PATagRFP); photoconvertible proteins (such as Kaede (green), Kaede (red), KikGR1 (green), KikGR1 (red), PS-CFP2, PS-CFP2, mEos2 (green), mEos2 (red), mEos3.2 (green), mEos3.2 (red), PSmOrange, and PSmOrange); and photoswitchable proteins (such as Dronpa). In some embodiments, the detectable tag can be selected from AmCyan, AsRed, DsRed2, DsRed Express, E2-Crimson, HcRed, ZsGreen, ZsYellow, mCherry, mStrawberry, mOrange, mBanana, mPlum, mRaspberry, tdTomato, DsRed Monomer, and/or AcGFP, all of which are available from Clontech.

In some embodiments, the detectable tag and the anti-ASPH CAR are expressed from the same expression cassette. For example, in some embodiments, the genetically modified cells described herein comprise an expression cassette comprising a first polynucleotide sequence encoding an anti-ASPH CAR and a second polynucleotide sequence encoding a detectable tag. In some embodiments, the first polynucleotide sequence encodes an anti-ASPH CAR that is at least 90%, 95%, 96%, 97%, 98%, or 99% identical to one of SEQ ID NOs: 41, 43, 45, 47, 48-51 and the second polynucleotide sequence encodes a GFP protein (e.g. SEQ ID NO: 38).

### Safety Switches

In some embodiments, the modified immune effector cells described herein further comprise an exogenous transgene encoding a safety-switch system. Safety-switch systems (also referred to in the art as suicide gene systems) comprise exogenous transgenes encoding for one or more proteins that enable the elimination of a modified immune effector cell after the cell has been administered to a subject. Examples of safety-switch systems are known in the art. For example, safety-switch systems include genes encoding for proteins that convert non-toxic pro-drugs into toxic compounds such as the Herpes simplex thymidine kinase (Hsv-tk) and ganciclovir (GCV) system (Hsv-tk/GCV). Hsv-tk converts non-toxic GCV into a cytotoxic compound that leads to cellular apoptosis. As such, administration of GCV to a subject that has been treated with modified immune effector cells comprising a transgene encoding the Hsv-tk protein can selectively eliminate the modified immune effector cells while sparing endogenous immune effector cells. (See e.g., Bonini et al., Science, 1997, 276(5319):1719-1724; Ciceri et al., Blood, 2007, 109(11):1828-1836; Bondanza et al., Blood 2006, 107(5):1828-1836).

Additional safety-switch systems include genes encoding for cell-surface markers, enabling elimination of modified immune effector cells by administration of a monoclonal antibody specific for the cell-surface marker via ADCC. In some embodiments, the cell-surface marker is CD20 and the modified immune effector cells can be eliminated by administration of an anti-CD20 monoclonal antibody such as Rituximab (See e.g., Introna et al., Hum Gene Ther, 2000, 11(4):611-620; Serafini et al., Hum Gene Ther, 2004, 14, 63-76; van Meerten et al., Gene Ther, 2006, 13, 789-797). Similar systems using EGF-R and Cetuximab or Panitumumab are described in WO 2018/006880.

Additional safety-switch systems include transgenes encoding one or more suicide genes that sensitize the genetically modified cells to apoptosis induced by administration of an exogenous agent. Exemplary suicide genes include pro-apoptotic molecules comprising one or more binding sites for a chemical inducer of dimerization (CID), enabling elimination of the genetically modified cells by administration of a CID which induces oligomerization of the pro-apoptotic molecules and activation of the apoptosis pathway. In some embodiments, the pro-apoptotic molecule is Fas (also known as CD95) (Thomis et al., Blood, 2001, 97(5), 1249-1257). In some embodiments, the pro-apoptotic molecule is Caspase-9 (Straathof et al., Blood, 2005, 105(11), 4247-4254), Caspase-8, or cytosine deaminase.

### Switch Receptors

In some embodiments, the genetically modified cells described herein further comprise an exogenous transgene encoding a chimeric switch receptor. Chimeric switch receptors are engineered cell-surface receptors comprising an extracellular domain from an endogenous cell-surface receptor and a heterologous intracellular signaling domain, such that ligand recognition by the extracellular domain results in activation of a different signaling cascade than that activated by the wild type form of the cell-surface receptor.

In some embodiments, the chimeric switch receptor comprises the extracellular domain of an inhibitory cell-surface receptor fused to an intracellular domain that leads to the transmission of an activating signal rather than the inhibitory signal normally transduced by the inhibitory cell-surface receptor. In particular embodiments, extracellular domains derived from cell-surface receptors known to inhibit immune effector cell activation can be fused to activating intracellular domains. Engagement of the corresponding ligand will then activate signaling cascades that increase, rather than inhibit, the activation of the immune effector cell. For example, in some embodiments, the modified immune effector cells described herein comprise a transgene encoding a PD1-CD28 switch receptor, wherein the extracellular domain of PD1 is fused to the intracellular signaling domain of CD28 (See e.g., Liu et al., Cancer Res 76:6 (2016), 1578-1590 and Moon et al., Molecular Therapy 22 (2014), S201). In some embodiments, the modified immune effector cells described herein comprise a transgene encoding the extracellular domain of CD200R and the intracellular signaling domain of CD28 (See Oda et al., Blood 130:22 (2017), 2410-2419).

### Manufacturing Methods

In some embodiments, the present disclosure provides methods for making genetically modified immune cells expressing an anti-ASPH CAR. In some embodiments, the method comprises introducing a vector comprising a nucleic acid encoding an anti-ASPH CAR (e.g., an expression cassette encoding an anti-ASPH CAR) to a population of immune effector cells with such that the immune effector cells express the anti-ASPH CAR. In particular embodiments, the immune effector cells are obtained from a subject prior to in vitro manipulation or genetic modification. The immune effector cells can be autologous/autogeneic (“self”) or non-autologous (“non-self,” e.g., allogeneic, syngeneic or xenogeneic).

In certain embodiments, the immune effector cells genetically modified without further manipulation in vitro. In some embodiments, the immune effector cells are first activated and/or expanded in vitro prior to being genetically modified to express an anti-ASPH CAR. In some embodiments, the immune effector cells are genetically modified to express an anti-ASPH CAR and are then activated and/or expanded in vitro. In some embodiments, the expression vector further encodes a detectable label such that successfully transduced cells carrying the expression vector can be sorted and isolated using flow cytometry. These successfully transduced cells can then be further propagated to increase the number of these CAR protein expressing cells. Standard procedures are used for cryopreservation of immune effector cells expressing the CAR protein for storage and/or preparation for use in a human subject. In one embodiment, the in vitro transduction, culture and/or expansion of immune effector cells are performed in the absence of non-human animal derived products such as fetal calf serum and fetal bovine serum.

Manufacturing methods contemplated herein may further comprise cryopreservation of modified immune cells for storage and/or preparation for use in a human subject. As used herein, “cryopreserving,” refers to the preservation of cells by cooling to sub-zero temperatures, such as (typically) 77 K or −196° C. (the boiling point of liquid nitrogen).

In some embodiments, a method of storing genetically modified murine, human, or humanized CAR protein expressing immune effector cells which target an ASPH expressing cell, comprises cryopreserving the immune effector cells such that the cells remain viable upon thawing. A fraction of the immune effector cells expressing the CAR proteins can be cryopreserved by methods known in the art to provide a permanent source of such cells for the future treatment of patients afflicted with an ASPH expressing cancer cell. When needed, the cryopreserved transformed immune effector cells can be thawed, grown and expanded for more such cells.

Cryoprotective agents are often used at sub-zero temperatures to prevent the cells being preserved from damage due to freezing at low temperatures or warming to room temperature. Cryopreservative agents and optimal cooling rates can protect against cell injury. Cryoprotective agents which can be used include but are not limited to dimethyl sulfoxide (DMSO) (Lovelock and Bishop, Nature, 1959; 183: 1394-1395; Ashwood-Smith, Nature, 1961; 190: 1204-1205), glycerol, polyvinylpyrrolidine (Rinfret, Ann. N.Y. Acad. Sci., 1960; 85: 576), and polyethylene glycol (Sloviter and Ravdin, Nature, 1962; 196: 48).

In some embodiments, the present disclosure provides methods for producing genetically modified T cells comprising introducing a vector comprising a nucleic acid encoding an anti-ASPH CAR (e.g., an expression cassette encoding an anti-ASPH CAR) to a population of T cells. In some embodiments, the T cells can be activated and expanded before or after genetic modification to express a CAR, using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells modified to express an anti-ASPH CAR can be activated and expanded before and/or after the T cells are modified.

In some embodiments, the expression vector further encodes a detectable label such that successfully transduced T cells carrying the expression vector can be sorted and isolated using flow cytometry. These successfully transduced T cells can then further propagated to increase the number of these CAR protein expressing cells and can be activated using anti-CD3 antibodies and/or anti-CD28 antibodies and IL-2 or any other methods known in the art as described elsewhere herein.

In various embodiments, a method for manufacturing T cells contemplated herein comprises activating a population of cells comprising T cells and expanding the population of T cells. T cell activation can be accomplished by providing a primary stimulation signal through the T cell TCR/CD3 complex or via stimulation of the CD2 surface protein and by providing a secondary costimulation signal through an accessory molecule, e.g, CD28.

In some embodiments, the TCR/CD3 complex may be stimulated by contacting the T cell with a suitable CD3 binding agent, e.g., a CD3 ligand or an anti-CD3 monoclonal antibody. Illustrative examples of CD3 antibodies include, but are not limited to, OKT3, G19-4, BC3, CRIS-7 and 64.1. In some embodiments, a CD2 binding agent may be used to provide a primary stimulation signal to the T cells. Illustrative examples of CD2 binding agents include, but are not limited to, CD2 ligands and anti-CD2 antibodies, e.g., the T11.3 antibody in combination with the T11.1 or T11.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100).

In addition to the primary stimulation signal provided through the TCR/CD3 complex, or via CD2, induction of T cell responses requires a second, costimulatory signal provided by a costimulatory ligand that specifically binds a cognate costimulatory molecule on a T cell, thereby providing a signal which, in addition to the primary signal provided by, for instance, binding of a TCR/CD3 complex, mediates a desired T cell response. Suitable costimulatory ligands include, but are not limited to, CD7, B7-1 (CD80), B7-2 (CD86), 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, ILT3, ILT4, an agonist or antibody that binds Toll ligand receptor, and a ligand that specifically binds with B7-H3.

In some embodiments, a costimulatory ligand comprises an antibody or antigen binding fragment thereof that specifically binds to a costimulatory molecule present on a T cell, including but not limited to, CD27, CD28, 4-1BB, OX40, CD30, CD40, ICOS, lymphocyte function-associated antigen-1 (LFA-1), CD7, LIGHT, NKG2C, B7-H3, and a ligand that specifically binds with CD83.

In particular embodiments, a CD28 binding agent can be used to provide a costimulatory signal. Illustrative examples of CD28 binding agents include but are not limited to: natural CD28 ligands, e.g., a natural ligand for CD28 (e.g., a member of the B7 family of proteins, such as B7-1 (CD80) and B7-2 (CD86); and anti-CD28 monoclonal antibody or fragment thereof capable of crosslinking the CD28 molecule, e.g., monoclonal antibodies 9.3, B-T3, XR-CD28, KOLT-2, 15E8, 248.23.2, and EX5.3D10.

In some embodiments, the costimulatory signal is provided by a costimulatory ligand presented on an antigen presenting cell, such as an artificial APC (aAPC). Artificial APCs can be made by engineering K562, U937, 721.221, T2, or C1R cells to stably express and/or secrete of a variety of costimulatory molecules and cytokines to support ex vivo growth and long-term expansion of genetically modified T cells. In a particular embodiment, K32 or U32 aAPCs are used to direct the display of one or more antibody-based stimulatory molecules on the aAPC cell surface. Populations of T cells can be expanded by aAPCs expressing a variety of costimulatory molecules including, but not limited to, CD137L (4-1BBL), CD134L (OX40L), and/or CD80 or CD86. Exemplary aAPCs are provided in WO 03/057171 and US2003/0147869, incorporated by reference in their entireties.

In certain embodiments, binding agents that provide stimulatory and costimulatory signals are localized a solid surface (e.g., a bead or a plate). In certain embodiments, binding agents that provide stimulatory and costimulatory signals are localized on the surface of a cell. This can be accomplished by transfecting or transducing a cell with a nucleic acid encoding the binding agent in a form suitable for its expression on the cell surface or alternatively by coupling a binding agent to the cell surface. In a particular embodiment, the binding agents that provide stimulatory and costimulatory signals are both provided in a soluble form (provided in solution). In some embodiments, the methods for manufacturing T cells contemplated herein comprise activating T cells with anti-CD3 and anti-CD28 antibodies.

In some embodiments, T cells are expanded after activation. T cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion. In one embodiment, expanding T cells activated by the methods contemplated herein further comprises culturing a population of cells comprising T cells for several hours (about 3 hours) to about 7 days to about 28 days or any hourly integer value in between. In another embodiment, the T cell composition may be cultured for 14 days. In a particular embodiment, T cells are cultured for about 21 days. In another embodiment, the T cell compositions are cultured for about 2-3 days. Several cycles of stimulation/activation/expansion may also be desired such that culture time of T cells can be 60 days or more.

In particular embodiments, conditions appropriate for T cell culture include an appropriate media (e.g., Minimal Essential Media or RPMI Media 1640 or, X-vivo 15, (Lonza)) and one or more factors necessary for proliferation and viability including, but not limited to, serum (e.g., fetal bovine or human serum), interleukin-2 (IL-2), insulin, IFN-γ, IL-4, IL-7, IL-21, GM-CSF, IL-10, IL-12, IL-15, TGFβ, and TNF-α or any other additives suitable for the growth of cells known to the skilled artisan.

Further illustrative examples of cell culture media include, but are not limited to RPMI 1640, Clicks, AIM-V, DMEM, MEM, a-MEM, F-12, X-Vivo 1 5, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Illustrative examples of other additives for T cell expansion include, but are not limited to, surfactant, piasmanate, pH buffers such as HEPES, and reducing agents such as N-acetyl-cysteine and 2-mercaptoethanol

Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

### Compositions and Formulations

The compositions contemplated herein may comprise one or more anti-ASPH CAR polypeptides, polynucleotides encoding anti-ASPH CAR polypeptides, vectors comprising same, and/or genetically modified immune effector cells comprising anti-ASPH CAR polypeptides. Compositions include, but are not limited to pharmaceutical compositions. A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular embodiments, the compositions comprise an effective amount of anti-ASPH CAR-expressing immune effector cells. As used herein, “an effective amount” of a genetically modified cell, e.g., T cell, is the amount of cells required to achieve a beneficial or desired prophylactic or therapeutic result, including clinical results. The effective amount of a genetically modified therapeutic cell may vary according to factors such as the disease state, age, sex, and weight of the individual and includes an amount that is effective to “treat” a subject. The effective amount of the compositions described herein suitable for administration to a subject can be determined by a physician with consideration of individual differences in age, weight, extent of disease, and condition of the subject.

It can generally be stated that a pharmaceutical composition comprising the T cells described herein may be administered at a dosage of 10² to 10¹⁰ cells/kg body weight, preferably 10⁵ to 10⁶ cells/kg body weight, including all integer values within those ranges. The number of cells will depend upon the ultimate use for which the composition is intended as will the type of cells included therein. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. CAR expressing cell compositions may be administered multiple times at dosages within these ranges. The cells may be allogeneic, syngeneic, xenogeneic, or autologous to the patient undergoing therapy. If desired, the treatment may also include administration of mitogens (e.g., PHA), cytokines, and/or chemokines (e.g., IFN-γ, IL-2, IL-12, TNF-alpha, IL-18, and TNF-beta, GM-CSF, IL-4, IL-13, Flt3-L, RANTES, MIP1α, etc.) as described herein to enhance induction of the immune response.

Generally, compositions comprising the cells activated and expanded as described herein may be utilized in the treatment and prevention of diseases that arise in individuals who are immunocompromised. In particular embodiments, compositions comprising the CAR-modified T cells contemplated herein are used in the treatment of cancer. The CAR-modified T cells may be administered either alone, or as a pharmaceutical composition in combination with carriers, diluents, excipients, and/or with other components such as IL-2 or other cytokines or cell populations. In particular embodiments, pharmaceutical compositions comprise an amount of genetically modified T cells, in combination with one or more pharmaceutically or physiologically acceptable carriers, diluents or excipients.

Pharmaceutical compositions comprising a CAR-expressing immune effector cell population, such as T cells, may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions are preferably formulated for parenteral administration, e.g., intravascular (intravenous or intraarterial), intraperitoneal or intramuscular administration.

The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In one embodiment, the T cell compositions contemplated herein are formulated in a pharmaceutically acceptable cell culture medium. Such compositions are suitable for administration to human subjects. In particular embodiments, the pharmaceutically acceptable cell culture medium is a serum free medium. Serum-free medium has several advantages over serum containing medium, including a simplified and better defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein. Illustrative examples of serum-free media used in particular compositions includes, but is not limited to QBSF-60 (Quality Biological, Inc.), StemPro-34 (Life Technologies), and X-VIVO 10.

In some embodiments, compositions comprising immune effector cells contemplated herein are formulated in a solution comprising a cryopreservation medium. For example, cryopreservation media with cryopreservation agents may be used to maintain a high cell viability outcome post-thaw. Illustrative examples of cryopreservation media used in particular compositions includes, but is not limited to, CryoStor CS10, CryoStor CSS, and CryoStor CS2.

In a particular embodiment, compositions comprise an effective amount of CAR-expressing immune effector cells, alone or in combination with one or more therapeutic agents. Thus, the CAR-expressing immune effector cell compositions may be administered alone or in combination with other known cancer treatments, such as radiation therapy, chemotherapy, transplantation, immunotherapy, hormone therapy, photodynamic therapy, etc. In some embodiments, the CAR-expressing immune effector cells may be engineered to be immune or weakly affected by the chemotherapy. The compositions may also be administered in combination with antibiotics. Such therapeutic agents may be accepted in the art as a standard treatment for a particular disease state as described herein, such as a particular cancer. Exemplary therapeutic agents contemplated in particular embodiments include cytokines, growth factors, steroids, NSAID s, DMARDs, anti-inflammatories, chemotherapeutics, radiotherapeutics, therapeutic antibodies, immune checkpoint inhibitors, or other active and ancillary agents.

In certain embodiments, compositions comprising CAR-expressing immune effector cells disclosed herein may be administered in conjunction with any number of chemotherapeutic agents. Illustrative examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on cancers such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

A variety of other therapeutic agents may be used in conjunction with the compositions described herein. In one embodiment, the composition comprising CAR-expressing immune effector cells is administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

Other exemplary NSAIDs are chosen from the group consisting of ibuprofen, naproxen, naproxen sodium, Cox-2 inhibitors such as VIOXX® (rofecoxib) and CELEBREX® (celecoxib), and sialylates. Exemplary analgesics are chosen from the group consisting of acetaminophen, oxycodone, tramadol of proporxyphene hydrochloride. Exemplary glucocorticoids are chosen from the group consisting of cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

Illustrative examples of therapeutic antibodies suitable for combination with the CAR modified T cells contemplated in particular embodiments, include but are not limited to, atezolizumab, avelumab, bavituximab, bevacizumab (avastin), bivatuzumab, blinatumomab, conatumumab, crizotinib, daratumumab, duligotumab, dacetuzumab, dalotuzumab, durvalumab, elotuzumab (HuLuc63), gemtuzumab, ibritumomab, indatuximab, inotuzumab, ipilimumab, lorvotuzumab, lucatumumab, milatuzumab, moxetumomab, nivolumab, ocaratuzumab, ofatumumab, pembrolizumab, rituximab, siltuximab, teprotumumab, and ublituximab.

In certain embodiments, the compositions described herein are administered in conjunction with a cytokine. By “cytokine” as used herein is meant a generic term for proteins released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and —II; erythropoietin (EPO); osteoinductive factors; interferons (IFN) such as IFNα, IFNβ, IFNγ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12, IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In certain embodiments, the compositions described herein are administered in conjunction with an immune checkpoint inhibitor. Several immune checkpoint inhibitors are known in the art and have received FDA approval for the treatment of one or more cancers. For example, FDA-approved PD-L1 inhibitors include Atezolizumab (Tecentriq®, Genentech), Avelumab (Bavencio®, Pfizer), and Durvalumab (Imfinzi®, AstraZeneca); FDA-approved PD-1 inhibitors include Pembrolizumab (Keytruda®, Merck) and Nivolumab (Opdivo®, Bristol-Myers Squibb); and FDA-approved CTLA4 inhibitors include Ipilimumab (Yervoy®, Bristol-Myers Squibb). Additional inhibitory immune checkpoint molecules that may be the target of future therapeutics include A2AR, B7-H3, B7-H4, BTLA, IDO, LAG3 (e.g., BMS-986016, under development by BSM), KIR (e.g., Lirilumab, under development by BSM), TIM3, TIGIT, and VISTA.

### Therapeutic Methods

In some embodiments, the present disclosure provides methods of treating a cancer comprising administration of the genetically modified immune effector cells comprising anti-ASPH CARs to a subject in need thereof. As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction the disease or condition, or the delaying of the progression of the disease or condition. “Treatment” does not necessarily indicate complete eradication or cure of the disease or condition, or associated symptoms thereof.

As used herein, the terms “individual” and “subject” are often used interchangeably and refer to any animal that exhibits a symptom of a disease, disorder, or condition that can be treated with the gene therapy vectors, cell-based therapeutics, and methods contemplated elsewhere herein. In some embodiments, a subject includes any animal that exhibits symptoms of a disease, disorder, or condition related to cancer that can be treated with the gene therapy vectors, cell-based therapeutics, and methods contemplated elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include human patients that have an ASPH expressing cancer, have been diagnosed with an ASPH expressing cancer, or are at risk of having an ASPH expressing cancer.

As used herein, the term “cancer” relates generally to a class of diseases or conditions in which abnormal cells divide without control and can invade nearby tissues. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

By “enhance” or “promote,” or “increase” or “expand” refers generally to the ability of a composition contemplated herein, e.g., a genetically modified T cell or vector encoding a CAR, to produce, elicit, or cause a greater physiological response (i.e., downstream effects) compared to the response caused by either vehicle or a control molecule/composition. A measurable physiological response may include an increase in T cell expansion, activation, persistence, and/or an increase in cancer cell killing ability, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. A “decrease” or “reduced” amount is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

The genetically modified immune effector cells contemplated herein provide methods of adoptive immunotherapy for use in the prevention, treatment, and/or amelioration of cancers that express ASPH or for preventing, treating, and/or ameliorating at least one symptom associated with an ASPH expressing cancer. As used herein, the phrase “ameliorating at least one symptom of” refers to decreasing one or more symptoms of the disease or condition for which the subject is being treated. In particular embodiments, the disease or condition being treated is a cancer, wherein the one or more symptoms ameliorated include, but are not limited to, weakness, fatigue, shortness of breath, easy bruising and bleeding, frequent infections, enlarged lymph nodes, distended or painful abdomen (due to enlarged abdominal organs), bone or joint pain, fractures, unplanned weight loss, poor appetite, night sweats, persistent mild fever, and decreased urination (due to impaired kidney function).

In some aspects, a cancer that may be prevented, treated, or ameliorated with the compositions contemplated in particular embodiments is a solid tumor. In some embodiments, the cancer is prostate, liver, bile duct, brain, head-and-neck, breast, colon, ovarian, cervical, pancreatic or lung cancer.

Illustrative examples of cancers that may be prevented, treated, or ameliorated with the compositions contemplated in particular embodiments include, but are not limited to, adenomas, carcinomas, sarcomas, leukemias, lymphomas, and multiple myelomas. In some embodiments, the cancer to be prevented, treated, or ameliorated with the compositions contemplated herein include acute lymphocytic leukemia (ALL), acute myeloid leukemia (AML), myeloblastic, promyelocytic, myelomonocytic, monocytic, erythroleukemia, hairy cell leukemia (HCL), chronic lymphocytic leukemia (CLL), and chronic myeloid leukemia (CIVIL), B cell acute lymphocytic leukemia (B-ALL), chronic myelomonocytic leukemia (CMML) and polycythemia vera, Hodgkin lymphoma, nodular lymphocyte-predominant Hodgkin lymphoma and Non-Hodgkin lymphoma, including but not limited to, B-cell non-Hodgkin lymphomas: Burkitt lymphoma, small lymphocytic lymphoma (SLL), diffuse large B-cell lymphoma (DLBCL), follicular lymphoma, immunoblastic large cell lymphoma, precursor B-lymphoblastic lymphoma, and mantle cell lymphoma; and T-cell non-Hodgkin lymphomas: mycosis fungoides, anaplastic large cell lymphoma, Sézary syndrome, and precursor T-lymphoblastic lymphoma; overt multiple myeloma, smoldering multiple myeloma (MGUS), plasma cell leukemia, non-secretory myeloma, IgD myeloma, osteosclerotic myeloma, solitary plasmacytoma of bone, and extramedullary plasmacytoma, renal cell carcinoma (RCC), neuroblastoma, colorectal cancer, breast cancer, ovarian cancer, melanoma, sarcoma, prostate cancer, lung cancer (e.g., non-small cell lung cancer (NSCLC), small cell lung cancer or lung carcinoid tumor), esophageal cancer, hepatocellular carcinoma, pancreatic cancer, astrocytoma, mesothelioma, head and neck cancer, medulloblastoma, and liver cancer.

In some embodiments, the present disclosure provides a method of killing a target cells expressing ASPH comprising genetically modifying a T cell to express an anti-ASPH CAR and administering the anti-ASPH CAR T cell to a subject in need thereof is provided. The infused cell is able to kill disease causing target cells in the recipient. Unlike antibody therapies, CAR T cells are able to replicate in vivo resulting in long-term persistence that can lead to sustained cancer therapy. In the case of T cell-mediated killing, CAR-ligand binding initiates CAR signaling to the T cell, resulting in activation of a variety of T cell signaling pathways that induce the T cell to produce or release proteins capable of inducing target cell apoptosis by various mechanisms. These T cell-mediated mechanisms include (but are not limited to) the transfer of intracellular cytotoxic granules from the T cell into the target cell, T cell secretion of pro-inflammatory cytokines that can induce target cell killing directly (or indirectly via recruitment of other killer effector cells), and up regulation of death receptor ligands (e.g. FasL) on the T cell surface that induce target cell apoptosis following binding to their cognate death receptor (e.g. Fas) on the target cell. In one embodiment, the target cell is a hematopoietic cell, a lymphoid cell, or a myeloid cell. In certain embodiments, the target cell is part of the blood, a lymphoid tissue, or a myeloid tissue. In a particular embodiment, the target cell is a cancer cell that expresses ASPH.

In one embodiment, a method of treating cancer in a subject in need thereof comprises administering an effective amount of a composition comprising genetically modified immune effector cells contemplated herein. The quantity and frequency of administration will be determined by such factors as the condition of the patient, and the type and severity of the patient's disease, although appropriate dosages may be determined by clinical trials.

In one embodiment, the amount of immune effector cells, e.g., T cells, in the composition administered to a subject is at least 0.1×10⁵ cells, at least 0.5×10⁵ cells, at least 1×10⁵ cells, at least 5×10⁵ cells, at least 1×10⁶ cells, at least 0.5×10⁷ cells, at least 1×10⁷ cells, at least 0.5×10⁸ cells, at least 1×10⁸ cells, at least 0.5×10⁹ cells, at least 1×10⁹ cells, at least 2×10⁹ cells, at least 3×10⁹ cells, at least 4×10⁹ cells, at least 5×10⁹ cells, or at least 1×10¹⁰ cells.

In particular embodiments, about 1×10⁹ T cells to about 1×10⁹ T cells, about 2×10⁷ T cells to about 0.9×10⁹ T cells, about 3×10⁷ T cells to about 0.8×10⁹ T cells, about 4×10⁷ T cells to about 0.7×10⁹ T cells, about 5×10⁷ T cells to about 0.6×10⁹ T cells, or about 5×10⁷ T cells to about 0.5×10⁹ T cells are administered to a subject.

In one embodiment, the amount of immune effector cells, e.g., T cells, in the composition administered to a subject is at least 0.1×10⁴ cells/kg of bodyweight, at least 0.5×10⁴ cells/kg of bodyweight, at least 1×10⁴ cells/kg of bodyweight, at least 5×10⁴ cells/kg of bodyweight, at least 1×10⁵ cells/kg of bodyweight, at least 0.5×10⁶ cells/kg of bodyweight, at least 1×10⁶ cells/kg of bodyweight, at least 0.5×10⁷ cells/kg of bodyweight, at least 1×10⁷ cells/kg of bodyweight, at least 0.5×10⁸ cells/kg of bodyweight, at least 1×10⁸ cells/kg of bodyweight, at least 2×10⁸ cells/kg of bodyweight, at least 3×10⁸ cells/kg of bodyweight, at least 4×10⁸ cells/kg of bodyweight, at least 5×10⁸ cells/kg of bodyweight, or at least 1×10⁹ cells/kg of bodyweight.

In particular embodiments, about 1×10⁶ T cells/kg of bodyweight to about 1×10⁸ T cells/kg of bodyweight, about 2×10⁶ T cells/kg of bodyweight to about 0.9×10⁸ T cells/kg of bodyweight, about 3×10⁶ T cells/kg of bodyweight to about 0.8×10⁸ T cells/kg of bodyweight, about 4×10⁶ T cells/kg of bodyweight to about 0.7×10⁸ T cells/kg of bodyweight, about 5×10⁶ T cells/kg of bodyweight to about 0.6×10⁸ T cells/kg of bodyweight, or about 5×10⁶ T cells/kg of bodyweight to about 0.5×10⁸ T cells/kg of bodyweight are administered to a subject.

One of ordinary skill in the art would recognize that multiple administrations of the compositions contemplated herein may be required to effect the desired therapy. For example a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

The administration of the compositions contemplated herein may be carried out in any convenient manner, including by aerosol inhalation, injection, ingestion, transfusion, implantation or transplantation. In some embodiments, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

In one embodiment, a method of treating a subject diagnosed with an ASPH expressing cancer is provided comprising removing immune effector cells from a subject diagnosed with an ASPH expressing cancer, genetically modifying said immune effector cells with a vector comprising a nucleic acid encoding a CAR contemplated herein, thereby producing a population of modified immune effector cells, and administering the population of modified immune effector cells to the same subject. In some embodiments, the immune effector cells comprise T cells.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### EXAMPLES

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### Example 1—Generation of CAR Constructs and Test Expression of CAR's in HT-1080 Cells

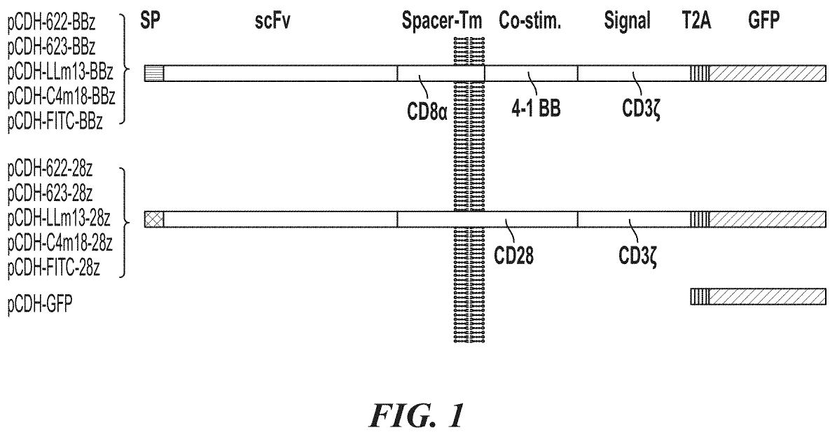

Lentivirus encoding the CAR constructs in FIG. 1 were generated as described below. Synthetic gene fragments encoding codon-optimized versions of the various genes (GeneArt/Invitrogen) were cloned into a lentiviral vector (System Biosciences, SBI). Unique restriction sites enabled easy cloning of the various scFv's into each vector backbone, utilizing either 4-1BB or 28z as co-stimulatory domain. The ASPH-specific scFv's originate from a yeast display library of human antibody-derived scFv sequences selected against ASPH (Wittrup et al.: U.S. Pat. No. 7,413,737; Yeung et al., 2007). Clone 622 and 623 bound to separate epitopes (Yeung et al., 2007). Clones LLm13 and C4m18 were the result of affinity maturation of clone 622 (Wittrup et al.: U.S. Pat. No. 7,413,737). A fluorescein isothiocyanate (FITC) binding scFv was employed as a non-ASPH binding control (Vaughan et al., Nat. Biotechnol. (1996) 14, 309-314).

In order to follow transduction efficiency (and enable facile viral titering) by flow cytometry, the constructs co-expresses GFP as a T2A-GFP fusion, where T2A and furin cleavage sites enables separate CAR and GFP expression from a single open reading frame. The sequences of the CAR's expressed from these constructs are listed in Tables 5 and 6 above, SEQ ID NOs: 40, 42, 44, and 46 (BBz constructs) and SEQ ID NOs: 48-51 (28z constructs). Maxiprep DNA of the lentiviral expression constructs was transfected into the 293TN producer cell line along with accessory packaging plasmids (pPACKH1 Lentivector Packaging Kit; SBI) using PureFection Transfection reagent (SBI) according to the manufacturer's instructions, and supernatant harvested after 2 days. Lentiviral particles were concentrated 100-fold using PEG-It reagent (SBI) before freezing at −80° C.

Transduction of adherent HT-1080 cells were done using TransDux and TransDux MAX Enhancer reagents (SBI) as per the manufacturer's instruction. Briefly, HT-1080 cells were seeded the day before transduction. On the day of transduction (cells around 50-70% confluent) medium was aspired from the cells and replaced with TransDux and TransDux MAX Enhancer combined with culture medium to a final concentration of 1×. Virus diluted in medium was added and plate swirled gently to mix. After overnight incubation at 37° C., fresh media was added and incubated for another 24 h before analysis of CAR expression.

Detection of CAR's on the surface of HT-1080 cells was measured by flow cytometry on a MACSQuant Analyzer 10 instrument (Miltenyi) using biotin-labelled goat anti-human IgG (Fab′)2 (Jackson) and anti-Biotin-APC antibody (Miltenyi). APC and GFP signals were monitored in parallel. GFP expression was measured to ensure the expression levels were not caused by major differences in viral titers. Cells transduced with a non-CAR expressing control construct was used as a negative control for the flow experiments. Viral titers were calculated based on the number of GFP+ cells.

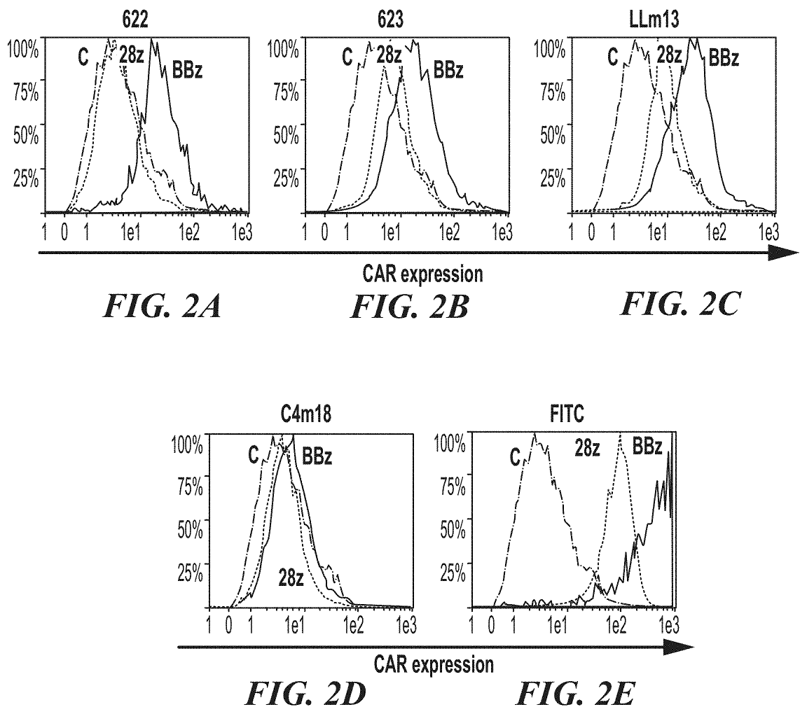

Surprisingly, all 28z series constructs displayed lower CAR expression compared to their BBz counterparts (FIG. 2). Interestingly, C4m18 appeared to express poorly in both contexts (FIG. 2D), while the FITC scFv afforded the best expression levels (FIG. 2E).

### Example 2—Expression of CARs on Human T-Cells

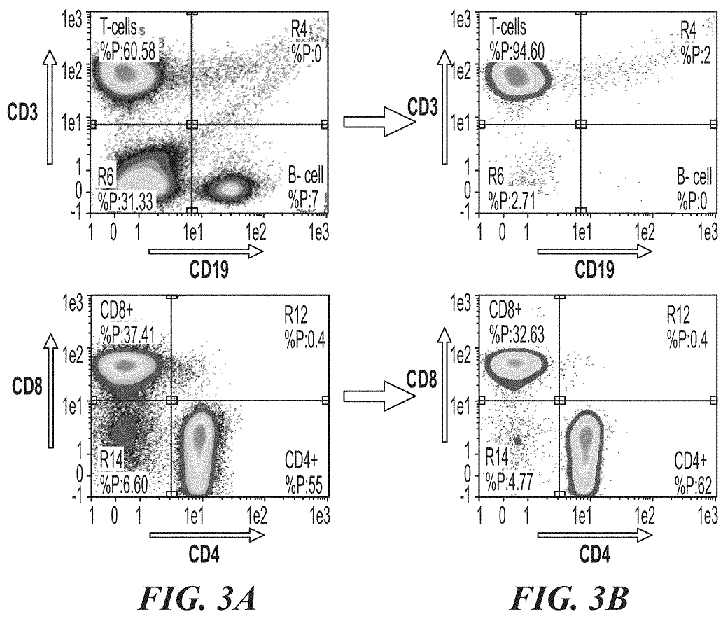

A subset of the lentiviral constructs evaluated in the HT-1080 cell line were transduced into human T-cells. Briefly, CD3+ cells were isolated from PBMC's from human donors using a Pan T Cell Isolation Kit (Miltenyi) as per the manufacturer's instructions. Determination of cell numbers and QC was performed by flow cytometry using the 7-color Immunophenotyping kit (Miltenyi) according to the provided protocol; comparing samples before and after enrichment. FIG. 3 shows the results of a typical T-cell enrichment. Cells before (FIG. 3A) or after (FIG. 3B) isolation were labelled with CD45-VioBlue, CD3-APC, CD19-PE-Vio770, CD4-PerCP, and CD8-APC-Vio770, and analyzed by flow cytometry. Elimination of doublets was done by setting a gate around single cells in forward scatter area (FSC-A) versus forward scatter height (FSC-H). CD45 was used to target all leukocytes. Upper panels show staining for CD3 (pan T-cells) and CD19 (B-cells), and lower panels show the staining of the CD3+ cell subset (by gating) for CD4 (T-helper cells) and CD8 (cytotoxic T cells). In this case, T-cell content was enhanced from 60% to 95% with a largely maintained CD4+/CD8+ ratio.

Purified T cells were grown in TexMACS Medium (Miltenyi) supplemented with 20 IU/mL Human IL-2 IS (Miltenyi). Activation was done at an initial surface density of 1×10⁶ cells/cm² using the T Cell TransAct reagent (nanomatrix conjugated to CD3 and CD28 agonists; Miltenyi) as per the manufacturer's instructions. Cells were incubated with the reagent at 37° C.; 5% CO₂ for 2 days prior to transduction.

Lentiviral transduction was performed using Retronectin-coated plates as per the manufacturers protocol (TaKaRa). After coating overnight, plates were blocked with BSA, viral stock diluted in medium added, and plate centrifuged for 2 hours at 32° C. at 2,000×g to facilitate binding of virus particles to the RetroNectin reagent. After washing in PBS, activated T-cells (2.5×10⁵ cells per well in a 48-well plate; 1 mL per well) were added and plate centrifuged at 32° C./1000×g for 20 min, then moved to 37° C., 5% CO₂ incubator overnight. Cells were split 1:1 in TexMACS medium+IL-2 into a new standard 24w tissue culture plate. Transduced T-cells were split every 2 days into fresh supplemented TexMACS Medium (keeping density around 0.5×10⁶-1×10⁶ cells/cm²).

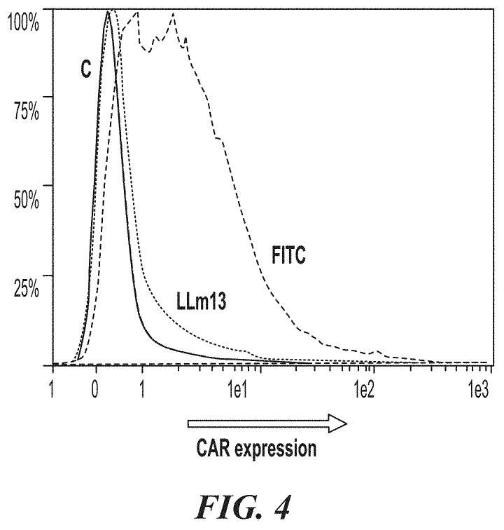

CAR expression was measured 5 days after transduction by flow cytometry as described above. As seen in FIG. 4, CAR expression was much lower on T-cells than observed HT-1080 using a similar number of lentiviral particles. As observed for HT-1080 cells, the FITC scFv containing construct yields significantly higher expression levels compared to the ASPH-specific scFv's on T-cells.

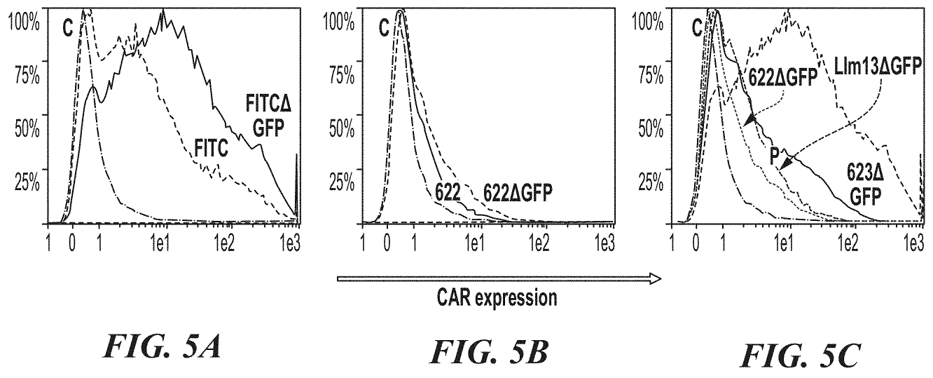

In an attempt to enhance the expression of the CAR's, shorter, less complex expression vectors were generated by deletion of the T2A and GFP sequences. The sequences of the resulting CAR constructs are listed in Table 5, SEQ ID NOs: 41, 43, 45, and 47. Equal amounts of lentivirus encoding the original vs. AGFP constructs were used for transduction of T-cells, and CAR expression monitored as described above. As seen in FIG. 5, these simplified constructs displayed a higher CAR expression level when T-cells were transduced at a similar MOI. FIG. 5A compares the original FITC construct to FITCAGFP; FIG. 5B compares 622 to 622AGFP; uninfected T-cells were stained in parallel as controls (marked with a “C”). The transduced T-cells analyzed in FIG. 5C (622-BBzΔGFP, 623-BBzΔGFP, LLm13-BBzΔGFP and FITC-BBzΔGFP) were tested in a co-culture experiment with an ASPH-expressing cancer cell line.

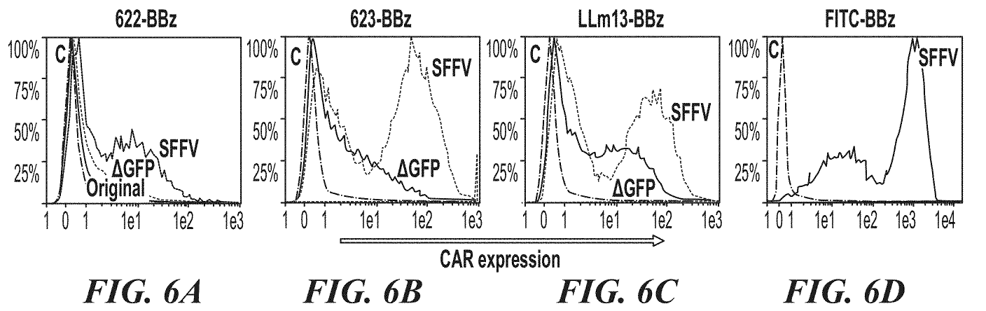

To test the effect of further enhancing CAR expression, we changed the EF1α promoter to the stronger SFFV viral promoter. The comparison of CAR expression from these two promoters in T-cells transduced with similar MOI's were analyzed by flow cytometry as previously described. FIG. 6A affords a direct comparison of the 3 successive generations of the 622-BBz CAR constructs: the original 622-BBz (co-expressing GFP), 622-BBzΔGFP expressed from the EF1α promoter, and the identical 622-BBzΔGFP CAR expressed from the SFFV promoter. The expression level appears to be significantly increased as a result of the increased promoter strength of the SFFV containing construct. Similar observations were made for the 623-BBz, LLm13-BBz, and FITC-BBz CAR constructs (FIG. 6B, FIG. 6C, and FIG. 6D, respectively).

### Example 3—Efficacy of CAR-T Cells in Co-Culture Experiments

The efficacy of the AGFP constructs were tested in a co-culture experiment with the ASPH expressing lung carcinoma cell line H460 (Yeung et al., 2007). 1×10⁴ H460 target cells and 1×10⁵ CAR-T cells (Effector:Target cell ratio=10) were co-cultured in TexMACS medium without IL2 in 100 μL per well in round bottom 96-well culture plates (triplicate samples). After overnight incubation at 37° C., supernatant samples were withdrawn for analysis. Cytotoxicity assay was conducted measuring LDH release from lysed cells using a CytoTox 96 Non-Radioactive Cytotoxicity Assay kit (BioRad). Cytokine release assay was performed with a ProQuantum Human IFN-γ assay kit (Invitrogen).

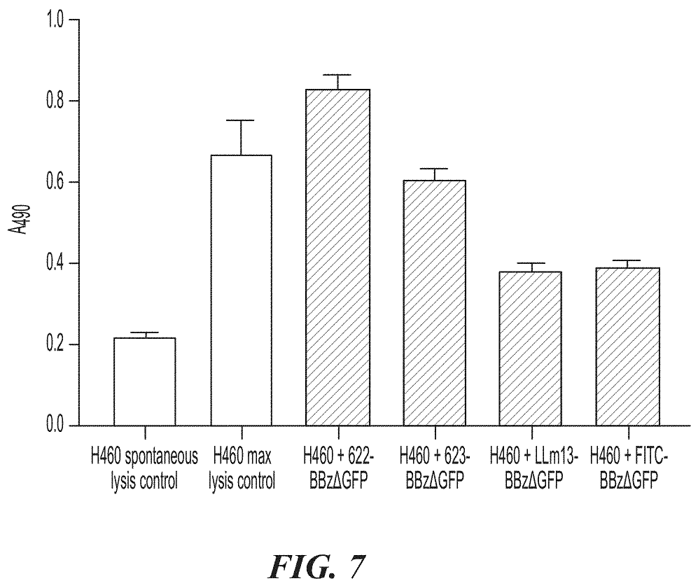

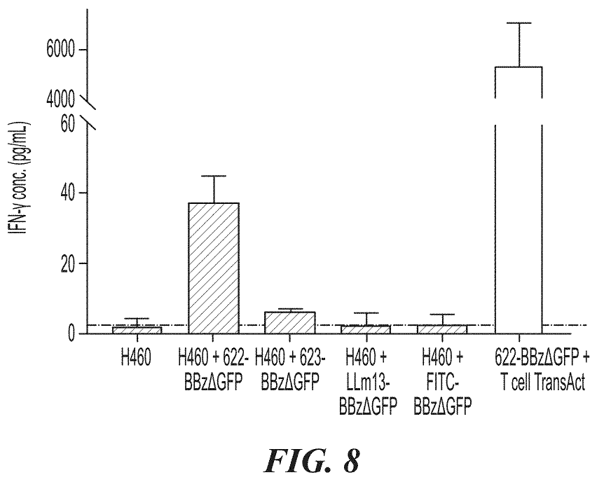

As seen in FIG. 7, increased LDH activity levels over the H460+FITC control CAR-T co-cultures were only seen for H460+622-BBzΔGFP and H460+623-BBzΔGFP co-cultures, with 622-BBzΔGFP showing the most robust lysis level. Surprisingly, even though LLm13 is an affinity matured variant of 622, its level of cell lysis was no different from the co-culture assays with the FITC control construct. Cultures with the same number of H460 cells by themselves were run to determine the level of H460 spontaneous lysis or maximal lysis (by addition of a lysis buffer); FIG. 7, open bars. Consistent with 622-BBzΔGFP yielding the highest level of lysis, this was also the only T-cells showing IFN-γ release over background after co-culturing with H460 (FIG. 8). The level of IFN-γ concentration in the H460+622-BBzΔGFP co-cultures was much lower than in the positive control where the same number of 622-BBzΔGFP CAR-T cell was subjected to robust stimulation with the CD3/CD28 agonist T-cell Trans Act reagent (FIG. 8; open bar).

### Example 4—Efficacy of CAR-T Cells in Additional Co-Culture Experiments

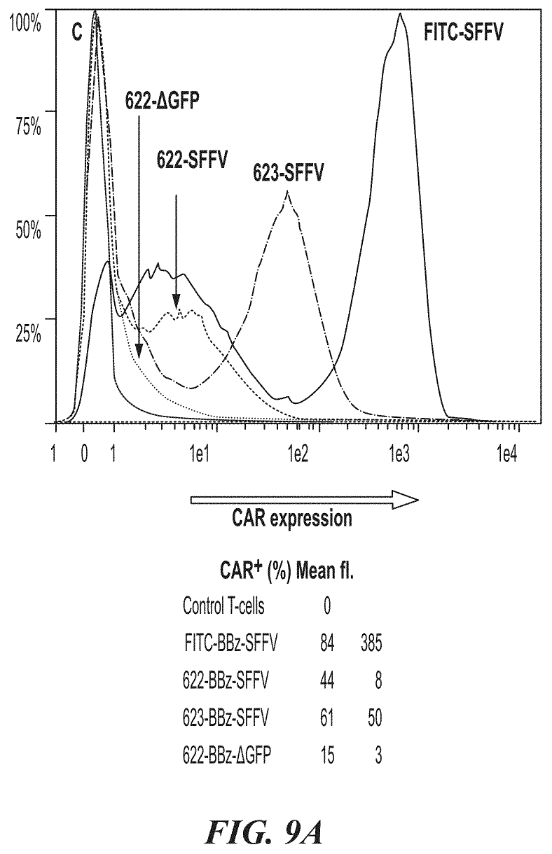

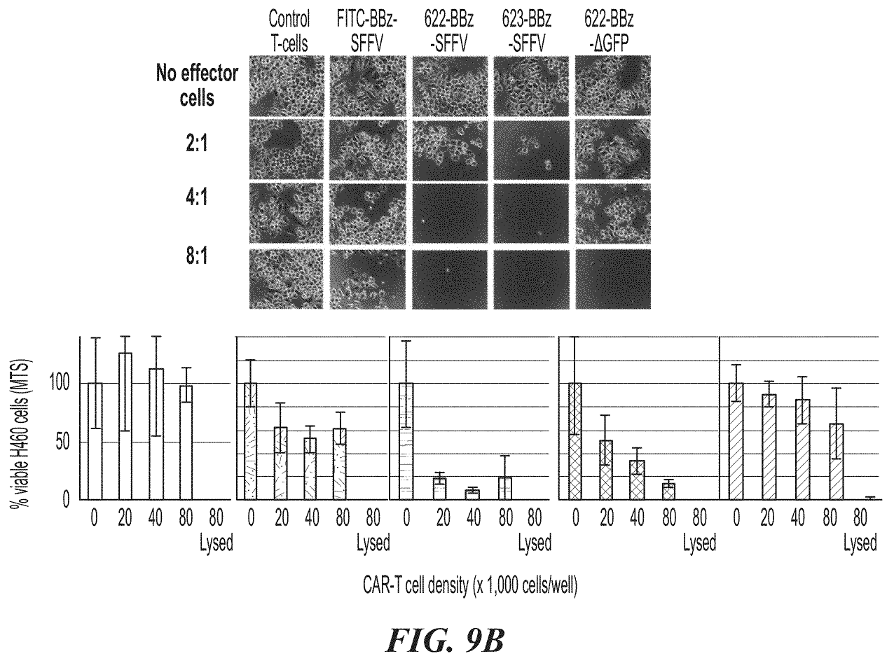

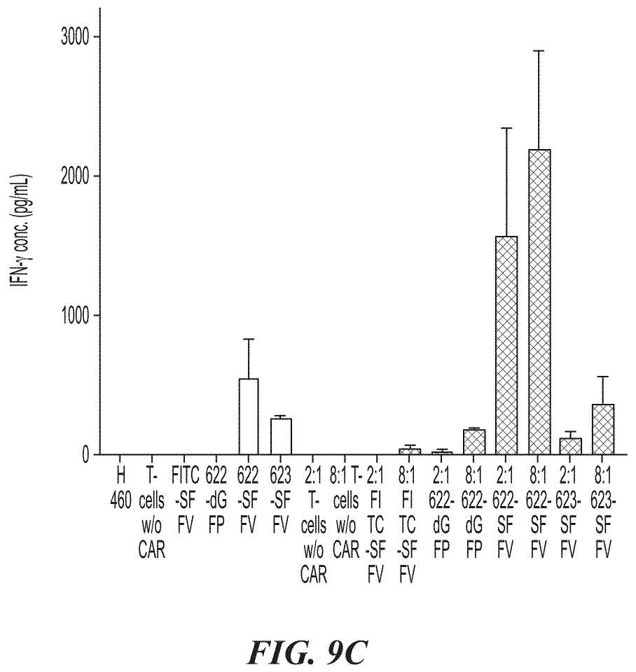

The efficacy of a subset of ASPH-specific CAR constructs were tested in co-cultures with the ASPH expressing lung carcinoma cell line H460. The CAR expression levels on transduced and expanded T-cells were determined as previously described (FIG. 9A). 1×10⁴ H460 target cells were seeded in flat-bottom 96-well culture plates 1 day before the start of the co-culture experiment. CAR-T cells or un-transduced T-cells were added at the various Effector:Target cell ratios stated in the FIG. 9B (triplicate samples). After overnight incubation, culture samples were withdrawn for cytokine release assay (IFN-γ assay as described above), and subsequently T-cells gently washed away. Wells were evaluated by phase-contrast microscopy, followed by measurement of residual live adherent H460 cells using a MTS assay (CellTiter 96 Aqueous One Solution Reagent (Promega)).

Cell killing in a dose-dependent manner was observed for both the 622- and 623-containing constructs (FIG. 9B). The increased CAR expression in the 622-BBz-SFFV constructs compared to the 622-BBz-AGFP construct (FIG. 9A) lead to a higher cell killing activity. Interestingly, despite higher CAR expression, 623-BBz-SFFV appeared to have lower cell killing potency compared to 622-BBz-SFFV (FIG. 9B). Un-transduced control T-cells displayed no cell killing, however a minor effect of the non-binding FITC-BBz-SFFV control construct was observed.

Consistent with 622-BBz-SFFV yielding the highest level of cell killing, this was also the construct displaying the highest level of IFN-γ release in the co-culture experiments (FIG. 9C). IFN-γ release was only slightly higher for 623-BBz-SFFV compared to 622-BBz-AGFP despite much higher CAR expression levels.

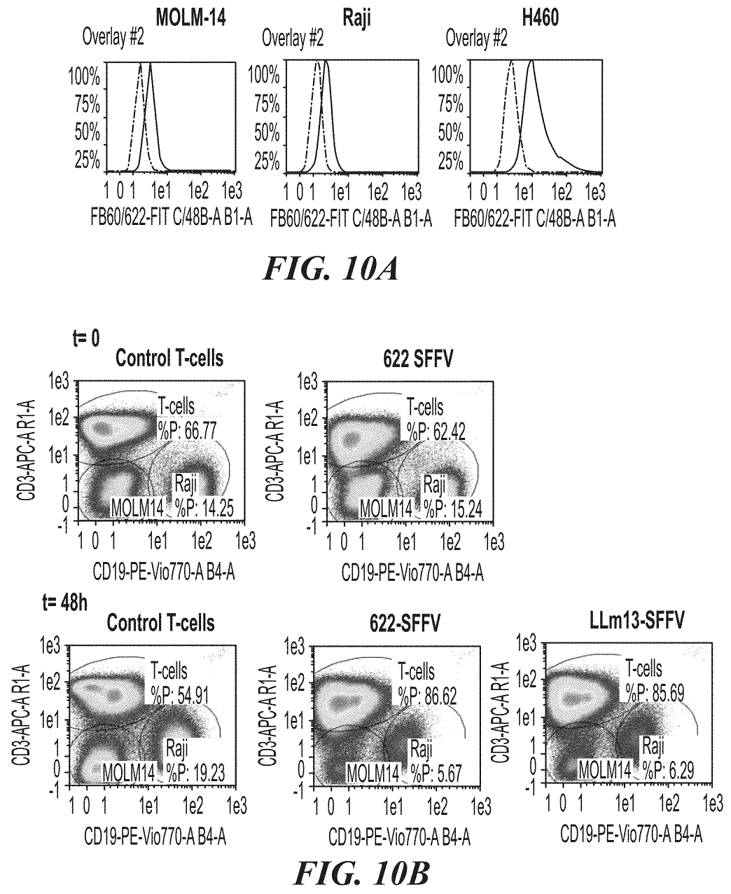

The blood cancer cell lines MOLM-14 (acute myeloid leukemia) and Raji (B-cell line; Burkitt's lymphoma) express ASPH as detected by flow cytometry using FITC-labelled anti-ASPH mAb SNS622 (the 622 scFv reformatted into an IgG1 mAb), although the staining was much lower than for the H460 cell line (stained in parallel for comparison; FIG. 10A). The presence of ASPH on Raji and MOLM-14 indicated these cells could potentially be targeted by ASPH-specific CAR-T cells. Individual populations of T-cells (CD3+), Raji (CD19+) and MOLM-14 (CD3-, CD19-) could be distinguished in the same co-culture by flow cytometry using a subset of the labelled antibodies in the 7-color Immunophenotyping kit as described above. Co-cultures of control (un-transduced) T-cells, 622-BBz-SFFV or LLm13-BBz-SFFV CAR-T cells and MOLM-14+Raji were set up with an effector:total target ratio of 2, and samples from the same co-culture analyzed by flow cytometry immediately (t=0) or after 48 h incubation. FIG. 10B shows the flow analysis and the 3 gates (identical for all 5 data sets) delineating T-cells, MOLM-14 and Raji populations, respectively (the t=0 samples for the LLm13 co-culture were not analyzed). The cell count in each gate is listed in Table 8. As seen, while the MOLM and Raji cell count increases slightly in the control co-culture, these same cells are decreased in the 622-BBz-SFFV and LLm13-BBz-SFFV CAR-T co-cultures, suggesting these ASPH-specific CAR-T's undertake killing of the two target cell lines.

TABLE 8
Cell counts in the 3 gates drawn in FIG. 10B

| | | t = 0 | t = 48 h |
|---|---|---|---|
| Control: Normal T-cells | T-cells | 223148 | 189100 |
| | MOLM-14 | 62734 | 88129 |
| | Raji | 47629 | 66211 |
| | Total count | 333511 | 343440 |
| 622-BBz-SFFV | T-cells | 182389 | 267526 |
| | MOLM-14 | 66734 | 25748 |
| | Raji | 44547 | 17518 |
| | Total count | 293670 | 310792 |
| LLm13-BBz-SFFV | T-cells | | 273007 |
| | MOLM-14 | | 26155 |
| | Raji | | 20030 |
| | Total count | | 319192 |

### Example 5—Specificity of CAR-T Cells in Co-Culture Experiments

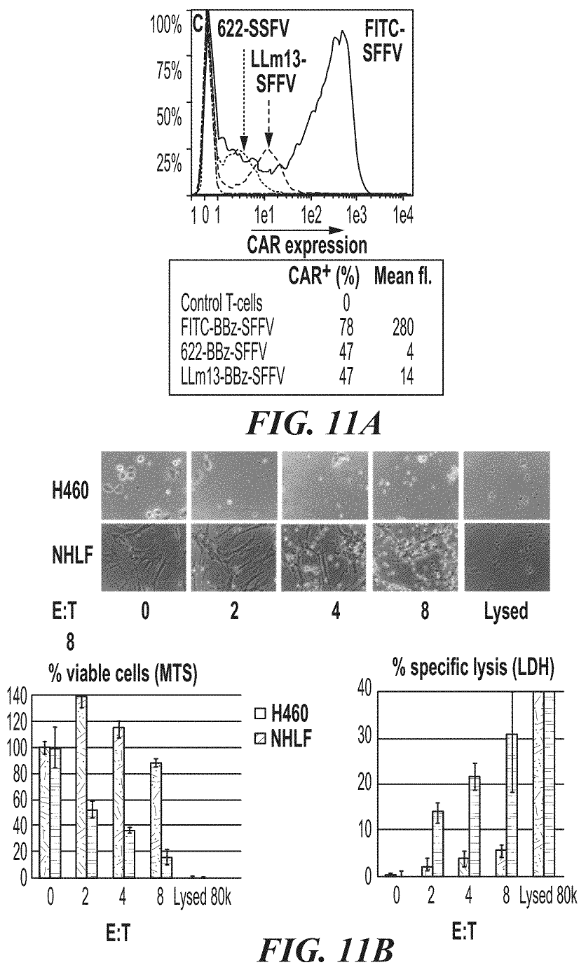

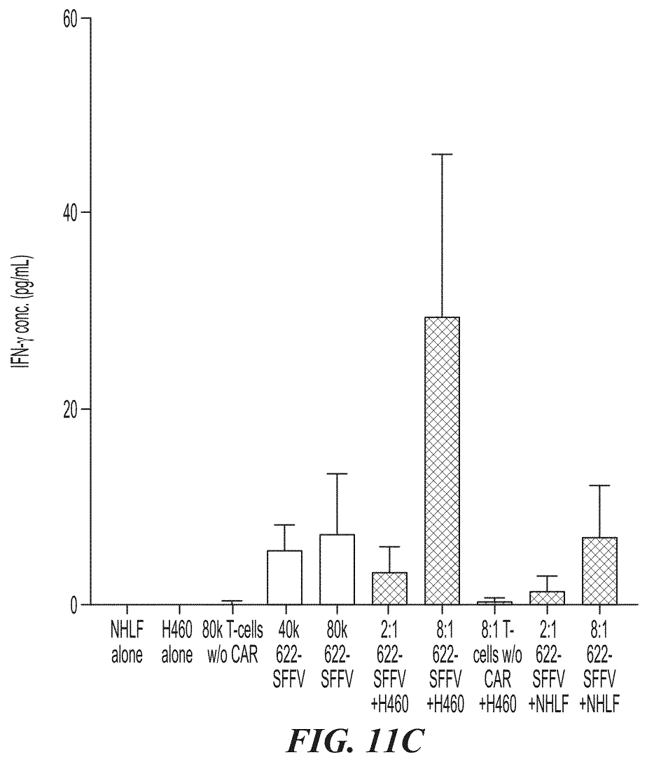

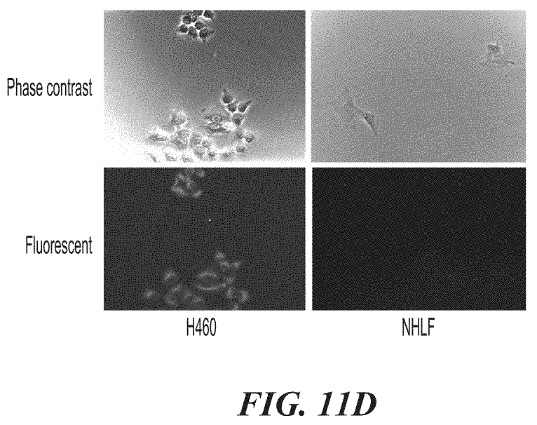

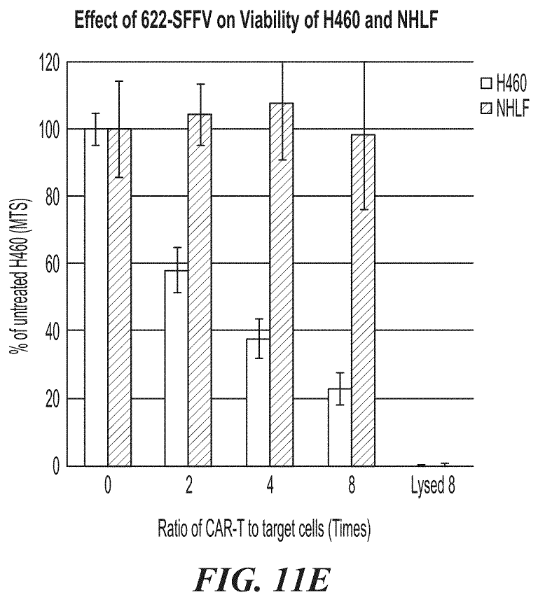

The specificity of 622-BBz-SSFV for ASPH-expressing cells was tested by parallel co-culture experiments with H460 and Normal Human Lung Fibroblasts (NHLF). The CAR expression levels of the utilized CAR-T cell culture were determined as preciously described (FIG. 11A; LLm13-BBz-SFFV and FITC-BBz-SFFV included for comparison). Co-culture experiments were conducted as described, except half (0.5×10⁴) NHLF target cells were seeded compared to H460. As seen in FIG. 11B, while 622-BBz-SFFV offered potent killing of H460 cells as determined both by the MTS and LDH assays, only a marginal effect was observed in the NHLF co-culture assays. Samples from the co-culture assays analyzed for IFN-γ content as described above, indicated a much higher secretion of IFN-γ in the cocultures with H460 compared to NHLF (FIG. 11C). Levels in the 622-BBz-SFFV+NHLF co-cultures were comparable to those seen in the cultures of the same number of 622-BBz-SFFV CAR-T cells by themselves (FIG. 11C). ASPH expression on H460 and NHLF cells was examined with CF488A-conjugated SNS622. Cells were stained with 0.3 μM CF488A-conjugated SNS622 for 1 hour at room temperature. As shown in FIG. 11D, ASPH was undetectable on the surface of NHLF cells under these experimental conditions. In a repeated experiment, the cell killing activity of T-cells expressing the 622-BBz-SFFV CAR on H460 cells and NHLF cells was compared. H460 cells and NHLF cells were treated with 622-BBz-SFFV at different ratios for 24 h day. The cell viabilities were quantitatively evaluated with the MTS assay. Results are shown in FIG. 11E and demonstrate specific cytotoxicity of 622-BBz-SFFV transduced T-cells on ASPH-expressing cells.

1. A chimeric antigen receptor (CAR) comprising:
(a) an extracellular domain comprising an antigen-binding domain that specifically binds to human aspartate β-hydroxylase (ASPH);
(b) a transmembrane region; and
(c) an intracellular domain.

2. The CAR of claim 1, wherein the antigen-binding domain is an antigen-binding fragment of an anti-ASPH antibody.

3. The CAR of claim 2, wherein the antigen-binding fragment comprises a heavy chain variable (VH) region comprising the CDRH1, CDRH2 and CDRH3 and a light chain variable (VL) region comprising the CDRL1, CDRL2 and CDRL3 of an anti-ASPH antibody.

4. The CAR of claim 3, wherein the antigen-binding fragment comprises:
(a) the CDRH1 of SEQ ID NO: 1, the CDRH2 of SEQ ID NO: 2, and the CDRH3 of SEQ ID NO: 3; and the CDRL1 of SEQ ID NO: 4, the CDRL2 of SEQ ID NO: 5, the CDRL3 of SEQ ID NO: 6;
(b) the CDRH1 of SEQ ID NO: 7, the CDRH2 of SEQ ID NO: 2, and the CDRH3 of SEQ ID NO: 8; and the CDRL1 of SEQ ID NO: 4, the CDRL2 of SEQ ID NO: 9, the CDRL3 of SEQ ID NO: 10;
(c) the CDRH1 of SEQ ID NO: 11, the CDRH2 of SEQ ID NO: 12, and the CDRH3 of SEQ ID NO: 13; and the CDRL1 of SEQ ID NO: 4, the CDRL2 of SEQ ID NO: 5, the CDRL3 of SEQ ID NO: 6; or
(d) the CDRH1 of SEQ ID NO: 1, the CDRH2 of SEQ ID NO: 14, and the CDRH3 of SEQ ID NO: 15; and the CDRL1 of SEQ ID NO: 4, the CDRL2 of SEQ ID NO: 5, the CDRL3 of SEQ ID NO: 6.

5. The CAR of claim 3, wherein the antigen-binding fragment comprises:
(a) the VH region comprises the amino acid sequence of SEQ ID NO: 16 and the VL region comprises the amino acid sequence of SEQ ID NO: 17;
(b) the VH region comprises the amino acid sequence of SEQ ID NO: 18 and the VL region comprises the amino acid sequence of SEQ ID NO: 19;
(c) the VH region comprises the amino acid sequence of SEQ ID NO: 20 and the VL region comprises the amino acid sequence of SEQ ID NO: 17; or
(d) the VH region comprises the amino acid sequence of SEQ ID NO: 21 and the VL region comprises the amino acid sequence of SEQ ID NO: 22.

6. The CAR of claim 2, wherein the antigen-binding fragment is a single chain Fv (scFv).

7. The CAR of claim 6, wherein the scFv comprises an amino acid sequence selected from SEQ ID NOs: 23-26.

8. The CAR of claim 1, wherein the antigen-binding domain is a Fibronectin type III domain, a Tn3 protein, a designed ankyrin repeat protein, an affibody, a camelid nanobody, a shark antibody domain, an anticalin, an anti-ASPH aptamer, an EGF-like domain, a human Notch receptor derivative or a human Notch ligand derivative.

9. The CAR of claim 1, wherein the antigen-binding domain cross-competes for binding to ASPH with an anti-ASPH antibody comprising:
(a) a VH region comprising the amino acid sequence of SEQ ID NO: 16 and a VL region comprising the amino acid sequence of SEQ ID NO: 17;
(b) a VH region comprising the amino acid sequence of SEQ ID NO: 18 and a VL region comprising the amino acid sequence of SEQ ID NO: 19;
(c) a VH region comprising the amino acid sequence of SEQ ID NO: 20 and a VL region comprising the amino acid sequence of SEQ ID NO: 17; or
(d) a VH region comprising the amino acid sequence of SEQ ID NO: 21 and a VL region comprising the amino acid sequence of SEQ ID NO: 22.

10. The CAR of claim 1, wherein the extracellular domain further comprises a signal peptide.

11. The CAR of claim 10, wherein the signal peptide comprises a sequence encoding a human CD2, CD3δ, CD3ε, CD3γ, CD3ζ, CD4, CD8α, CD19, CD28, CD37, CD45, 4-1BB, GM-CSFR, IL-2, CD33, Human IgKVIII, Human IgG2 H, Chymotrypsinogen, trypsinogen-2, HSA, Insulin or tPA signal peptide.

12. The CAR of claim 1, wherein the extracellular domain further comprises an additional antigen-binding domain that specifically binds to an antigen other than ASPH.

13. The CAR of claim 12, wherein the additional antigen-binding domain specifically binds to CD19, CD20, CD22, CD5, CD123, CD33, CD70, CD38, CD133, CD138, BCMA (B cell maturation antigen), Mesothelin, GPC3, EpCam, Her2, Muc1, PSCA, CEA, ROR1, GAP, Pan-ErbB, GD2, EphA2, EGFRVIII, IL13Rα2, PSMA, VEGFR2, mucin 16, Lewis-Y or immunoglobulin kappa light chain.

14. The CAR of claim 1, wherein the extracellular domain further comprises a hinge region.

15. The CAR of claim 14, wherein the hinge region comprises a sequence derived from a human CD8α, IgG4, CD28, and/or CD4 sequence.

16. The CAR of claim 1, wherein the transmembrane region comprises a sequence encoding a human CD2, CD3δ, CD3ε, CD3γ, CD3ζ, CD4, CD8α, CD19, CD28, CD37, CD45, 4-1BB or GM-CSFR transmembrane domain.

17. The CAR of claim 1, wherein the intracellular domain comprises a signaling domain.

18. The CAR of claim 17, wherein the signaling domain comprises one or more of an intracellular signaling portion of human CD3 zeta, CD28, CD137, TCR zeta, FcR gamma, FcR beta, CD3 gamma, CD3 delta or CD3 epsilon.

19. The CAR of claim 1, wherein the intracellular domain comprises one, two or three costimulatory domains selected from a human 4-1BB, CD28, CD2, CD27, CD30, CD40, CD40LG (CD40L), DAP-12, OX40, inducible T cell costimulator (ICOS), MyD88, KLRC2 (NKG2C), TNFRS18 (GITR), TNFRSF14 (HVEM), or ITGB2 (LFA-1) costimulatory domain.

20. The CAR of claim 1, wherein the intracellular domain comprises a signaling domain adjacent to a costimulatory domain.

21. A chimeric antigen receptor (CAR) comprising an amino acid sequence selected from SEQ ID NOs: 40-51.

22. A nucleic acid molecule comprising a nucleotide sequence encoding the CAR of claim 1.

23. The nucleic acid molecule of claim 22, further comprising a suicide gene-encoding nucleotide sequence upstream or downstream of the nucleotide sequence encoding the CAR.

24. The nucleic acid molecule of claim 23, wherein the suicide gene-encoding nucleotide sequence encodes an inducible human caspase.

25. The nucleic acid molecule of claim 24, wherein the inducible human caspase is inducible human caspase-9 (iCasp9).

26. The nucleic acid molecule of claim 22, further encoding a cytokine.

27. The nucleic acid molecule of claim 26, wherein the cytokine is IL-15, IL-7, IL-12 or IL-21.

28. The nucleic acid molecule of claim 22, further encoding a costimulatory molecule.

29. The nucleic acid molecule of claim 28, wherein the costimulatory molecule is CD40-L or 4-1BB-L.

30. The nucleic acid molecule of claim 22, further encoding a degrading enzyme.

31. A cell comprising the CAR of claim 1.

32. The cell of claim 31, wherein the cell expresses the CAR on the cell surface.

33. The cell of claim 31, wherein the cell is an immune effector cell.

34. The cell of claim 33, wherein the immune effector cell is a T-cell, a Natural Killer (NK) cell, a Natural Killer (NK)-like cell, a hematopoietic progenitor cell, a peripheral blood (PB) derived T cell or an umbilical cord blood (UCB) derived T-cell.

35. The cell of claim 31, wherein the cell further expresses an inhibitor of an immune checkpoint molecule.

36. The cell of claim 35, wherein the immune checkpoint molecule is PD1, PD-L1, PD-L2, CTLA4, TIM3, CEACAM-1, CEACAM-3, CEACAM-5, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, or TGFR.

37. The cell of claim 31, wherein the cell further expresses a PD-1 dominant negative receptor.

38. The cell of claim 31, wherein the cell further expresses an apoptosis-inducing agent.

39. The cell of claim 38, wherein the apoptosis-inducing agent is a TRAILR2 agonist.

40. A composition comprising the cell of claim 31.

41. A method for expressing a chimeric antigen receptor (CAR) on the surface of a cell, comprising:
(a) obtaining a cell population;
(b) contacting the cell population with a composition comprising a CAR according to claim 1 or a nucleic acid molecule encoding the CAR, under conditions sufficient to transfer the CAR across a cell membrane of at least one cell in the cell population, thereby generating a modified cell population;
(c) culturing the modified cell population under conditions suitable for integration of the CAR; and
(d) expanding and/or selecting at least one cell from the modified cell population that express the CAR on the cell surface.

42. A method for treating cancer in a subject, comprising administering to the subject an effective amount of the cell of claim 31.

43. A method for ameliorating at least one symptom of cancer in a subject, comprising administering to the subject an effective amount of the cell of claim 31.

44. The method of claim 42, wherein the cell is an allogeneic cell.

45. The method of claim 42, wherein the cell is an autologous cell.

46. The method of claim 42, wherein the cancer is ASPH-expressing cancer.

47. The method of claim 42, wherein the cancer is a solid tumor or a hematological malignancy.

48. The method of claim 42, wherein the cancer is prostate, liver, bile duct, brain, head-and-neck, breast, colon, ovarian, cervical, pancreatic or lung cancer.

49. The method of claim 42, further comprising administering a chemotherapeutic agent, radiation and/or an allogeneic stem cell transplant to the subject.

50. The method of claim 49, wherein the cell is engineered to be immune to the chemotherapeutic agent.

51. The method of claim 42, further comprising administering an inhibitor of an immune checkpoint molecule to the subject.

52. The method of claim 51, wherein the immune checkpoint molecule is PD1, PD-L1, PD-L2, CTLA4, TIM3, CEACAM-1, CEACAM-3, CEACAM-5, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, or TGFR.

53. (canceled)
